SEQUENCE LISTING (174 sequences)

SEQ ID NO: 1 - RNA, 22 nt, Homo sapiens
uggaauguaaagaaguauguau

SEQ ID NO: 2 - RNA, 23 nt, Homo sapiens
uggaagacuagugauuuuguugu

SEQ ID NO: 3 - RNA, 22 nt, Homo sapiens
uagcagcacguaaauauuggcg

SEQ ID NO: 4 - RNA, 23 nt, Homo sapiens
caaagugcuuacagugcagguag

SEQ ID NO: 5 - RNA, 23 nt, Homo sapiens
ugugcaaaucuaugcaaaacuga

SEQ ID NO: 6 - RNA, 23 nt, Homo sapiens
ugugcaaauccaugcaaaacuga

SEQ ID NO: 7 - RNA, 23 nt, Homo sapiens
uaaagugcuuauagugcagguag

SEQ ID NO: 8 - RNA, 23 nt, Homo sapiens
caaagugcucauagugcagguag

SEQ ID NO: 9 - RNA, 22 nt, Homo sapiens
uagcuuaucagacugauguuga

SEQ ID NO: 10 - RNA, 22 nt, Homo sapiens
aagcugccaguugaagaacugu

SEQ ID NO: 11 - RNA, 22 nt, Homo sapiens
uucaaguaauccaggauaggcu

SEQ ID NO: 12 - RNA, 21 nt, Homo sapiens
uucaaguaauucaggauaggu

SEQ ID NO: 13 - RNA, 21 nt, Homo sapiens
uucacaguggcuaaguuccgc

SEQ ID NO: 14 - RNA, 21 nt, Homo sapiens
uucacaguggcuaaguucugc

SEQ ID NO: 15 - RNA, 22 nt, Homo sapiens
uagcaccaucugaaaucgguua

SEQ ID NO: 16 - RNA, 22 nt, Homo sapiens
uauugcacauuacuaaguugca

SEQ ID NO: 17 - RNA, 22 nt, Homo sapiens
uauugcacuugucccggccugu

SEQ ID NO: 18 - RNA, 22 nt, Homo sapiens
uauugcacucgucccggccucc

SEQ ID NO: 19 - RNA, 22 nt, Homo sapiens
acugcugagcuagcacuucccg

SEQ ID NO: 20 - RNA, 23 nt, Homo sapiens
caaagugcuguucgugcagguag

SEQ ID NO: 21 - RNA, 22 nt, Homo sapiens
caagcuuguaucuauagguaug

SEQ ID NO: 22 - RNA, 22 nt, Homo sapiens
aacccguagauccgaacuugug

SEQ ID NO: 23 - RNA, 23 nt, Homo sapiens
agcuucuuuacagugcugccuug

SEQ ID NO: 24 - RNA, 21 nt, Homo sapiens
uaaagugcugacagugcagau

SEQ ID NO: 25 - RNA, 22 nt, Homo sapiens
acggguuaggcucuugggagcu

SEQ ID NO: 26 - RNA, 21 nt, Homo sapiens
ucacagugaaccggucucuuu

SEQ ID NO: 27 - RNA, 21 nt, Homo sapiens
cuuuuugcggucugggcuugc

SEQ ID NO: 28 - RNA, 22 nt, Homo sapiens
cagugcaauguuaaaagggcau

SEQ ID NO: 29 - RNA, 22 nt, Homo sapiens
uuugguccccuucaaccagcua

SEQ ID NO: 30 - RNA, 23 nt, Homo sapiens
uauggcuuuuuauuccuauguga

SEQ ID NO: 31 - RNA, 23 nt, Homo sapiens
uauggcuuuucauuccuauguga

SEQ ID NO: 32 - RNA, 21 nt, Homo sapiens
ugagaugaagcacuguagcuc

SEQ ID NO: 33 - RNA, 22 nt, Homo sapiens
ggugcagugcugcaucucuggu

SEQ ID NO: 34 - RNA, 20 nt, Homo sapiens
uacaguauagaugauguacu

SEQ ID NO: 35 - RNA, 22 nt, Homo sapiens
ugagaacugaauuccauggguu

SEQ ID NO: 36 - RNA, 22 nt, Homo sapiens
ugcccuguggacucaguucugg

SEQ ID NO: 37 - RNA, 22 nt, Homo sapiens
ugagaacugaauuccauaggcu

SEQ ID NO: 38 - RNA, 22 nt, Homo sapiens
uugcauagucacaaaagugauc

SEQ ID NO: 39 - RNA, 23 nt, Homo sapiens
aacauucaacgcugucggugagu

SEQ ID NO: 40 - RNA, 23 nt, Homo sapiens
aacauucauugcugucggugggu

SEQ ID NO: 41 - RNA, 22 nt, Homo sapiens
aacauucaaccugucggugagu

SEQ ID NO: 42 - RNA, 23 nt, Homo sapiens
aacauucauuguugucggugggu

SEQ ID NO: 43 - RNA, 22 nt, Homo sapiens
caaagaauucuccuuuugggcu

SEQ ID NO: 44 - RNA, 21 nt, Homo sapiens
cugaccuaugaauugacagcc

SEQ ID NO: 45 - RNA, 23 nt, Homo sapiens
cccaguguucagacuaccuguuc

SEQ ID NO: 46 - RNA, 22 nt, Homo sapiens
uucccuuugucauccuaugccu

SEQ ID NO: 47 - RNA, 22 nt, Homo sapiens
uggaauguaaggaagugugugg

SEQ ID NO: 48 - RNA, 22 nt, Homo sapiens
cugugcgugugacagcggcuga

SEQ ID NO: 49 - RNA, 22 nt, Homo sapiens
acagcaggcacagacaggcagu

SEQ ID NO: 50 - RNA, 21 nt, Homo sapiens
augaccuaugaauugacagac

SEQ ID NO: 51 - RNA, 23 nt, Homo sapiens
agcuacauugucugcuggguuuc

SEQ ID NO: 52 - RNA, 21 nt, Homo sapiens
agcuacaucuggcuacugggu

SEQ ID NO: 53 - RNA, 22 nt, Homo sapiens
acuuuaacauggaagugcuuuc

SEQ ID NO: 54 - RNA, 23 nt, Homo sapiens
uaagugcuuccauguuucagugg

SEQ ID NO: 55 - RNA, 22 nt, Homo sapiens
acuuuaacauggaggcacuugc

SEQ ID NO: 56 - RNA, 22 nt, Homo sapiens
uuuguucguucggcucgcguga

SEQ ID NO: 57 - RNA, 21 nt, Homo sapiens
acuggacuuggagucagaagg

SEQ ID NO: 58 - RNA, 21 nt, Homo sapiens
caaaacgugaggcgcugcuau

SEQ ID NO: 59 - RNA, 22 nt, Homo sapiens
cagcagcaauucauguuuugaa

SEQ ID NO: 60 - RNA, 22 nt, Homo sapiens
aaaccguuaccauuacugaguu

SEQ ID NO: 61 - RNA, 22 nt, Homo sapiens
aacuguuugcagaggaaacuga

SEQ ID NO: 62 - RNA, 22 nt, Homo sapiens
uccuguacugagcugccccgag

SEQ ID NO: 63 - RNA, 20 nt, Homo sapiens
ccauggaucuccaggugggu

SEQ ID NO: 64 - RNA, 22 nt, Homo sapiens
ugaaacauacacgggaaaccuc

SEQ ID NO: 65 - RNA, 23 nt, Homo sapiens
gggguauuguuuccgcugccagg

SEQ ID NO: 66 - RNA, 23 nt, Homo sapiens
uagcagcgggaacaguucugcag

SEQ ID NO: 67 - RNA, 22 nt, Homo sapiens
caaagugccucccuuuagagug

SEQ ID NO: 68 - RNA, 22 nt, Homo sapiens
caaaaaucucaauuacuuuugc

SEQ ID NO: 69 - RNA, 20 nt, Homo sapiens
ucuuacucccucaggcacug

SEQ ID NO: 70 - RNA, 22 nt, Homo sapiens
cacuguuuccuuucugagugga

SEQ ID NO: 71 - RNA, 22 nt, Homo sapiens
ucucacuguagccucgaacccc

SEQ ID NO: 72 - RNA, 21 nt, Homo sapiens
ucucucggcuccucgcggcuc

SEQ ID NO: 73 - RNA, 22 nt, Homo sapiens
cacuguuucaccacuggcucuu

SEQ ID NO: 74 - RNA, 23 nt, Homo sapiens
auacugugaauuucacugucaca

SEQ ID NO: 75 - RNA, 22 nt, Homo sapiens
uccauguuuccuucccccuucu

SEQ ID NO: 76 - RNA, 22 nt, Homo sapiens
ccugcaacuuugccugaucaga

SEQ ID NO: 77 - RNA, 21 nt, Homo sapiens
ucagcuccucucuacccacag

SEQ ID NO: 78 - RNA, 22 nt, Homo sapiens
ucucagcugcugcccucuccag

SEQ ID NO: 79 - RNA, 21 nt, Homo sapiens
caccucuccuggcaucgcccc

SEQ ID NO: 80 - RNA, 20 nt, Homo sapiens
ccacugccuaugccccacag

SEQ ID NO: 81 - DNA, 43 bp, Artificial Sequence
  Description of Artificial Sequence: Synthetic oligonucleotide
ccagtgaatgcgagtccgactaggagagtaggacagcagccgt

SEQ ID NO: 82 - DNA, 65 bp, Artificial Sequence
  Description of Artificial Sequence: Synthetic oligonucleotide
acagttcttcaactggcagcttccagtgaatgcgagtccgactaggagagtaggacagcagccgt

SEQ ID NO: 83 - DNA, 22 bp, Artificial Sequence
  Description of Artificial Sequence: Synthetic oligonucleotide
atacatacttctttacattcca

SEQ ID NO: 84 - DNA, 23 bp, Artificial Sequence
  Description of Artificial Sequence: Synthetic oligonucleotide
acaacaaaatcactagtcttcca

SEQ ID NO: 85 - DNA, 22 bp, Artificial Sequence
  Description of Artificial Sequence: Synthetic oligonucleotide
cgccaatatttacgtgctgcta

SEQ ID NO: 86 - DNA, 23 bp, Artificial Sequence
  Description of Artificial Sequence: Synthetic oligonucleotide
ctacctgcactgtaagcactttg

SEQ ID NO: 87 - DNA, 23 bp, Artificial Sequence
  Description of Artificial Sequence: Synthetic oligonucleotide
tcagttttgcatagatttgcaca

SEQ ID NO: 88 - DNA, 23 bp, Artificial Sequence
  Description of Artificial Sequence: Synthetic oligonucleotide
tcagttttgcatggatttgcaca

SEQ ID NO: 89 - DNA, 23 bp, Artificial Sequence
  Description of Artificial Sequence: Synthetic oligonucleotide
ctacctgcactataagcacttta

SEQ ID NO: 90 - DNA, 23 bp, Artificial Sequence
  Description of Artificial Sequence: Synthetic oligonucleotide
ctacctgcactatgagcactttg

SEQ ID NO: 91 - DNA, 22 bp, Artificial Sequence
  Description of Artificial Sequence: Synthetic oligonucleotide
tcaacatcagtctgataagcta

SEQ ID NO: 92 - DNA, 22 bp, Artificial Sequence
  Description of Artificial Sequence: Synthetic oligonucleotide
acagttcttcaactggcagctt

SEQ ID NO: 93 - DNA, 22 bp, Artificial Sequence
  Description of Artificial Sequence: Synthetic oligonucleotide
agcctatcctggattacttgaa

SEQ ID NO: 94 - DNA, 21 bp, Artificial Sequence
  Description of Artificial Sequence: Synthetic oligonucleotide
acctatcctgaattacttgaa

SEQ ID NO: 95 - DNA, 21 bp, Artificial Sequence
  Description of Artificial Sequence: Synthetic oligonucleotide
gcggaacttagccactgtgaa

SEQ ID NO: 96 - DNA, 21 bp, Artificial Sequence
  Description of Artificial Sequence: Synthetic oligonucleotide
gcagaacttagccactgtgaa

SEQ ID NO: 97 - DNA, 22 bp, Artificial Sequence
  Description of Artificial Sequence: Synthetic oligonucleotide
taaccgatttcagatggtgcta

SEQ ID NO: 98 - DNA, 22 bp, Artificial Sequence
  Description of Artificial Sequence: Synthetic oligonucleotide
tgcaacttagtaatgtgcaata

SEQ ID NO: 99 - DNA, 22 bp, Artificial Sequence
  Description of Artificial Sequence: Synthetic oligonucleotide
acaggccgggacaagtgcaata

SEQ ID NO: 100 - skipped

SEQ ID NO: 101 - DNA, 22 bp, Artificial Sequence
  Description of Artificial Sequence: Synthetic oligonucleotide
ggaggccgggacgagtgcaata

SEQ ID NO: 102 - DNA, 22 bp, Artificial Sequence
  Description of Artificial Sequence: Synthetic oligonucleotide
cgggaagtgctagctcagcagt

SEQ ID NO: 103 - DNA, 23 bp, Artificial Sequence
  Description of Artificial Sequence: Synthetic oligonucleotide
ctacctgcacgaacagcactttg

SEQ ID NO: 104 - DNA, 22 bp, Artificial Sequence
  Description of Artificial Sequence: Synthetic oligonucleotide
catacctatagatacaagcttg

SEQ ID NO: 105 - DNA, 22 bp, Artificial Sequence
  Description of Artificial Sequence: Synthetic oligonucleotide
cacaagttcggatctacgggtt

SEQ ID NO: 106 - DNA, 23 bp, Artificial Sequence
  Description of Artificial Sequence: Synthetic oligonucleotide
caaggcagcactgtaaagaagct

SEQ ID NO: 107 - DNA, 21 bp, Artificial Sequence
  Description of Artificial Sequence: Synthetic oligonucleotide
atctgcactgtcagcacttta

SEQ ID NO: 108 - DNA, 22 bp, Artificial Sequence
  Description of Artificial Sequence: Synthetic oligonucleotide
agctcccaagagcctaacccgt

SEQ ID NO: 109 - DNA, 21 bp, Artificial Sequence
  Description of Artificial Sequence: Synthetic oligonucleotide
aaagagaccggttcactgtga

SEQ ID NO: 110 - DNA, 21 bp, Artificial Sequence
  Description of Artificial Sequence: Synthetic oligonucleotide
gcaagcccagaccgcaaaaag

SEQ ID NO: 111 - DNA, 22 bp, Artificial Sequence
  Description of Artificial Sequence: Synthetic oligonucleotide
atgcccttttaacattgcactg

SEQ ID NO: 112 - DNA, 22 bp, Artificial Sequence
  Description of Artificial Sequence: Synthetic oligonucleotide
tagctggttgaaggggaccaaa

SEQ ID NO: 113 - DNA, 23 bp, Artificial Sequence
  Description of Artificial Sequence: Synthetic oligonucleotide
tcacataggaataaaaagccata

SEQ ID NO: 114 - DNA, 23 bp, Artificial Sequence
  Description of Artificial Sequence: Synthetic oligonucleotide
tcacataggaatgaaaagccata

SEQ ID NO: 115 - DNA, 21 bp, Artificial Sequence
  Description of Artificial Sequence: Synthetic oligonucleotide
gagctacagtgcttcatctca

SEQ ID NO: 116 - DNA, 22 bp, Artificial Sequence
  Description of Artificial Sequence: Synthetic oligonucleotide
accagagatgcagcactgcacc

SEQ ID NO: 117 - DNA, 20 bp, Artificial Sequence
  Description of Artificial Sequence: Synthetic oligonucleotide
agtacatcatctatactgta

SEQ ID NO: 118 - DNA, 22 bp, Artificial Sequence
  Description of Artificial Sequence: Synthetic oligonucleotide
aacccatggaattcagttctca

SEQ ID NO: 119 - DNA, 22 bp, Artificial Sequence
  Description of Artificial Sequence: Synthetic oligonucleotide
ccagaactgagtccacagggca

SEQ ID NO: 120 - DNA, 22 bp, Artificial Sequence
  Description of Artificial Sequence: Synthetic oligonucleotide
agcctatggaattcagttctca

SEQ ID NO: 121 - DNA, 22 bp, Artificial Sequence
  Description of Artificial Sequence: Synthetic oligonucleotide
gatcacttttgtgactatgcaa

SEQ ID NO: 122 - DNA, 23 bp, Artificial Sequence
  Description of Artificial Sequence: Synthetic oligonucleotide
actcaccgacagcgttgaatgtt

SEQ ID NO: 123 - DNA, 23 bp, Artificial Sequence
  Description of Artificial Sequence: Synthetic oligonucleotide
acccaccgacagcaatgaatgtt

SEQ ID NO: 124 - DNA, 22 bp, Artificial Sequence
  Description of Artificial Sequence: Synthetic oligonucleotide
actcaccgacaggttgaatgtt

SEQ ID NO: 125 - DNA, 23 bp, Artificial Sequence
  Description of Artificial Sequence: Synthetic oligonucleotide
acccaccgacaacaatgaatgtt

SEQ ID NO: 126 - DNA, 22 bp, Artificial Sequence
  Description of Artificial Sequence: Synthetic oligonucleotide
agcccaaaaggagaattctttg

SEQ ID NO: 127 - DNA, 21 bp, Artificial Sequence
  Description of Artificial Sequence: Synthetic oligonucleotide
ggctgtcaattcataggtcag

SEQ ID NO: 128 - DNA, 23 bp, Artificial Sequence
  Description of Artificial Sequence: Synthetic oligonucleotide
gaacaggtagtctgaacactggg

SEQ ID NO: 129 - DNA, 22 bp, Artificial Sequence
  Description of Artificial Sequence: Synthetic oligonucleotide
aggcataggatgacaaagggaa

SEQ ID NO: 130 - DNA, 22 bp, Artificial Sequence
  Description of Artificial Sequence: Synthetic oligonucleotide
ccacacacttccttacattcca

SEQ ID NO: 131 - DNA, 22 bp, Artificial Sequence
  Description of Artificial Sequence: Synthetic oligonucleotide
tcagccgctgtcacacgcacag

SEQ ID NO: 132 - DNA, 22 bp, Artificial Sequence
  Description of Artificial Sequence: Synthetic oligonucleotide
actgcctgtctgtgcctgctgt

SEQ ID NO: 133 - DNA, 21 bp, Artificial Sequence
  Description of Artificial Sequence: Synthetic oligonucleotide
gtctgtcaattcataggtcat

SEQ ID NO: 134 - DNA, 23 bp, Artificial Sequence
  Description of Artificial Sequence: Synthetic oligonucleotide
gaaacccagcagacaatgtagct

SEQ ID NO: 135 - DNA, 21 bp, Artificial Sequence
  Description of Artificial Sequence: Synthetic oligonucleotide
acccagtagccagatgtagct

SEQ ID NO: 136 - DNA, 22 bp, Artificial Sequence
  Description of Artificial Sequence: Synthetic oligonucleotide
gaaagcacttccatgttaaagt

SEQ ID NO: 137 - DNA, 23 bp, Artificial Sequence
  Description of Artificial Sequence: Synthetic oligonucleotide
ccactgaaacatggaagcactta

SEQ ID NO: 138 - DNA, 22 bp, Artificial Sequence
  Description of Artificial Sequence: Synthetic oligonucleotide
gcaagtgcctccatgttaaagt

SEQ ID NO: 139 - DNA, 22 bp, Artificial Sequence
  Description of Artificial Sequence: Synthetic oligonucleotide
tcacgcgagccgaacgaacaaa

SEQ ID NO: 140 - DNA, 21 bp, Artificial Sequence
  Description of Artificial Sequence: Synthetic oligonucleotide
ccttctgactccaagtccagt

SEQ ID NO: 141 - DNA, 21 bp, Artificial Sequence
  Description of Artificial Sequence: Synthetic oligonucleotide
atagcagcgcctcacgttttg

SEQ ID NO: 142 - DNA, 22 bp, Artificial Sequence
  Description of Artificial Sequence: Synthetic oligonucleotide
ttcaaaacatgaattgctgctg

SEQ ID NO: 143 - DNA, 22 bp, Artificial Sequence
  Description of Artificial Sequence: Synthetic oligonucleotide
aactcagtaatggtaacggttt

SEQ ID NO: 144 - DNA, 22 bp, Artificial Sequence
  Description of Artificial Sequence: Synthetic oligonucleotide
tcagtttcctctgcaaacagtt

SEQ ID NO: 145 - DNA, 22 bp, Artificial Sequence
  Description of Artificial Sequence: Synthetic oligonucleotide
ctcggggcagctcagtacagga

SEQ ID NO: 146 - DNA, 20 bp, Artificial Sequence
  Description of Artificial Sequence: Synthetic oligonucleotide
acccacctggagatccatgg

SEQ ID NO: 147 - DNA, 22 bp, Artificial Sequence
  Description of Artificial Sequence: Synthetic oligonucleotide
gaggtttcccgtgtatgtttca

SEQ ID NO: 148 - DNA, 23 bp, Artificial Sequence
  Description of Artificial Sequence: Synthetic oligonucleotide
cctggcagcggaaacaatacccc

SEQ ID NO: 149 - DNA, 23 bp, Artificial Sequence
  Description of Artificial Sequence: Synthetic oligonucleotide
ctgcagaactgttcccgctgcta

SEQ ID NO: 150 - DNA, 22 bp, Artificial Sequence
  Description of Artificial Sequence: Synthetic oligonucleotide
cactctaaagggaggcactttg

SEQ ID NO: 151 - DNA, 22 bp, Artificial Sequence
  Description of Artificial Sequence: Synthetic oligonucleotide
gcaaaagtaattgagatttttg

SEQ ID NO: 152 - DNA, 20 bp, Artificial Sequence
  Description of Artificial Sequence: Synthetic oligonucleotide
cagtgcctgagggagtaaga

SEQ ID NO: 153 - DNA, 22 bp, Artificial Sequence
  Description of Artificial Sequence: Synthetic oligonucleotide
tccactcagaaaggaaacagtg

SEQ ID NO: 154 - DNA, 22 bp, Artificial Sequence
  Description of Artificial Sequence: Synthetic oligonucleotide
ggggttcgaggctacagtgaga

SEQ ID NO: 155 - DNA, 21 bp, Artificial Sequence
  Description of Artificial Sequence: Synthetic oligonucleotide
gagccgcgaggagccgagaga

SEQ ID NO: 156 - DNA, 22 bp, Artificial Sequence
  Description of Artificial Sequence: Synthetic oligonucleotide
aagagccagtggtgaaacagtg

SEQ ID NO: 157 - DNA, 23 bp, Artificial Sequence
  Description of Artificial Sequence: Synthetic oligonucleotide
tgtgacagtgaaattcacagtat

SEQ ID NO: 158 - DNA, 22 bp, Artificial Sequence
  Description of Artificial Sequence: Synthetic oligonucleotide
agaagggggaaggaaacatgga

SEQ ID NO: 159 - DNA, 22 bp, Artificial Sequence
  Description of Artificial Sequence: Synthetic oligonucleotide
tctgatcaggcaaagttgcagg

SEQ ID NO: 160 - DNA, 21 bp, Artificial Sequence
  Description of Artificial Sequence: Synthetic oligonucleotide
ctgtgggtagagaggagctga

SEQ ID NO: 161 - DNA, 22 bp, Artificial Sequence
  Description of Artificial Sequence: Synthetic oligonucleotide
ctggagagggcagcagctgaga

SEQ ID NO: 162 - DNA, 21 bp, Artificial Sequence
  Description of Artificial Sequence: Synthetic oligonucleotide
ggggcgatgccaggagaggtg

SEQ ID NO: 163 - DNA, 20 bp, Artificial Sequence
  Description of Artificial Sequence: Synthetic oligonucleotide
ctgtggggcataggcagtgg

SEQ ID NO: 164 - RNA, 22 nt, Homo sapiens
ugagguaguaguuuguacaguu

SEQ ID NO: 165 - RNA, 22 nt, Homo sapiens
agagcuuagcugauuggugaac

SEQ ID NO: 166 - RNA, 22 nt, Homo sapiens
cuuucagucggauguuuacagc

SEQ ID NO: 167 - RNA, 22 nt, Homo sapiens
uguaaacauccuugacuggaag

SEQ ID NO: 168 - RNA, 23 nt, Homo sapiens
agcagcauuguacagggcuauga

SEQ ID NO: 169 - RNA, 21 nt, Homo sapiens
cauaaaguagaaagcacuacu

SEQ ID NO: 170 - RNA, 22 nt, Homo sapiens
ucucccaacccuuguaccagug

SEQ ID NO: 171 - DNA, 65 bp, Artificial Sequence
  Description of Artificial Sequence: Synthetic oligonucleotide
aactcagtaatggtaacggtttccagtgaatgcgagtccgactaggagagtaggacagcagccgt

SEQ ID NO: 172 - DNA, 65 bp, Artificial Sequence
  Description of Artificial Sequence: Synthetic oligonucleotide
caagctgatttacacccggtgaccagtgaatgcgagtccgactaggagagtaggacagcagccgt

SEQ ID NO: 173 - DNA, 19 bp, Artificial Sequence
  Description of Artificial Sequence: Synthetic oligonucleotide
gagagtaggacagcagccg

SEQ ID NO: 174 - DNA, 17 bp, Artificial Sequence
  Description of Artificial Sequence: Synthetic oligonucleotide
ggactcgcattcactgg

(19) United States
(12) Patent Application Publication
(10) Pub. No.: US 2021/0230695 A1
(43) Pub. Date: Jul. 29, 2021

(54) BIOMARKER DETECTION IN PULMONARY HYPERTENSION

(71) Applicant: Mirna Analytics LLC, New York, NY (US)

(72) Inventors: Gabriele GRUNIG, New York, NY (US); Serhiy PYLAWKA, New York, NY (US)

(21) Appl. No.: 17/003,581

(22) Filed: Aug. 26, 2020

Related U.S. Application Data
(63) Continuation of application No. PCT/US2019/020356, filed on Mar. 1, 2019.
(60) Provisional application No. 62/637,659, filed on Mar. 2, 2018.

(51) Int. Cl. C12Q 1/6883

(52) U.S. Cl. CPC C12Q 1/6883; C12Q 2600/118; C12Q 2600/178; C12Q 2600/158

(57) ABSTRACT

The present disclosure relates to methods of identifying miRNAs in patients with pulmonary hypertension, subjecting the patients to therapy, and monitoring disease progression by analyzing miRNAs expression levels. The present disclosure also relates to methods of identifying miRNA profiles in a subject to determine whether the subject is suffering from pulmonary hypertension or is at risk of developing pulmonary hypertension.

### CROSS-REFERENCE

The present application is a continuation of International Application No. PCT/US2019/020356, filed Mar. 1, 2019, which claims the benefit of U.S. Provisional Application No. 62/637,659, filed Mar. 2, 2018, each of which is entirely incorporated herein by reference.

### SEQUENCE LISTING

The instant application contains a Sequence Listing which has been submitted electronically in ASCII format and is hereby incorporated by reference in its entirety. Said ASCII copy, created on Feb. 5, 2021, is named 54003-701_301_updated SL.TXT and is 33,374 bytes in size.

### BACKGROUND

Pulmonary hypertension (PH) is a progressive disease for which there is no cure. Patients diagnosed with PH have a poor prognosis and equally compromised quality of life, with a mean life expectancy of 2 to 5 years from the time of diagnosis if untreated. An increase in pressure in the main pulmonary artery is a common connection between all forms of PH regardless of the underlying cause. PH can occur as a primarily vascular disease or can be associated with conditions that cause pulmonary vascular remodeling and constriction. The World Health Organization (WHO) has classified pulmonary hypertension into five groups based on a combination of patient characteristics, clinical features, and cardiopulmonary hemodynamics. The five WHO groups can be used to inform drug treatment options. However, such drug treatment options are extremely expensive and not sufficiently effective. Efficient and non-invasive methods to determine efficacy of PH treatments are lacking.

### SUMMARY

In some embodiments, the disclosure provides a method comprising: obtaining an expression level of miR-22-3p in a biological sample from a subject, and determining whether the biological sample indicates pulmonary hypertension based on the expression level.

In some embodiments, the disclosure provides a method comprising: a) extracting an miRNA associated with pulmonary hypertension from a biological sample from a subject with pulmonary hypertension; b) amplifying the miRNA; c) determining an expression level of the miRNA; and d) determining an miRNA clustering profile on the expression level of the miRNA.

In some embodiments, the disclosure provides a method comprising: a) obtaining an expression level of an miRNA in a first biological sample from a subject before the subject receives a treatment for pulmonary hypertension, wherein the subject has pulmonary hypertension; b) obtaining an expression level of the miRNA in a second biological sample from the subject after the subject receives the treatment for pulmonary hypertension; and c) observing a difference in the expression level of the miRNA in the first biological sample and the expression level of the miRNA in the second biological sample.

In some embodiments, the disclosure provides a method comprising: a) assaying a biological sample of a subject; b) quantifying an expression level of an miRNA in the biological sample, wherein the miRNA is associated with pulmonary hypertension; and c) determining a risk score of the subject for pulmonary hypertension based at least partially on the expression level.

In some embodiments, the disclosure provides a kit comprising: a) an oligonucleotide that binds an miRNA associated with pulmonary hypertension; b) a reagent for amplifying the miRNA; and c) written instructions for quantifying an expression level of the miRNA and for identifying pulmonary hypertension based on the expression level.

In some embodiments, the disclosure provides a method comprising: a) annealing a circularized probe to an miRNA associated with pulmonary hypertension, wherein the circularized probe comprises a sequence that is at least partially complementary to the miRNA, thereby generating an annealed complex; and b) incubating the annealed complex with a polymerase to generate an amplified rolling circle amplification product.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1A and FIG. 1B illustrate hierarchical clustering of serum miRNA levels determined relative to synthetic spike RNA from samples obtained before (B) or after (A) exercise intervention. FIG. 1A illustrates a full pilot study miRNA panel while FIG. 1B illustrates selected miRNAs. FIG. 1C illustrates hierarchical clustering of serum or plasma miRNA levels determined relative to synthetic spike RNA from samples obtained before (B) or after (A) exercise intervention, or following placebo (P) or oxygen (O) intervention, respectively (n=52 paired samples).

FIG. 2A, FIG. 2B, and FIG. 2C illustrate miRNA levels obtained from serum of patients with several forms of pulmonary arterial hypertension, including idiopathic pulmonary arterial hypertension (iPAH) or with heritable pulmonary arterial hypertension (hPAH), that were taken before and after the patients were subjected to 3 weeks of a therapeutic exercise program.

FIG. 3A and FIG. 3B illustrate the level of miR-22-3p relative to 5S RNA (at 10,000 U) in serum samples of patients with heritable pulmonary arterial hypertension (hPAH) taken before and after the patients were subjected to 3 weeks of supervised exercise training.

FIG. 4A, FIG. 4B, and FIG. 4C illustrate miRNA levels obtained from plasma samples of patients (most of them WHO group I) taken before and after the patients were subjected to 1 week of nightly oxygen or placebo intake therapy.

FIG. 5A and FIG. 5B illustrate the level of miR-22-3p relative to 5S RNA (at 10,000 U) in plasma samples of WHO group I patients, taken before and after the patients were subjected to 1 week of nightly oxygen intake, placebo, or acetazolamide therapy.

FIG. 6 illustrates a heat map analysis of circulating miRNA levels determined from samples of patients subjected to either exercise training or nightly oxygen therapy.

FIG. 7A, FIG. 7B, FIG. 7C, and FIG. 7D illustrate circulating serum miRNA levels obtained from patients subjected to a supervised exercise training intervention study. Data show the levels of miR-22-3p relative to miR-451a (FIG. 7A and FIG. 7B), or the levels of miR-22-3p+miR-21-5p relative to Spike-RNA+miR-451a (FIG. 7C and FIG. 7D).

FIG. 8A, FIG. 8B, FIG. 8C, and FIG. 8D illustrate circulating serum miRNA levels obtained from patients in the oxygen intervention study. FIG. 8A and FIG. 8B show the levels of miR-22-3p relative to miR-451a measured in plasma samples obtained from patients treated with either placebo or oxygen therapy. FIG. 8C and FIG. 8D show the levels of miR-22-3p+miR-21-5p relative to Spike-RNA+miR-451a measured in plasma samples obtained from patients treated with either placebo or oxygen therapy.

FIG. 9 illustrates the directional change of miRNA markers (e.g., miR-22-3p+miR-21-5p relative to Spike-RNA+miR-451a) compared to the distance walked by patients who were subjected to exercise training. The distance walked was measured in meters using a 6-minute walking distance test (6 MWD).

FIG. 10 illustrates the comparison between the directional change of miRNA markers (e.g., miR-22-3p+miR-21-5p relative to Spike-RNA+miR-451a) and the distance walked by patients who were subjected to an oxygen intervention study. The distance walked was measured in meters using a 6-minute walking distance test (6 MWD).

FIG. 11 illustrates a correlation between the change in miRNA markers obtained from biological samples from patients receiving either supervised exercise training or nightly oxygen intake, and the distance walked by the patients during a 6-minute walking distance test. The fold change in miR-22-3p+miR-21-5p relative to Spike-RNA+miR-451a levels were measured for each patient and plotted against the patient's distance walked (measured at baseline in meters).

FIG. 12 illustrates a comparison of miRNA markers obtained from plasma samples of control animals (cattle that did not develop PH in high altitude) and of animals that developed pulmonary hypertension (cattle that developed PH in high altitude).

FIG. 13 illustrates the levels of circulating plasma miR-22-3p relative to miR-451a obtained from plasma samples of cattle in high altitude. Plasma samples were obtained from control cattle (i.e., cattle that are tolerant to high altitude; pulmonary artery pressure (PAP)≤50 mmHg (millimeter of mercury)), from cattle that developed PH (i.e., cattle that are intolerant to high altitude; PAP≥79 mmHg), and from cattle that had intermediate PAP (50 mmHg<PAP<79 mmHg).

FIG. 14 illustrates a schematic representation of rolling circle amplification following specific miRNA sequence driven ligation. The backbone DNA sequence that binds to the miRNA sequence is represented by the thick area containing a break. Once the miRNA binds to the backbone, the ligase converts the linear DNA in a circular DNA.

FIG. 15 illustrates a gel picture of rolling circle amplification reactions.

FIG. 16 illustrates a gel picture of amplicons produced from rolling circle amplification reactions.

FIG. 17 illustrates power calculations performed with G*Power software.

### DETAILED DESCRIPTION

The present disclosure provides a minimally-invasive biomarker tool that can be used to identify subjects with PH and to monitor therapeutic management of PH. This tool provides for earlier and cheaper monitoring capabilities (e.g., monitoring response to drug treatment and physical rehabilitation). The present disclosure can also identify PH in a subject depending on an miRNA level detected from a subject's biological sample. Depending on the identified PH, the subject can then undergo treatment for that specific PH. Improvements of a disease condition can be monitored by measuring an miRNA level, for example, after, or before and after the start of treatment. The miRNA level present in a biological sample from a subject can be compared to an miRNA level present in a biological sample from the same subject taken from a different time point. Alternatively, the miRNA level present in a biological sample from a subject can be compared to an miRNA level taken from a biological sample from another subject with PH, from a healthy subject (e.g., a subject without PH), from a population of subjects (e.g., a population of healthy subjects, a population of subjects with PH, or a population of mixed subjects (e.g., healthy subjects and subjects with PH)), or can be compared to a control.

Disclosed herein are methods for determining a status of pulmonary hypertension in a subject or the presence of pulmonary hypertension in a subject. An exemplery method can include obtaining, assaying, or determining an expression level of at least one miRNA (e.g., miR-22-3p, miR-21-3p, and/or miR-451a) in a biological sample from a subject and determining the status, the presence, or the endotype (molecular or physiological cause) of the pulmonary hypertension of the subject based at least partially on the expression level of the at least one miRNA. The expression level can be determined by, for example, an expression array, next generation sequencing (NGS), quantitative reverse transcriptase polymerase chain reaction (qRT-PCR), northern blot analysis, real-time PCR, in situ hybridization, RNase protection, microarray, or a combination thereof. The method can further include comparing the expression level of the at least one miRNA with the expression level of the at least one miRNA from a different biological sample from the same subject, from a different subject, from a population of subjects, or from a control (e.g., a predetermined value). The different subject can be a healthy subject, a subject with pulmonary hypertension, or a subject without pulmonary hypertension. The population of subjects can be a population of healthy subjects, a population of subjects with pulmonary hypertension, a population of subjects without pulmonary hypertension, or a population of mixed subjects (e.g., subjects with and subjects without pulmonary hypertension). The at least one miRNA can be an miRNA associated with pulmonary hypertension (e.g., an miRNA associated with muscle function, an miRNA associated with blood oxygen content, and/or an miRNA that is associated with or regulates (or is linked to) BMPR2, ALK1, SMAD9, caveolin 1, and/or KCNK3). The method can include obtaining an expression level of miR-22-3p. The method can also include comparing the expression level of miR-22-3p to a reference. The subject can be identified as having PH when the expression level of the at least one miRNA (e.g., miR-22-3p) is reduced or increased as compared to the reference. The reference can be the expression level of the miR-22-3p obtained from a biological sample from a healthy subject, from a subject with pulmonary hypertension, from the same subject taken at a different time point, or from a population of subjects. The expression level can be a plurality of expression levels. The expression level can be of one or more miRNAs. The expression level can be a ratio of one or more miRNAs (e.g., ratio of miR-22-3p/miR-451a, a ratio of (miR-22-3p+miR-21-5p)/miR-451a, or any other ratio of any miRNA of the present disclosure. The method can further include comparing a ratio between the expression level of miR-22-3p to an expression level of at least one other miRNA associated with pulmonary hypertension. The subject can be identified as having PH when the expression level of the ratio is reduced or increased as compared to the reference. The biological sample can be taken before and/or after treatment (e.g., before and/or after exercise training, before and/or after oxygen intake therapy, before and/or after another treatment for pulmonary hypertension, and/or before and/or after another treatment for another disease). The biological sample can be, for example, serum, plasma, or another body fluid.

Disclosed herein are methods for determining pulmonary hypertension or progression of pulmonary hypertension in a subject. An exemplery method can include (a) obtaining a first expression level of an miRNA in a first biological sample from a subject, (b) obtaining a second expression level of the miRNA in a second biological sample from the subject after the subject receives treatment for pulmonary hypertension, and (c) observing a change in expression levels of the miRNA based at least partially on the first expression level and the second expression level. The method can further include determining pulmonary hypertension progression in the subject based at least partially on the first expression level and the second expression level. The method can further include providing treatment to the subject determined to have pulmonary hypertension. The treatment can include exercise training, oxygen intake therapy (e.g., at least about 21% oxygen therapy), a therapeutic treatment, or any combination of treatment. The subject can be a subject with PH, a subject suspected of having PH, or a subject at risk of developing PH. The obtaining in (a) can be performed before the obtaining in (b), or the obtaining in (a) can be performed after commencement of treatment but before the obtaining in (b), or the obtaining in (a) can be performed before commencement of treatment. The biological sample can be, for example, serum, plasma, or another body fluid. The first expression level and the second expression level can be, for example, from at least one miRNA, or from two or more miRNAs. The method can further include calculating a ratio of the expression levels of the miRNAs (e.g., ratio comparing an expression level of one miRNA taken from a biological sample from a subject with pulmonary hypertension to an expression level of the same miRNA taken from another biological sample from the same subject after the subject receives treatment for PH). The ratio can also compare the expression level of two or more miRNAs. The ratio can be compared to a reference, or control. The reference or control can be a predetermined value, or a comparable ratio from the same subject taken at different time points, or from at least one healthy subject, or from at least one subject with pulmonary hypertension, or from at least one subject without pulmonary hypertension, or from a mixture of subjects. The first expression level can include obtaining an expression level of at least one miRNA, at least two miRNAs, at least three miRNAs, or more. The second expression level can include obtaining an expression level of at least one miRNA, at least two miRNAs, at least three miRNAs, or more. The miRNA can be an miRNA associated with pulmonary hypertension (e.g., an miRNA associated with muscle function, an miRNA associated with blood oxygen content, and/or an miRNA that is associated with or regulates (or is linked to) BMPR2, ALK1, SMAD9, caveolin 1, and/or KCNK3). The miRNA associated with pulmonary hypertension can include miR-22-3p, miR-21-5p, and miR-451a.

Disclosed herein are methods for classifying or determining a pulmonary hypertension in a subject. An exemplery method can include (a) obtaining an expression level of an miRNA in a biological sample from a subject with pulmonary hypertension, and (b) classifying the pulmonary hypertension of the subject based at least partially on the expression level. The classification can be based on, for example, the World Health Organization (WHO) pulmonary hypertension classification. For example, pulmonary hypertension can be classified into one or more of five groups. The classification can be based on different PH endotypes, phenotypes, and/or status of PH (e.g., early onset, late onset, presence of PH, or absence of PH). The miRNA can be one or more miRNAs associated with pulmonary hypertension (e.g., an miRNA associated with muscle function, an miRNA associated with blood oxygen content, and/or an miRNA that is associated with or regulates (or is linked to) BMPR2, ALK1, SMAD9, caveolin 1, and/or KCNK3). The miRNA can be indicative of pulmonary hypertension. The method can further include, for example, obtaining a ratio of two or more miRNAs, or a ratio of at least one miRNA from a different biological sample taken from the same subject at different time points (e.g., the expression level of the same miRNA is compared between the biological samples), taken from a different subject, or taken from a population of subjects. The expression level or the ratio can be further compared to a reference. In some cases, comparing to a reference can aid in the classification of the PH.

A method of the present disclosure can be used for obtaining an miRNA expression profile that is indicative of pulmonary hypertension or a status of pulmonary hypertension. An exemplery method can include (a) obtaining a biological sample from a subject having pulmonary hypertension or suspected of having pulmonary hypertension, and (b) detecting an miRNA in the biological sample. Detecting the miRNA in the biological sample can be performed, for example, by capturing the miRNA with a polynucleotide probe. The polynucleotide probe can selectively bind to one or more miRNA(s). The miRNA can be an miRNA associated with pulmonary hypertension. An exemplery method can include (a) extracting a plurality of nucleic acids (e.g., miRNAs) from a biological sample, (b) amplifying the plurality of nucleic acids (e.g., miRNAs), by for example, using an amplification reaction, and (c) determining an expression level of at least one miRNA in the biological sample. The method can also include (d) performing a clustering analysis (e.g., an miRNA clustering profile by heat map) to identify miRNAs that are indicative of pulmonary hypertension or of a status of pulmonary hypertension in the subject. The at least one miRNA can be miR-22-3p, miR-21-5p, and/or miR-451a. The miRNA can be at least one miRNA, at least two miRNAs, at least three miRNAs, at least four miRNAs, at least five miRNAs, at least six miRNAs, at least seven miRNAs, at least eight miRNAs, at least nine miRNAs, at least ten miRNAs, or more. The method can include determining an miRNA clustering profile based at least on the expression level of the at least one miRNA in comparison to a reference. For example, the miRNAs from the biological sample can be compared to a database or to miRNAs that were previously obtained from the same subject, from a different subject, or from a population of subjects. The database can contain miRNA information that was obtained from a healthy subject (e.g., a subject without pulmonary hypertension), a group of healthy subjects, a subject with pulmonary hypertension, a group of subjects with pulmonary hypertension, or a mixture of subjects. The mixture of subjects can be blind or unknown. The biological sample can be taken from a subject, for example, before and/or after treatment (e.g., before and/or after oxygen intake, before and/or after exercise training, before and/or after other treatment for PH, and/or before and/or after treatment for another disease (not PH)).

Another exemplery method of the present disclosure can include (a) purifying a ribonucleic acid (RNA) from a biological sample from a subject with pulmonary hypertension, (b) reverse transcribing the RNA to produce a complementary deoxyribonucleic acid (cDNA), (c) amplifying the cDNA with an miRNA-specific oligonucleotide, and (d) quantifying the cDNA to produce an miRNA expression profile from the biological sample. The expression profile can be an expression profile of pulmonary hypertension. For example, the expression profile can be indicative of an endotype of PH, a phenotype of PH, and/or a status of PH (e.g., early onset, late onset, presence of PH, or absence of PH). The amplifying can be performed with, for example, a polymerase chain reaction (PCR). The miRNA can be one or more miRNAs. The miRNA can be, for example, an miRNA associated with pulmonary hypertension (e.g., an miRNA associated with muscle function, an miRNA associated with blood oxygen content, an miRNA associated with erythrocyte function, an miRNA associated with hypoxia, and/or an miRNA that is associated with or regulates (or is linked to) BMPR2, ALK1, SMAD9, caveolin 1, and/or KCNK3 genes).

Disclosed herein are methods for determining whether a subject has pulmonary hypertension. An exemplery method can include (a) measuring an expression level of an miRNA in a biological sample from a subject, (b) measuring an expression level of a reference (control) miRNA in the biological sample from the subject, (c) calculating a ratio of the expression level of the miRNA to the expression level of the reference (control) miRNA, and (d) comparing the ratio with a corresponding control ratio (or a reference). The corresponding control ratio can be obtained, for example, from a comparable biological sample from the same subject that was taken at a different time point, from a different subject without pulmonary hypertension, from a population of subjects without pulmonary hypertension, from a different subject with pulmonary hypertension, from a population of subjects with pulmonary hypertension, or from a mixture of subjects. The control miRNA can be, for example, an miRNA not associated with pulmonary hypertension or can be an miRNA associated with pulmonary hypertension. The control miRNA or the miRNA can be an miRNA associated with muscle function, an miRNA associated with blood oxygen content, an miRNA associated with erythrocyte function, an miRNA associated with hypoxia, an miRNA that is associated with or regulates (or is linked to) BMPR2, ALK1, SMAD9, caveolin 1, and/or KCNK3 genes. The control miRNA can be an miRNA not associated with muscle function, an miRNA not associated with blood oxygen content, an miRNA not associated with erythrocyte function, an miRNA not associated with hypoxia, an miRNA that does not regulate (or is not linked to) BMPR2, ALK1, SMAD9, caveolin 1, and/or KCNK3 genes. The control miRNA can be, for example, miR-410a and/or Spike RNA.

Disclosed herein are kits that can help with the identification of PH in a subject. An exemplery kit can include (a) an oligonucleotide that binds an miRNA associated with pulmonary hypertension, (b) a reagent for amplifying the miRNA, and (c) written instructions for quantifying an expression level of the miRNA and for identifying pulmonary hypertension based on the expression level. The kit can include various oligonucleotides. The oligonucleotide can specifically bind to at least one miRNA. The miRNA associated with pulmonary hypertension can include miR-22-3p, miR-21-5p, and/or miR-451a. The reagent can include a DNA polymerase. The written instructions can include instructions for comparing miRNAs and can include a predetermined value (e.g., a value for determining whether the expression level of the miRNA is indicative of pulmonary hypertension).

Disclosed herein are methods of treating a subject with pulmonary hypertension. An exemplery method can include (a) measuring an expression level of at least one miRNA in a biological sample from a subject having pulmonary hypertension, or at risk of developing pulmonary hypertension, or suspected of having pulmonary hypertension, (b) detecting the subject as having pulmonary hypertension or at risk of developing pulmonary hypertension when a downregulation or an upregulation is observed in the expression level of the at least one miRNA as compared to a reference, and (c) exposing the subject with pulmonary hypertension or at risk of developing pulmonary hypertension to a pulmonary hypertension treatment. The pulmonary hypertension treatment can be, for example, oxygen intake (e.g., supplemental oxygen comprising at least about 21% oxygen), exercise training, and/or any other treatment for PH. The reference can be a fixed value or the reference can be, for example, selected from the group consisting of (i) the expression level of the at least one miRNA from a subject or a population of subjects with pulmonary hypertension, (ii) the expression level of the at least one miRNA from a healthy subject (e.g., subject without pulmonary hypertension) or a population of healthy subjects, and (iii) the expression level of the at least one miRNA from a subject or a population of subjects with increased blood pressure without a diagnosis of pulmonary hypertension.

Disclosed herein are methods for detecting pulmonary hypertension in a subject. An exemplery method can include (a) assaying a biological sample from a subject, (b) quantifying an expression level of an miRNA in a biological sample from a subject, and (c) determining a risk score of the subject for pulmonary hypertension based at least partially on the expression level of the miRNA. The miRNA can be an miRNA associated with pulmonary hypertension. An miRNA associated with pulmonary hypertension can be an miRNA associated with muscle function, an miRNA associated with blood oxygen content, an miRNA associated with erythrocyte function, an miRNA associated with hypoxia, an miRNA that is associated with or regulates (or is linked to) BMPR2, ALK1, SMAD9, caveolin 1, and/or KCNK3 genes. The miRNA associated with pulmonary hypertension can include miR-22-3p, miR-21-5p, and/or miR-451a. The method can also include comparing the expression level of the miRNA to a reference level, or to the expression level of the same miRNA from a comparable biological sample from a different subject (e.g., a healthy subject or a subject with pulmonary hypertension), or comparing to the expression level of the same miRNA from another biological sample from the same subject (e.g., a comparable biological sample from the same subject but taken at a different time point (e.g., taken before and after exercise training or before and after oxygen treatment)). The biological sample can be, for example, serum, plasma, or another body fluid. The method can additionally include a step of lysing, isolating, or enriching a specific fraction of the biological sample. The miRNA can be one or more miRNAs. In some cases, the expression level of an miRNA is a ratio of one or more miRNAs. The quantifying in (b) can include an expression array, next generation sequencing (NGS), quantitative reverse transcriptase polymerase chain reaction (qRT-PCR), northern blot analysis, real-time PCR, in situ hybridization, RNase protection, microarray, or a combination thereof. The risk score can be determined, for example, based on the expression level of the miRNA and a reference.

Disclosed herein are methods for characterizing a pulmonary hypertension in a subject. An exemplery method can include (a) reverse transcribing an miRNA in a biological sample from a subject, (b) amplifying the miRNA, (c) measuring the level of the miRNA, wherein the miRNA is associated with pulmonary hypertension (e.g., miR-22-3p), and (d) detecting whether the level of the miRNA associated with PH is reduced or increased, as compared to a level of the miRNA associated with PH in a biological sample from a subject or a population of subjects who do not have PH, thereby characterizing the PH in the subject, or determining a risk score of the subject for pulmonary hypertension or a status of PH in the subject if the miRNA associated with pulmonary hypertension is reduced or increased. Disclosed herein are methods of determining the likelihood of PH in a subject or likelihood of a subject to develop PH. An exemplery method can include determining the level of at least one miRNA associated with PH in a subject, wherein an altered level of the at least one miRNA in the subject when compared to a control is indicative of an increased likelihood of PH. The PH can be pulmonary arterial hypertension. The miRNA can be one or more miRNAs associated with PH (e.g., miR-22-3p and miR-21-5p). The status of PH can be early onset, late onset, no detection of PH, or detection/presence of PH. The characterizing can be based on the WHO classification. The risk score can be, for example, indicative of the subject's risk of developing PH.

A method of the present disclosure can be used in combination with an additional screening or detection method. For example, a combination of a biomarker assay (e.g., miRNA detection) of the present disclosure and an additional screening test can provide a higher accuracy, sensitivity, and/or specificity of detection of pulmonary hypertension, compared with that obtained using a screening test alone. An exemplery method can include (a) performing a screening test on a subject to evaluate a risk of developing a health condition by the subject, (b) obtaining a biological sample of the subject, (c) quantifying an expression level of a biomarker in the biological sample of the subject, (d) comparing the expression level of the biomarker to a reference level of the biomarker, (e) combining the result of the screening test and the biomarker comparison, and (f) determining a health state of the subject based on the combined information from the screening test and the biomarker results, or any combination thereof. The additional screening test can include, for example, measuring heart specific proteins (e.g., B-type natriuretic peptide (BNP) or NT-pro-BNP) or can include an imaging test (e.g., using x-rays, sound waves, radioactive particles, pulmonary angiogram, computed tomography, cardiac magnetic resonance, electromagnetic radiation, or magnetic fields) from a tissue, organ, or biological sample of the subject. Heart specific proteins can be detected, for example, in the same sample used for miRNA analysis. Heart specific proteins can be detected by rolling circle amplification. Heart specific proteins can be detected using, for example, aptamer containing probes. Heart specific proteins can be detected at the same time (e.g., in the same reaction) as the detection of miRNAs. Heart specific proteins can be detected using the same sample (or a comparable sample) as the sample used for miRNA detection. The tissue or organ can be, for example, lung or heart. The additional screening test can be, for example, a chest x-ray, echocardiogram, right heart catheterization, or a measurement of the right-heart stress marker (e.g., B-type natriuretic peptide biomarkers). The biological sample can be, for example, serum, plasma, or a body fluid. The health condition can be, for example, pulmonary hypertension. The biomarker can be, for example, an miRNA.

Disclosed herein are methods of determining miRNA levels in a biological sample via rolling circle amplification. An exemplery method can include (a) annealing a padlock probe to an miRNA, wherein the padlock probe comprises one or two terminal regions complementary to at least a portion of the miRNA, thereby generating an annealed-padlock-probe-miRNA (b) incubating the annealed-padlock-probe-miRNA with a ligase under conditions suitable to ligate the ends of the padlock probe, thereby generating a ligated (or circularized) padlock probe annealed to the miRNA, and (c) incubating the ligated (or circularized) padlock probe annealed to the miRNA with a DNA polymerase to generate an amplified rolling circle amplification product. The amplified rolling circle amplification product can be used to detect the miRNA. The padlock probe can be an open or closed circle probe (or a linear probe). The padlock probe can be a closed circle probe (or a linear probe that is circularized after binding to specific miRNA and ligation). The padlock probe can include two terminal regions complementary to the at least a portion of the miRNA. The two terminal sequences of the padlock probe can be designed to hybridize to different sequences within the same miRNA. For example, one terminal sequence of the padlock probe can be designed to hybridize to the 5′ end of the miRNA and the other terminal sequence of the padlock probe can be designed to hybridize to the 3′ end of the miRNA. The incubating of (c) can include adding the DNA polymerase and reagents (e.g., buffer, dNTPs, and BSA) manually or individually. The incubating of (c) can include adding the ligated (or circularized) padlock probe annealed to the miRNA to a freeze-dried cake that contains a DNA polymerase (e.g., phi29 polymerase), nucleotides, salts, buffers, and/or random hexamer primers. In some cases, the incubating of (c) does not include random hexamers. The DNA polymerase can be included in a kit. The DNA polymerase can be included in a freeze-dried cake provided in a kit. For example, a commercially available kit can be used for producing the amplified rolling circle amplification product (e.g., Illustra™ Ready-To-Go™ GenomiPhi™ V3 DNA Amplification Kit from GE Healthcare). The kit can be used to amplify a whole genome from samples that contain small amounts of nucleic acid. The amplified rolling circle amplification product can be a DNA product containing multiple copies of the miRNA sequence. The amplified rolling circle amplification product can be an amplified padlock probe. The amplified rolling circle amplification product can be a mixture of amplified padlock probes. The biological sample can be, for example, serum, plasma, or a body fluid. The biological sample can be purified or not purified. The method can further include sequencing the amplified rolling circle amplification product. The miRNA can be from a biological sample from a subject. The miRNA can be a mixture of miRNAs. The padlock probe can contain, for example, 2 nucleotide modifications (from known or commercially available padlock probes). The padlock probe can be a mixture of padlock probes. The mixture of padlock probes can anneal to the mixture of miRNAs. The mixture of padlock probes can include a mixture of padlock probes, wherein each padlock probe in the mixture is complementary to a specific miRNA. The miRNA can include multiple different miRNAs that can be detected using multiple different padlock probes. Incubating the ligated (or circularized) padlock probe annealed to the miRNA with a DNA polymerase can be performed in the presence of labeled nucleotides. Adding labeled nucleotides can generate a labeled ligated (or circularized) padlock probe, a labeled amplified padlock probe, a labeled miRNA, a labeled amplified rolling circle amplification product, or labeled primers. The amplified rolling circle amplification product can include at least one labeled nucleotide, by for example, adding labeled nucleotides during rolling circle amplification with the DNA polymerase in (c). The labeled nucleotide can be a fluorescently labeled nucleotide. The fluorescence can be detected using by, for example, laboratory instruments or a hand-held fluorescent meter. A variety of labels can be used for labeling nucleic acids and can be used in the detection of rolling circle amplification products. Non-limiting examples of such labels include fluorescent labels, chromogenic labels, radioactive labels, luminescent labels, magnetic labels, and electron-density labels. Labels can be incorporated directly into the amplification product, such as with modified or labeled dNTPs during amplification. Alternatively, the amplification products can be labeled indirectly, such as by hybridization to labeled probes. The rolling circle amplification, or the incubating of (c) can be performed at room temperature (or up to about 30° C.) and from about 1 hour to about 24 hours. The rolling circle amplification, or the incubating of (c) can be performed at room temperature (or about 24° C.) for about 24 hours.

Disclosed herein are methods of determining miRNA levels via rolling circle amplification using a circularized probe. An exemplery method can include (a) annealing a circularized probe to an miRNA, wherein the circularized probe comprises a sequence complementary (or reverse complementary) to at least a portion of the miRNA, thereby generating an annealed-circularized-probe-miRNA (b) incubating the annealed-circularized-probe-miRNA with a DNA polymerase to generate an amplified rolling circle amplification product. The rolling circle amplification, or the incubating of (b) can be performed at room temperature (or up to about 30° C.) for about 2 hours or less. The amplified rolling circle amplification product can be analyzed by qPCR with primers specific to the miRNA. Analysis of a pre-amplicon can be performed using at least one primer specific for the miRNA using qPCR. Another exemplery method can include performing the annealing of (a) and the incubating of (b) in one step (at the same time). For example a method of the present disclosure can include incubating an miRNA, a circularized probe, and a DNA polymerase (concurrently or in one step) to generate an amplified rolling circle amplification product, wherein the circularized probe comprises a sequence complementary (or reverse complementary) to at least a portion of the miRNA. The methods of the present disclosure can include rolling circle amplification. The rolling circle amplification (e.g., rolling circle amplification by performing the annealing and the incubating concurrently) can be performed at room temperature (or up to about 30° C.) for about 2 hours or more. The rolling circle amplification can be performed using labeled nucleotides (e.g., a fluorescently labeled nucleotide). The incubating can include adding the DNA polymerase and reagents (e.g., buffer, dNTPs, and BSA) manually or individually. The incubating can include a freeze-dried cake that contains a DNA polymerase (e.g., phi29 polymerase), nucleotides, salts, buffers, and/or random hexamers (e.g., random hexamer primers). In some cases, the incubating does not include random hexamers. The DNA polymerase can be included in a kit. The DNA polymerase can be included in a freeze-dried cake provided in a kit. For example, a commercially available kit can be used for producing the amplified rolling circle amplification product (e.g., Illustra™ Ready-To-Go™ GenomiPhi™ V3 DNA Amplification Kit from GE Healthcare). The kit can be used to amplify a whole genome from samples that contain small amounts of nucleic acid. The amplified rolling circle amplification product can be a DNA product containing multiple copies of the miRNA sequence. The amplified rolling circle amplification product can be an amplified circularized probe. The amplified rolling circle amplification product can be a mixture of amplified circularized probes. The amplified rolling circle amplification product can be used to detect the miRNA. The miRNA can be from a biological sample. The biological sample can be, for example, serum, plasma, or a body fluid. The biological sample can be purified or not purified. The method can further include sequencing the amplified rolling circle amplification product. The miRNA can be from a biological sample from a subject. The miRNA can be a mixture of miRNAs. The circularized probe can contain, for example, 2 nucleotide modifications (from a known or commercially available probe). The circularized probe can be a mixture of circularized probes. The mixture of circularized probes can anneal to the mixture of miRNAs. The mixture of circularized probes can include a mixture of circularized probes, wherein each circularized probe in the mixture is complementary to a specific miRNA. The miRNA can include multiple different miRNAs that can be detected using multiple different circularized probes. Incubating the miRNA, the circularized probe, and the DNA polymerase can be performed in the presence of labeled nucleotides. Incubating the annealed-circularized-probe-miRNA with the DNA polymerase can be performed in the presence of labeled nucleotides. Adding labeled nucleotides can generate a labeled circularized probe, a labeled amplified circularized probe, a labeled miRNA, a labeled amplified rolling circle amplification product, or labeled primers. The amplified rolling circle amplification product can include at least one labeled nucleotide, by for example, adding labeled nucleotides during rolling circle amplification with the DNA polymerase. The labeled nucleotide can be a fluorescently-labeled nucleotide. The fluorescence can be detected by, for example, using laboratory instruments or a hand-held fluorescent meter. A variety of labels can be used for labeling nucleic acids and can be used in the detection of rolling circle amplification products. Non-limiting examples of such labels include fluorescent labels, chromogenic labels, radioactive labels, luminescent labels, magnetic labels, and electron-density labels. Labels can be incorporated directly into the amplification product, such as with modified or labeled dNTPs during amplification. Alternatively, the amplification products can be labeled indirectly, such as by hybridization to labeled probes. The rolling circle amplification, or the incubating step can be performed at room temperature (or up to about 30° C.) and from about 1 hour to about 24 hours. The rolling circle amplification, or the incubating step can be performed at room temperature (or about 24° C.) for about 24 hours. In some cases, the methods of the present disclosure do not require or include a cDNA conversion step. The circularized probe can include one circularized probe specific for one miRNA or the circularized probe can include a mixture of multiple circularized probes that are specific for multiple miRNAs. The methods disclosed herein can also be used to detect miRNAs and heart specific proteins (e.g., BNP (B-type natriuretic peptide) and NT-pro-BNP (N-terminal pro B-type natriuretic peptide)) from the same sample, by for example, using rolling circle amplification. Heart specific proteins can be detected, for example, in the same sample used for miRNA analysis. Heart specific proteins can be detected by rolling circle amplification. Heart specific proteins can be detected using, for example, aptamer containing probes. Heart specific proteins can be detected at the same time (e.g., in the same reaction) as the detection of miRNAs. Heart specific proteins can be detected using the same sample (or a comparable sample) as the sample used for miRNA detection.

Disclosed herein are methods for reducing the number of false-positive or false-negative results for a health condition. An exemplery method can include (a) obtaining a biological sample from a subject with a positive, negative, or ambiguous result from a screening test that evaluates the subject's risk of developing a health condition, (b) quantifying an expression level of a biomarker (miRNA) in the biological sample of the subject, (c) comparing the expression level of the biomarker to a reference level (e.g., the reference level of the biomarker for the health condition), (d) identifying the result of the screening test as a false-positive or a false-negative for the health condition based on the result from the biomarker comparison. The screening test can include, for example, measuring heart specific proteins (e.g., B-type natriuretic peptide (BNP) or NT-pro-BNP) or an imaging test (e.g., using x-rays, sound waves, radioactive particles, pulmonary angiogram, computed tomography, cardiac magnetic resonance, electromagnetic radiation, or magnetic fields) from a tissue, organ, or body fluid of the subject. The tissue can be, for example, a lung tissue or heart tissue. Heart specific proteins can be detected, for example, in the same sample used for miRNA analysis. Heart specific proteins can be detected by rolling circle amplification. Heart specific proteins can be detected using, for example, aptamer containing probes. Heart specific proteins can be detected at the same time (e.g., in the same reaction) as the detection of miRNAs. Heart specific proteins can be detected using the same sample (or a comparable sample) as the sample used for miRNA detection. The screening test can be, for example, a chest x-ray, echocardiogram, and/or right heart catheterization. The biological sample can be, for example, serum, plasma, or body fluid. The health condition can be, for example, pulmonary hypertension. The biomarker can be, for example, miRNA. The reference level can be obtained by measuring the expression level of the biomarker in one or more subjects with pulmonary hypertension. The reference level can be a predetermined value. The reference level can be determined by a computer. The reference level can be obtained by measuring the expression level of the biomarker in one or more subjects without pulmonary hypertension.

Methods of the present disclosure can provide, for example, a low cost, accurate, non-invasive, and easy to implement test for detection of pulmonary hypertension. Methods of the present disclosure can aid early detection of pulmonary hypertension. Methods of the present disclosure can be useful for subjects with undiagnosed pulmonary hypertension. Methods of the present disclosure can reduce the rate of false positives and false negatives obtained from other pulmonary hypertension diagnosis tests, and can improve the accuracy of pulmonary hypertension diagnosis. In some embodiments, the present disclosure provides a bodily fluid, serum or plasma-based test that includes measuring miRNA from a bodily fluid, a serum or plasma sample of the subject to determine the subject's risk of pulmonary hypertension. In some embodiments, the present disclosure provides a device for performing the methods of the present disclosure. The device can be used to analyze a sample, for example, to generate a biomarker signature of the subject. In some embodiments, the device can be used at a clinic, a hospital, or a testing center.

The present disclosure provides methods of detecting micro-ribonucleic acids (microRNAs or miRNAs) and miRNA profiles in a subject to determine whether the subject has pulmonary hypertension, is suspected of having pulmonary hypertension, or is at risk of developing pulmonary hypertension. Pulmonary hypertension is classified into five groups by the World Health Organization (WHO). Group I is called pulmonary arterial hypertension (PAH), and includes PAH that has no known cause (idiopathic), inherited PAH (i.e., familial PAH (FPAH) or heritable pulmonary hypertension), PAH that is caused by drugs or toxins, and PAH associated with other conditions such as connective tissue diseases (e.g., scleroderma or lupus), HIV infection, liver disease, congenital heart disease, high blood pressure in the liver, or associated with certain infections like schistosomiasis, and sickle cell anemia. Group I can also be caused by rare blood conditions, like pulmonary veno-occlusive disease (PVOD) or pulmonary capillary hemangiomatosis (PCH), and a type of pulmonary hypertension present in babies called persistent pulmonary hypertension of the newborn (PPHN).

Group II pulmonary hypertension is characterized as pulmonary hypertension associated with left heart disease. Long-term problems with the left side of the heart can lead to changes in the pulmonary arteries and cause pulmonary hypertension. Problems with the left side of the heart can include left ventricular systolic dysfunction (when the heart cannot pump blood effectively), left ventricular diastolic dysfunction (when the heart cannot properly relax to allow enough blood to flow in), valvular disease (when the valves of the left side of the heart allow blood to leak), or congenital heart defects (heart defects from birth).

Group III pulmonary hypertension is characterized as PH associated with lung diseases or shortage of oxygen in the body (hypoxia). The common diseases associated with group III pulmonary hypertension are chronic obstructive pulmonary disease (COPD), interstitial lung disease, sleep-disordered breathing (a group of diseases that affect breathing during sleep like obstructive sleep apnea (OSA)), chronic high-altitude exposure, lung developmental abnormalities, and alveolar hypoventilation disorders.

Group IV refers to pulmonary hypertension caused by blood clots obstructing the pulmonary arteries, or chronic thromboembolic pulmonary hypertension (CTEPH). Group IV includes PH due to chronic thrombotic and/or embolic diseases, e.g., PH caused by blood clots in the lungs or blood clotting disorders.

Group V includes PH caused by other disorders or conditions, e.g., blood disorders (e.g., polycythemia vera, essential thrombocythemia), systemic disorders (e.g., sarcoidosis, vasculitis), or metabolic disorders thyroid disease, glycogen storage disease). Pulmonary hypertension can also lead to hypertension in other systems, for example, portopulmonary hypertension, in which patients have both portal and pulmonary hypertension.

Methods of the present disclosure can be used to identify a subject with pulmonary hypertension (PH) including, but not limited to, idiopathic PH, heritable PH, pulmonary arterial hypertension (PAH), systemic sclerosis associated PH, schistosomiasis associated PH, or left heart dysfunction associated PH. In some embodiments, pulmonary hypertension can be associated with or include pulmonary arterial hypertension (PAH). In some cases, PH can be triggered by several risk factors (e.g., several risk factors come together to trigger PH disease). PH can be associated with multiple genes in multiple different molecular pathways that have gene-function altering mutations. Examples include the BMPR2 gene and other genes in the BMPR-transforming growth factor signaling networks, potassium channel dysfunction (KCNK3 gene), transcription factors, and water channel (aquaporin gene). Pulmonary hypertension or pulmonary arterial hypertension can be heritable and/or can include mutations in the BMPR2, ALK1 (or ACVRL1), SMAD9, caveolin 1, and/or KCNK3 genes. Pulmonary arterial hypertension can also be associated with or include idiopathic PAH, familial PAH, collagen vascular disease, congenital systemic-to-pulmonary shunts (including Eisenmenger's syndrome), portal hypertension, HIV infection, drugs and toxins, and/or other diseases (thyroid disorders, glycogen storage disease, Gaucher disease, hereditary hemorrhagic telangiectasia, hemoglobinopathies, myeloproliferative disorders, splenectomy). Pulmonary hypertension can be associated with left heart disease can include left-sided atrial or ventricular heart disease, left-sided valvular heart disease, left ventricular systolic dysfunction, left ventricular diastolic dysfunction, valvular heart disease, congenital/acquired left heart inflow/outflow tract obstruction and congenital cardiomyopathy, and/or congenital/acquired pulmonary venous stenosis. Pulmonary hypertension can be associated with chronic arterial obstruction including chronic thrombotic and/or embolic disease, thromboembolic obstruction of proximal pulmonary arteries, thromboembolic obstruction of distal pulmonary arteries, non-thrombotic pulmonary embolism (tumor, parasites, foreign material), congenital pulmonary artery stenosis, arteritis, angiosarcoma, chronic thromboembolic pulmonary hypertension (CTEPH), and any combination thereof. Pulmonary hypertension can be associated with hematologic diseases (such as chronic hemolytic anemia (including sickle cell disease)), systemic diseases (such as sarcoidosis, pulmonary Langerhans cell histiocytosis, lymphangioleiomyomatosis, neurofibromatosis, vasculitis), metabolic disorders (such as glycogen storage disease, Gaucher disease, and thyroid diseases), pulmonary tumoral thrombotic microangiopathy, fibrosing mediastinitis, chronic kidney failure, or segmental pulmonary hypertension (pulmonary hypertension restricted to one or more lobes of the lungs).

The methods of the present disclosure can also be used to detect or monitor pulmonary hypertension. Pulmonary hypertension can be associated with hypoxia, blood oxygen content, oxygen consumption, high altitude, and/or decreased oxygen tension. In some embodiments, pulmonary hypertension is associated with high altitude. In some embodiments, pulmonary hypertension is associated with oxygen consumption (e.g., decreased oxygen consumption). In some embodiments, pulmonary hypertension is associated with hypoxia.

The methods of the present disclosure can include measuring miRNAs to detect PH, predict the risk of a subject to develop PH, identify molecular mechanisms associated with PH, and/or to characterize PH (endotypes characterized by specific molecular or physiologic pathways that cause PH). The methods of the present disclosure can aid in identifying personalized medicine for PH patients. Determining an miRNA level can also be used to monitor disease progression (e.g., PH) in a subject, or can be used as diagnostics. In some embodiments, the miRNA is a circulating miRNA. MiRNA can be used for early identification of PH, can be used for identifying different PH endotypes, can be used to detect PH or progression of PH before the subject develops physical manifestations related to PH. Detection of an miRNA can involve a non-invasive or a minimally invasive process. The miRNA can be measured before and after treatment. The miRNA can be measured hourly, every two hours, every 12 hours, daily, weekly, twice a week, three times a week, every other week, every three weeks, monthly, every four months, every six months, yearly, or longer. The level of miRNA can change when comparing samples taken before and after treatment (e.g., before and after exercise training or oxygen intake). The change can be an upward change or a downward change. The miRNA can be used as a candidate biomarker. A biomarker can be used to identify pathogenesis that cause diseases, such as PH. A biomarker can also be used to improve disease management or to facilitate personalized therapy. A biomarker can be used in the methods of the present disclosure to monitor PH, to diagnose a subject with having PH, or to characterize PH. For example, classification of PH can be based on different endotypes, phenotype, or the WHO classification system. Classification of PH can also be based on disease status (e.g., early onset of pulmonary hypertension, late onset, no indication of pulmonary hypertension, risk of developing PH, based on endotypes, phenotypes, symptoms, or an miRNA).

A method of the present disclosure can include generating a risk score for a health condition for a subject. For example, a method of the present disclosure can include generating a risk score of a subject for pulmonary hypertension or for developing pulmonary hypertension. The risk score can be indicative of the risk of developing a health condition by the subject. A risk score can be calculated based on results of a biomarker assay. A risk score can be calculated based on the presence or the expression level of at least one miRNA in a biological sample of a subject. A risk score can be calculated based on the comparison of at least one miRNA taken at different time points from the same subject, or taken from different subjects (e.g., a subject with pulmonary hypertension or a healthy subject). A risk score can be calculated based on the ratio of one or more miRNAs or can be calculated based on the level of at least one miRNA compared to a reference value. In some cases, a risk score is determined based on statistical analysis. A risk score can be calculated, combined, and/or adjusted based on data from an additional screening test (e.g., x-ray, echocardiogram, right heart catheterization, and/or measurement of the right-heart stress marker (e.g., B-type natriuretic peptide biomarkers)). A risk score can be provided in conjunction with another test (e.g., 6-minute walking distance test), and the combined information can be used to determine, for example, the probability that a patient has pulmonary hypertension.

A method of the present disclosure can include classifying subjects into one or more groups based on their biomarker signature (e.g., at least one miRNA level) alone or in combination with results from an additional screening test (e.g., x-ray, echocardiogram, right heart catheterization, and/or measurement of the right-heart stress marker (e.g., B-type natriuretic peptide biomarkers)). Classifying subjects into one or more groups can include for example, classifying subjects into different pulmonary hypertension endotypes, or classifying subjects into different disease progression or disease status groups (e.g., early onset of pulmonary hypertension, late onset, no indication of pulmonary hypertension, status based on endotypes, phenotypes, or symptoms). Subjects can be classified into a positive (e.g., subjects having pulmonary hypertension) or a negative (e.g., subjects without pulmonary hypertension) group for a health condition. Subjects can be classified into high risk, low risk, and intermediate risk categories for a health condition. In one example, a biomarker signature (e.g., miRNA expression) can be used to determine that a subject is at a low risk for developing pulmonary hypertension and/or may not need to undergo annual or periodic screening. In another example, a patient can be classified as having a high-risk of developing pulmonary hypertension and/or can be recommended to increase surveillance for pulmonary hypertension detection. In some cases, subjects at high risk of developing pulmonary hypertension can be put into an exercise routine program, can be subjected to oxygen therapy, and/or can start a treatment for pulmonary hypertension.

A method of the present disclosure can provide a risk that can be indicative of a current real-time state of a subject. The real-time state can be related to a given disease state, disease stage, therapy related signature, or physiological condition. Because the risk can be reflective of the current state of the subject, a method of the present disclosure can be performed repeatedly over the subject's life, such as annually, semi-annually, or quarterly. For example, high-risk subjects can be monitored quarterly. A method of the present disclosure can differ from genetic testing, which can be performed once or more in the subject's lifetime. A genetic test (e.g., pulmonary hypertension genetic testing) can be conducted using any cell or biological sample from the subject, and can represent lifetime risk. In some embodiments, a genetic test is not indicative of a subject's current health state, while a method of the present disclosure can determine risk. A method of the present disclosure can be used to detect a change in a subject's condition, for example, a change in improvement or worsening of a condition (e.g., PH in a subject). In some embodiments, the change is not indicative of lifetime risk. In some embodiments, the change is indicative of lifetime risk. In some embodiments, a method of the present disclosure can be used to detect lifetime risk (e.g., risk of a subject to develop PH, risk of PH worsening in a subject, or improvement of PH in a subject).

A method of the present disclosure can have a low false-positive or false-negative rate. In some embodiments, the false-positive or false negative rate for the methods of the present disclosure can be, for example, less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, or less than about 20%.

The sensitivity of a method of the present disclosure can be, for example, about 75%, about 80%, about 83%, about 85%, about 87%, about 90%, about 91%, about 92%, about 93%, about 94%, about 95%, about 96%, about 97%, about 98%, about 99%, about 99.5%, or about 100%. The sensitivity of the methods of the present disclosure can be, for example, at least about 75%, at least about 80%, at least about 83%, at least about 85%, at least about 87%, at least about 90%, at least about 93%, at least about 95%, at least about 96%, at least about 97%, at least about 98%, at least about 99%, or at least about 99.5%. The sensitivity of the methods of the present disclosure can be, for example, greater than about 75%, greater than about 80%, greater than about 83%, greater than about 85%, greater than about 87%, greater than about 90%, greater than about 93%, greater than about 95%, greater than about 96%, greater than about 97%, greater than about 98%, greater than about 99%, or greater than about 99.5%. In some embodiments, the sensitivity of the methods of the present disclosure is about 85%. In some embodiments, the sensitivity of the methods of the present disclosure is greater than about 85%.

The specificity of a method of the present disclosure can be, for example, about 75%, about 80%, about 83%, about 85%, about 87%, about 90%, about 91%, about 92%, about 93%, about 94%, about 95%, about 96%, about 97%, about 98%, about 99%, about 99.5%, or 100%. The specificity of the methods of the present disclosure can be, for example, at least about 75%, at least about 80%, at least about 83%, at least about 85%, at least about 87%, at least about 90%, at least about 93%, at least about 95%, at least about 96%, at least about 97%, at least about 98%, at least about 99%, or at least about 99.5%. The specificity of the methods of the present disclosure can be, for example, greater than about 75%, greater than about 80%, greater than about 83%, greater than about 85%, greater than about 87%, greater than about 90%, greater than about 93%, greater than about 95%, greater than about 96%, greater than about 97%, greater than about 98%, greater than about 99%, or greater than about 99.5%. In some embodiments, the specificity of the methods of the disclosure is about 95%. In some embodiments, the specificity of the methods of the disclosure is greater than about 95%.

The accuracy of a method of the present disclosure can be, for example, about 75%, about 80%, about 83%, about 85%, about 87%, about 90%, about 91%, about 92%, about 93%, about 94%, about 95%, about 96%, about 97%, about 98%, about 99%, about 99.5%, or 100%. The accuracy of the methods of the present disclosure can be, for example, at least about 75%, at least about 80%, at least about 83%, at least about 85%, at least about 87%, at least about 90%, at least about 93%, at least about 95%, at least about 96%, at least about 97%, at least about 98%, at least about 99%, or at least about 99.5%. The accuracy of methods of the disclosure can be, for example, greater than about 75%, greater than about 80%, greater than about 83%, greater than about 85%, greater than about 87%, greater than about 90%, greater than about 93%, greater than about 95%, greater than about 96%, greater than about 97%, greater than about 98%, greater than about 99%, or greater than about 99.5%. In some embodiments, the accuracy of the methods of the present disclosure is about 90%. In some embodiments, the accuracy of the methods of the disclosure is greater than about 90%.

The methods of the present disclosure can include determining an miRNA level in a sample from a subject (e.g., a biological sample). A subject can be a subject who is clinically diagnosed with PH, can include a naïve subject (e.g., a subject not previously treated for PH, particularly one who has not previously received exercise treatment or oxygen therapy), and a subject who has failed prior treatment for PH. The biological sample can be from a subject with pulmonary hypertension or at risk of developing pulmonary hypertension. The biological sample can be from a healthy subject or from a subject not suspected of having pulmonary hypertension. The biological sample can be predetermined or can be from a reference (e.g., from a sample bank, or from a healthy subject, from a subject without PH, or from another subject with pulmonary hypertension).

A biological sample can be from blood, serum, plasma, urine, sweat, tears, saliva, sputum, or any combination thereof. In some embodiments, a biological sample can be saliva. In some embodiments, a biological sample can be blood. Non-limiting examples of a biological sample include saliva, whole blood, peripheral blood, plasma, serum, ascites, cerebrospinal fluid, sweat, urine, tears, buccal sample, cavity rinse, sputum, organ rinse, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, or other lavage fluids. A biological sample can also include the blastocyl cavity, umbilical cord blood, or maternal circulation which can be of fetal or maternal origin. The biological sample can also be a tissue sample or biopsy. In some cases, the sample can be blood, whole blood, a fraction of blood, plasma, serum, or any combination thereof. In some cases, an miRNA can be measured from a biological sample, a plasma sample, a serum sample, a whole blood sample, from a fraction of blood sample, or any combination thereof. In some cases, biological samples can include, but are not limited to extracellular vesicles, cultured cells, formalin-fixed paraffin-embedded (FFPE) tissue, plants, yeast, bacteria, and viral samples. In some cases, biological samples can be a liquid or a cell-free sample. In some cases, an miRNA can be measured from different biological samples, from comparable biological samples from different subjects, or from the same biological sample collected at different time points. In some cases, an RNA sample can also come from non-biological sources such as synthetic reactions.

Collection of a biological sample can be performed in any suitable setting, for example, hospitals, home, clinics, pharmacies, and diagnostic labs. A biological sample can be transported by mail or courier to a central clinic for analysis. A biological sample can be stored under suitable conditions prior to analysis.

An miRNA can be used to predict clinical improvements of a subject suffering from PH. For example, treatment can have an effect on a specific miRNA level in a subject. Treatment can include, but is not limited to, exercise, oxygen intake, nightly oxygen intake, daily oxygen intake, drugs for treating pulmonary hypertension, combination therapy, or any combination thereof. Treatment can also include the use of a breathing stabilizer, such as an acetazolamide. The miRNA level can overlap with another predictor of improvement of pulmonary hypertension following therapy. MiRNAs can also be determined from a sample taken from a subject treated with a breathing stabilizer (e.g., an acetazolamide) or a drug used for treating pulmonary hypertension. In some embodiments, the miRNA is determined from a subject who underwent exercise training. In some embodiments, the miRNA is determined from a subject who was subjected to oxygen intake therapy (e.g., nightly oxygen intake). In some embodiments, the miRNA is measured from a sample obtained before treatment, after treatment, or both (e.g., before and after treatment with a breathing stabilizer, oxygen intake, exercise training, drug treatment, and/or combination therapy). In some embodiments, an miRNA is upregulated or downregulated after treatment (e.g., after a subject with PH is subjected to exercise training, or oxygen intake). In some embodiments, the miRNA that is upregulated or downregulated after treatment is an miRNA associated with pulmonary hypertension (e.g., an miRNA associated with muscle function, an miRNA associated with blood oxygen content, an miRNA associated with erythrocyte function, an miRNA associated with hypoxia). In some embodiments, the miRNA that is upregulated or downregulated after treatment is an miRNA associated with the regulation or expression of bone morphogenetic protein receptor 2 (BMPR2), ALK1 (or ACVRL1), SMAD9, caveolin 1, and/or KCNK3. For example, an miRNA that can be downregulated (or upregulated) after treatment can include, but is not limited to, hsa-miR-100-5p, hsa-miR-93-5p, hsa-miR-92b-3p, hsa-miR-20a-5p, hsa-miR-17-5p, hsa-miR-130a-3p, hsa-miR-27a-3p, hsa-miR-106b-5p, hsa-miR-19a-3p, hsa-miR-7-5p, hsa-miR-20b-5p, hsa-miR-19b-3p, hsa-miR-32-5p, hsa-miR-192-5p, and hsa-miR-215-5p.

The miRNA can be an miRNA associated with the regulation or expression of bone morphogenetic protein receptor 2 (BMPR2), ALK1 (or ACVRL1), SMAD9, caveolin 1, and/or KCNK3. For example, an miRNA associated with the regulation or expression of BMPR2 can include, but is not limited to, miR-20 (e.g., miR-20a-5p), miR-21-5p, miR-129-5p, miR-92a-3p, miR-19b-3p, miR-19a-3p, miR-17-5p, miR-100-5p, miR-302c-3p, miR-130a-3p, miR-181c-5p, miR-128-3p, miR-215-5p, miR-7-5p, miR-192-5p, miR-93-5p, miR-26b-5p, miR-93-3p, miR-135a-5p, miR-181a-5p, miR-106b-5p, miR-20b-5p, miR-519d-3p, miR-32-5p, miR-181b-5p, miR-181d-5p, miR-92b-3p, miR-27a-3p, miR-27b-3p, miR-548c-3p, miR-135b-5p, miR-4716-5p, miR-4772-3p, miR-3615, miR-1304-3p, miR-125b-1-3p, miR-6890-3p, miR-6800-3p, miR-6787-3p, miR-6736-3p, miR-4693-5p, miR-103a-2-5p, miR-302d-5p, miR-302b-5p, miR-550b-3p, miR-892c-3p, miR-4676-3p, miR-452-5p, miR-100-3p, miR-490-5p, miR-153-3p, miR-204, miR-135a, miR-375 (e.g., miR-375-3p), miR-494 (e.g., miR-494-3p), miR-17/92 cluster, and miR-302/367 cluster.

An miRNA associated with the regulation or expression of caveolin 1 can include, for example, but is not limited to, miR-34c-5p, miR-34b-5p, miR-124-3p, miR-103a-3p, miR-7-5p, miR-26b-5p, miR-199a-5p, miR-199a-3p, miR-203a-3p, miR-107, ssc-miR-199a-5p, miR-192-5p, miR-17-5p, miR-20 (e.g., miR-20a-5p), miR-93-5p, miR-106a-5p, miR-194-5p, miR-106b-5p, miR-20b-5p, miR-526b-3p, miR-519d-3p, miR-3609, miR-548ah-5p, miR-4796-3p, miR-3973, miR-873-5p, miR-520h, miR-520g-3p, miR-4463, miR-1238-3p, miR-6749-3p, miR-6792-3p, miR-4691-5p, miR-627-3p, miR-660-3p, miR-5193, miR-670-3p, miR-4277, miR-584-3p, miR-5004-3p, miR-1261, miR-4791, miR-3201, miR-766-5p, miR-3140-3p, miR-4722-5p, miR-4468, miR-4673, miR-4645-5p, miR-4692, miR-4514, miR-4459, miR-556-5p, miR-208b-5p, miR-208a-5p, miR-6165, miR-6753-5p, miR-1911-3p, miR-338-5p, miR-4517, and mmu-miR-124-3p.

An miRNA associated with the regulation or expression of SMAD9 can include, for example, but is not limited to, miR-106b-5p, miR-203a-3p, miR-574-5p, miR-653-5p, miR-5585-3p, miR-190a-3p, miR-6867-5p, miR-223-5p, miR-511-3p, miR-5011-5p, miR-1277-5p, miR-665, miR-887-5p, miR-6780a-5p, miR-6779-5p, miR-3689c, miR-3689b-3p, miR-3689a-3p, miR-30b-3p, miR-1273h-5p, miR-6788-5p, miR-30c-2-3p, miR-30c-1-3p, miR-6799-5p, miR-6883-5p, miR-6785-5p, miR-4728-5p, miR-149-3p, miR-7106-5p, miR-7160-5p, miR-4722-5p, miR-6884-5p, miR-485-5p, miR-1827, miR-4649-3p, miR-4768-3p, miR-4478, miR-4419b, miR-3929, miR-940, miR-6893-5p, miR-6808-5p, miR-890, miR-34b-3p, and miR-606.

An miRNA associated with the regulation or expression of KCNK3 can include, for example, but is not limited to, miR-6788-5p, miR-30c-2-3p, miR-30c-1-3p, miR-6778-5p, miR-1233-5p, miR-6766-5p, miR-6756-5p, miR-608, miR-4651, miR-7110-5p, miR-6842-5p, miR-6752-5p, miR-6825-5p, miR-6785-5p, miR-6883-5p, miR-4728-5p, miR-8085, miR-149-3p, miR-6731-5p, miR-6878-5p, miR-4763-3p, miR-1207-5p, miR-6722-3p, miR-1909-3p, miR-4707-5p, miR-6732-5p, miR-4296, miR-4322, miR-4265, miR-4417, miR-6816-5p, miR-3196, miR-3180-3p, miR-3180, miR-3656, miR-3621, miR-423-5p, miR-3184-5p, miR-365b-5p, miR-365a-5p, miR-8052, miR-3199, miR-6778-3p, miR-150-5p, miR-6814-5p, and miR-3691-3p.

An miRNA associated with the regulation or expression of ACVRL1 can include, for example, but is not limited to, miR-6833-3p, miR-4768-5p, miR-6773-5p, miR-6724-5p, miR-6873-3p, miR-4684-5p, miR-296-5p, miR-942-5p, miR-6817-3p, miR-7110-3p, miR-5088-3p, miR-6756-3p, miR-3127-3p, miR-1237-5p, miR-128-1-5p, miR-128-2-5p, miR-4488, miR-4505, miR-4514, miR-4690-5p, miR-4692, miR-4697-5p, miR-4731-5p, miR-5787, miR-637, miR-6808-5p, miR-6846-5p, miR-6848-5p, miR-6877-5p, miR-6893-5p, miR-940, miR-1224-5p, miR-4751, miR-4753-5p, miR-5004-5p, and miR-7160-5p.

The miRNA can be an miRNA associated with blood oxygen content (e.g., an miRNA associated with erythrocyte function, or hypoxia, or associated with oxygen tension, oxygen consumption, or oxygen utilization). The miRNA can be an miRNA associated with erythrocyte function, muscle function, and/or hypoxia. The methods of the present disclosure can include measuring at least one miRNA associated with blood oxygen content (e.g., an miRNA associated with erythrocyte function or hypoxia), at least one miRNA associated with muscle function, at least one miRNA associated with the regulation or expression of BMPR2, ALK1 (or ACVRL1), SMAD9, caveolin 1, KCNK3, and any combination thereof. The miRNA can also be a reference miRNA. A reference miRNA can include a predetermined miRNA, a standard miRNA, an miRNA not associated with PH, an miRNA associated with PH, or an miRNA provided in a kit. Non-limiting examples of miRNA associated with erythrocyte function include miR-16 (e.g., miR-16-5p), miR-92, miR-144 (e.g., miR-144-3p), miR-221 (e.g., miR-221-3p), miR-222 (e.g., miR-222-3p), miR-451, and miR-486. MiR-92 can be miR-92a. MiR-451 can be miR-451a. MiR-486 can be miR-486-5p. Examples of miRNAs associated with muscle function include, but are not limited to, miR-1 (e.g., miR-1-3p), miR-21 (e.g., miR-21-5p), miR-22 (e.g., miR-22-3p), miR-26a (e.g., miR-26a-5p), miR-29a (e.g., miR-29a-3p), miR-133, miR-143, miR-143-5p, miR-143-5p, miR-146, miR-181, miR-204, miR-206, miR-214 (e.g., miR-214-3p), miR-222 (e.g., miR-222-3p), miR-378 (e.g., miR-378a-3p), miR-424, miR-424-3p, miR-424-5p, miR451, miR-503, miR-503-3p, and miR-503-5p. MiR-21 can be miR-21-5p, miR-22 can be miR-22-3p, and miR-146 can be miR-146b. In some cases, the miRNA can be associated with hypoxia, or can be an hypoxamiR. Non-limiting examples of miRNAs associated with hypoxia include, but are not limited to, miR-21 (e.g., miR-21-5p), miR-22 (e.g., miR-22-3p), miR-27a/b, miR-98, miR-125a, miR-135a, miR-186 (e.g., miR-186-5p), miR-199a-5p, miR-204, miR-210 (e.g., miR-210-3p), and miR-214. In some cases, miR-21 is miR-21-5p. In some cases, miR-22 is miR-22-3p.

The methods of the present disclosure can include measuring or comparing the levels of at least one miRNA. In some embodiments, the level of at least one miRNA is compared to the level of at least another miRNA. For example, the level of an miRNA associated with muscle function can be compared to the level of an miRNA associated with blood oxygen content (e.g., an miRNA associated with erythrocyte function, hypoxia, oxygen tension, oxygen consumption, or oxygen utilization), and/or compared to an miRNA associated with the regulation or expression of BMPR2, ALK1 (or ACVRL1), SMAD9, caveolin 1, and/or KCNK3 genes. In some embodiments, the level of an miRNA associated with blood oxygen content (e.g., an miRNA associated with erythrocyte function, hypoxia, oxygen tension, oxygen consumption, or oxygen utilization) is compared to the level of an miRNA associated with muscle function, and/or the level of an miRNA associated with the regulation or expression of BMPR2, ALK1 (or ACVRL1), SMAD9, caveolin 1, and/or KCNK3 genes. In some embodiments, the level of an miRNA associated with muscle function (e.g., miR-22-3p) is compared to the level of an miRNA associated with erythrocyte function (e.g., miR-451a). In some embodiments, the levels of two or more miRNA associated with muscle function (e.g., miR-22-3p and miR-21-5p) are compared to the levels of a reference RNA in combination with an miRNA associated with erythrocyte function (e.g., Spike-RNA and miR-451a). In some embodiments, a reference RNA is used, such as Spike RNA or UniSp6.

The methods of the present disclosure can include the use of oligonucleotides (or probes) to determine a specific miRNA in a sample (e.g., an miRNA in a sample from a subject with PH). For example, an oligonucleotide can be used for PCR or can be provided in a kit. In some cases, an oligonucleotide can be a polynucleotide probe. An oligonucleotide can be complementary to at least one miRNA associated with blood oxygen content, oxygen tension, consumption, or utilization, erythrocyte function, hypoxia, muscle function, or an miRNA associated with the regulation or expression of BMPR2, ALK1 (or ACVRL1), SMAD9, caveolin 1, and/or KCNK3. The term complementary as used herein in reference to two or more polynucleotides or nucleic acid sequences, refer to polynucleotides (or sequences within one or more polynucleotides) including any nucleic acid sequences that can undergo cumulative base pairing at two or more individual corresponding positions in antiparallel orientation, as in a hybridized duplex. In some cases, complementary can include nucleic acid sequences that have some non-complementary portions, for example when one nucleic acid sequence is longer than the other. In some cases, two nucleic acid sequences are complete complementary to each other (each nucleotide in one of the nucleic acid sequences can undergo a stabilizing base pairing interaction with a nucleotide in the corresponding antiparallel position in the other nucleic acid sequence). In some cases, the oligonucleotide is partially complementary to at least one miRNA. In some embodiments, at least about 20%, but less than 100%, of the residues of the oligonucleotide are complementary to residues in an miRNA sequence. In some embodiments, at least about 50%, but less than 100%, of the residues of the oligonucleotide are complementary to residues in an miRNA sequence. In some embodiments, at least about 70%, at least about 80%, at least about 90%, or at least about 95%, but less than 100%, of the residues of the oligonucleotide are complementary to residues in an miRNA sequence. In some cases, the oligonucleotide is substantially complementary (e.g., at least about 80% complementary) to at least one miRNA. In some embodiments, 100% of the residues of the oligonucleotide are complementary to residues in an miRNA sequence. In some cases, complementary nucleotides can form base pairs with each other, such as the A-T/U and G-C base pairs formed through specific Watson-Crick type hydrogen bonding between the nucleobases of nucleotides and/or polynucleotides at positions antiparallel to each other. The complementarity of other artificial base pairs can be based on other types of hydrogen bonding and/or hydrophobicity of bases and/or shape complementarity between bases.

The oligonucleotide can be complementary to at least a portion of an miRNA associated with erythrocyte function. For example, the oligonucleotide can be complementary to at least a portion of miR-16 (e.g., miR-16-5p), miR-92a, miR-144 (e.g., miR-144-3p), miR-221 (e.g., miR-221-3p), miR-222 (e.g., miR-222-3p), miR-451a, and/or miR-486-5p. In some embodiments, the oligonucleotide is complementary to at least a portion of miR-144 (e.g., miR-144-3p). In some embodiments, the oligonucleotide is complementary to at least a portion of miR-451a.

The oligonucleotide that is complementary to an miRNA associated with muscle function can be complementary to at least a portion of miR-1 (e.g., miR-1-3p), miR-21 (e.g., miR-21-5p), miR-22 (e.g., miR-22-3p), miR-26a, miR-29a (e.g., miR-29a-3p), miR-133b, miR-143-3p, miR-143-5p, miR-146a (e.g., miR-146a-5p), miR-146b-3p, miR-146b-5p, miR-181a (e.g., miR-181a-5p), miR-181b (e.g., miR-181b-5p), miR-181c-5p, miR-181d (e.g., miR-181d-5p), miR-204, miR-206, miR-214 (e.g., miR-214-3p), miR-222 (e.g., miR-222-3p), miR-221 (e.g., miR-221-3p), miR-378 (e.g., miR-378a-3p), miR-424-3p, miR-424-5p, miR-451a, miR-503-3p, and miR-503-5p. In some embodiments, the oligonucleotide that is complementary to an miRNA associated with muscle function can be complementary to at least a portion of miR-135a, miR-186 (e.g., miR-186-5p), miR-210 (e.g., miR-210-3p), and/or miR-204. In some embodiments, the oligonucleotide is complementary to at least a portion of miR-26a, miR-378 (e.g., miR-378a-3p), and/or miR-29a (e.g., miR-29a-3p). In some embodiments, the oligonucleotide is complementary to at least a portion of miR-21 (e.g., miR-21-5p), miR-22 (e.g., miR-22-3p), and/or miR-451. The oligonucleotide can be complementary to at least a portion of miR-21-5p, miR-22-3p, and/or miR-451a. In some embodiments, the oligonucleotide is complementary to at least a portion of miR-21-5p. In some embodiments, the oligonucleotide is complementary to at least a portion of miR-451a. In some embodiments, the oligonucleotide is complementary to at least a portion of miR-22-3p.

The oligonucleotide can be complementary to at least one miRNA associated with hypoxia or can be complementary to an hypoxamiR. The hypoxamiR can be, but is not limited to miR-135a, miR-186 (e.g., miR-186-5p), miR199a-5p, miR-204, miR-210 (e.g., miR-210-3p), or miR-214. The oligonucleotide can be complementary to at least a portion of miR-135a, miR-186 (e.g., miR-186-5p), miR199a-5p, miR-204, miR-210 (e.g., miR-210-3p), and/or miR-214.

The oligonucleotide can be complementary to at least a portion of an miRNA associated with the regulation or expression of BMPR2, ALK1 (or ACVRL1), SMAD9, caveolin 1, and/or KCNK3. In some embodiments, the oligonucleotide is complementary to at least a portion of an miRNA associated with the regulation or expression of BMPR2. In some embodiments, the oligonucleotide is complementary to at least a portion of miR-20 (e.g., miR-20a-5p), miR-21-5p, miR-129-5p, miR-92a-3p, miR-19b-3p, miR-19a-3p, miR-17-5p, miR-100-5p, miR-302c-3p, miR-130a-3p, miR-181c-5p, miR-128-3p, miR-215-5p, miR-7-5p, miR-192-5p, miR-93-5p, miR-26b-5p, miR-93-3p, miR-135a-5p, miR-181a-5p, miR-106b-5p, miR-20b-5p, miR-519d-3p, miR-32-5p, miR-181b-5p, miR-181d-5p, miR-92b-3p, miR-27a-3p, miR-27b-3p, miR-548c-3p, miR-135b-5p, miR-4716-5p, miR-4772-3p, miR-3615, miR-1304-3p, miR-125b-1-3p, miR-6890-3p, miR-6800-3p, miR-6787-3p, miR-6736-3p, miR-4693-5p, miR-103a-2-5p, miR-302d-5p, miR-302b-5p, miR-550b-3p, miR-892c-3p, miR-4676-3p, miR-452-5p, miR-100-3p, miR-490-5p, miR-153-3p, miR-204, miR-135a, miR-375 (e.g., miR-375-3p), and/or miR-494 (e.g., miR-494-3p).

In some embodiments, the oligonucleotide is complementary to at least a portion of an miRNA associated with the regulation or expression of caveolin 1. In some embodiments, the oligonucleotide is complementary to at least a portion of miR-34c-5p, miR-34b-5p, miR-124-3p, miR-103a-3p, miR-7-5p, miR-26b-5p, miR-199a-5p, miR-203a-3p, miR-107, ssc-miR-199a-5p, miR-192-5p, miR-17-5p, miR-20 (e.g., miR-20a-5p), miR-93-5p, miR-106a-5p, miR-194-5p, miR-106b-5p, miR-20b-5p, miR-526b-3p, miR-519d-3p, miR-3609, miR-548ah-5p, miR-4796-3p, miR-3973, miR-873-5p, miR-520h, miR-520g-3p, miR-4463, miR-1238-3p, miR-6749-3p, miR-6792-3p, miR-4691-5p, miR-627-3p, miR-660-3p, miR-5193, miR-670-3p, miR-4277, miR-584-3p, miR-5004-3p, miR-1261, miR-4791, miR-3201, miR-766-5p, miR-3140-3p, miR-4722-5p, miR-4468, miR-4673, miR-4645-5p, miR-4692, miR-4514, miR-4459, miR-556-5p, miR-208b-5p, miR-208a-5p, miR-6165, miR-6753-5p, miR-1911-3p, miR-338-5p, miR-4517, and/or mmu-miR-124-3p.

In some embodiments, the oligonucleotide is complementary to at least a portion of an miRNA associated with the regulation or expression of SMAD9. In some embodiments, the oligonucleotide is complementary to at least a portion of miR-106b-5p, miR-203a-3p, miR-574-5p, miR-653-5p, miR-5585-3p, miR-190a-3p, miR-6867-5p, miR-223-5p, miR-511-3p, miR-5011-5p, miR-1277-5p, miR-665, miR-887-5p, miR-6780a-5p, miR-6779-5p, miR-3689c, miR-3689b-3p, miR-3689a-3p, miR-30b-3p, miR-1273h-5p, miR-6788-5p, miR-30c-2-3p, miR-30c-1-3p, miR-6799-5p, miR-6883-5p, miR-6785-5p, miR-4728-5p, miR-149-3p, miR-7106-5p, miR-7160-5p, miR-4722-5p, miR-6884-5p, miR-485-5p, miR-1827, miR-4649-3p, miR-4768-3p, miR-4478, miR-4419b, miR-3929, miR-940, miR-6893-5p, miR-6808-5p, miR-890, miR-34b-3p, and/or miR-606.

In some embodiments, the oligonucleotide is complementary to at least a portion of an miRNA associated with the regulation or expression of KCNK3. In some embodiments, the oligonucleotide is complementary to at least a portion of miR-6788-5p, miR-30c-2-3p, miR-30c-1-3p, miR-6778-5p, miR-1233-5p, miR-6766-5p, miR-6756-5p, miR-608, miR-4651, miR-7110-5p, miR-6842-5p, miR-6752-5p, miR-6825-5p, miR-6785-5p, miR-6883-5p, miR-4728-5p, miR-8085, miR-149-3p, miR-6731-5p, miR-6878-5p, miR-4763-3p, miR-1207-5p, miR-6722-3p, miR-1909-3p, miR-4707-5p, miR-6732-5p, miR-4296, miR-4322, miR-4265, miR-4417, miR-6816-5p, miR-3196, miR-3180-3p, miR-3180, miR-3656, miR-3621, miR-423-5p, miR-3184-5p, miR-365b-5p, miR-365a-5p, miR-8052, miR-3199, miR-6778-3p, miR-150-5p, miR-6814-5p, and/or miR-3691-3p.

In some embodiments, the oligonucleotide is complementary to at least a portion of an miRNA associated with the regulation or expression of ACVRL1. In some embodiments, the oligonucleotide is complementary to at least a portion of miR-6833-3p, miR-4768-5p, miR-6773-5p, miR-6724-5p, miR-6873-3p, miR-4684-5p, miR-296-5p, miR-942-5p, miR-6817-3p, miR-7110-3p, miR-5088-3p, miR-6756-3p, miR-3127-3p, miR-1237-5p, miR-128-1-5p, miR-128-2-5p, miR-4488, miR-4505, miR-4514, miR-4690-5p, miR-4692, miR-4697-5p, miR-4731-5p, miR-5787, miR-637, miR-6808-5p, miR-6846-5p, miR-6848-5p, miR-6877-5p, miR-6893-5p, miR-940, miR-1224-5p, miR-4751, miR-4753-5p, miR-5004-5p, and/or miR-7160-5p.

The oligonucleotide can be complementary to at least about 80%, at least about 82%, at least about 85%, at least about 87%, at least about 90%, at least about 92%, at least about 95%, at least about 97%, at least about 99%, or about 100% of an miRNA sequence associated with blood oxygen content, or muscle function, or an miRNA associated with the regulation or expression of BMPR2, ALK1 (or ACVRL1), SMAD9, caveolin 1, and/or KCNK3. For example, an oligonucleotide can be complementary to at least 80%, at least 85%, at least 90%, at least 95%, at least 97%, at least 99%, or 100% of a sequence of any miRNA of the present disclosure (e.g., of any one of SEQ ID NOs: 1-80). An miRNA can have a sequence that is at least about 80%, at least about 82%, at least about 85%, at least about 87%, at least about 90%, at least about 92%, at least about 95%, at least about 97%, at least about 99%, or about 100% identical to a sequence of any miRNA of the present disclosure (e.g., to any one of SEQ ID NOs: 1-80). Various methods and software programs can be used to determine the homology, identity, or complementarity between two or more peptides or nucleic acids, such as NCBI BLAST, Clustal W, MAFFT, Clustal Omega, AlignMe, Praline, or another suitable method or algorithm.

TABLE 1
List of miRNAs

| | | SEQ |
|---|---|---|
| | | ID |
| miRNA | Sequence | NO: |
| miR-1-3p | 5′-UGGAAUGUAAAGAAGUAUGUAU | 1 |
| miR-7-5p | 5′-UGGAAGACUAGUGAUUUUGUUGU | 2 |
| miR-16-5p | 5′-UAGCAGCACGUAAAUAUUGGCG | 3 |
| miR-17-5p | 5′-CAAAGUGCUUACAGUGCAGGUAG | 4 |
| miR-19a-3p | 5′-UGUGCAAAUCUAUGCAAAACUGA | 5 |
| miR-19b-3p | 5′-UGUGCAAAUCCAUGCAAAACUGA | 6 |
| miR-20a-5p | 5′-UAAAGUGCUUAUAGUGCAGGUAG | 7 |
| miR-20b-5p | 5′-CAAAGUGCUCAUAGUGCAGGUAG | 8 |
| miR-21-5p | 5′-UAGCUUAUCAGACUGAUGUUGA | 9 |
| miR-22-3p | 5′-AAGCUGCCAGUUGAAGAACUGU | 10 |
| miR-26a-5p | 5′-UUCAAGUAAUCCAGGAUAGGCU | 11 |
| miR-26b-5p | 5′-UUCAAGUAAUUCAGGAUAGGU | 12 |
| miR-27a-3p | 5′-UUCACAGUGGCUAAGUUCCGC | 13 |
| miR-27b-3p | 5′-UUCACAGUGGCUAAGUUCUGC | 14 |
| miR-29a-3p | 5′-UAGCACCAUCUGAAAUCGGUUA | 15 |
| miR-32-5p | 5′-UAUUGCACAUUACUAAGUUGCA | 16 |
| miR-92a-3p | 5′-UAUUGCACUUGUCCCGGCCUGU | 17 |
| miR-92b-3p | 5′-UAUUGCACUCGUCCCGGCCUCC | 18 |
| miR-93-3p | 5′-ACUGCUGAGCUAGCACUUCCCG | 19 |
| miR-93-5p | 5′-CAAAGUGCUGUUCGUGCAGGUAG | 20 |
| miR-100-3p | 5′-CAAGCUUGUAUCUAUAGGUAUG | 21 |
| miR-100-5p | 5′-AACCCGUAGAUCCGAACUUGUG | 22 |
| miR-103a-2-5p | 5′-AGCUUCUUUACAGUGCUGCCUUG | 23 |
| miR-106b-5p | 5′-UAAAGUGCUGACAGUGCAGAU | 24 |
| miR-125b-1-3p | 5′-ACGGGUUAGGCUCUUGGGAGCU | 25 |
| miR-128-3p | 5′-UCACAGUGAACCGGUCUCUUU | 26 |
| miR-129-5p | 5′-CUUUUUGCGGUCUGGGCUUGC | 27 |
| miR-130a-3p | 5′-CAGUGCAAUGUUAAAAGGGCAU | 28 |
| miR-133b | 5′-UUUGGUCCCCUUCAACCAGCUA | 29 |
| miR-135a-5p | 5′-UAUGGCUUUUUAUUCCUAUGUGA | 30 |
| miR-135b-5p | 5′-UAUGGCUUUUCAUUCCUAUGUGA | 31 |
| miR-143-3p | 5′-UGAGAUGAAGCACUGUAGCUC | 32 |
| miR-143-5p | 5′-GGUGCAGUGCUGCAUCUCUGGU | 33 |
| miR-144-3p | 5′-UACAGUAUAGAUGAUGUACU | 34 |
| miR-146a-5p | 5′-UGAGAACUGAAUUCCAUGGGUU | 35 |
| miR-146b-3p | 5′-UGCCCUGUGGACUCAGUUCUGG | 36 |
| miR-146b-5p | 5′-UGAGAACUGAAUUCCAUAGGCU | 37 |
| miR-153-3p | 5′-UUGCAUAGUCACAAAAGUGAUC | 38 |
| miR-181a-5p | 5′-AACAUUCAACGCUGUCGGUGAGU | 39 |
| miR-181b-5p | 5′-AACAUUCAUUGCUGUCGGUGGGU | 40 |
| miR-181c-5p | 5′-AACAUUCAACCUGUCGGUGAGU | 41 |
| miR-181d-5p | 5′-AACAUUCAUUGUUGUCGGUGGGU | 42 |
| miR-186-5p | 5′-CAAAGAAUUCUCCUUUUGGGCU | 43 |
| miR-192-5p | 5′-CUGACCUAUGAAUUGACAGCC | 44 |
| miR-199a-5p | 5′-CCCAGUGUUCAGACUACCUGUUC | 45 |
| miR-204a-5p | 5′-UUCCCUUUGUCAUCCUAUGCCU | 46 |
| miR-206 | 5′-UGGAAUGUAAGGAAGUGUGUGG | 47 |
| miR-210-3p | 5′-CUGUGCGUGUGACAGCGGCUGA | 48 |
| miR-214-3p | 5′-ACAGCAGGCACAGACAGGCAGU | 49 |
| miR-215-5p | 5′-AUGACCUAUGAAUUGACAGAC | 50 |
| miR-221-3p | 5′-AGCUACAUUGUCUGCUGGGUUUC | 51 |
| miR-222-3p | 5′-AGCUACAUCUGGCUACUGGGU | 52 |
| miR-302b-5p | 5′-ACUUUAACAUGGAAGUGCUUUC | 53 |
| miR-302c-3p | 5′-UAAGUGCUUCCAUGUUUCAGUGG | 54 |
| miR-302d-5p | 5′-ACUUUAACAUGGAGGCACUUGC | 55 |
| miR-375-3p | 5′-UUUGUUCGUUCGGCUCGCGUGA | 56 |
| miR-378a-3p | 5′-ACUGGACUUGGAGUCAGAAGG | 57 |
| miR-424-3p | 5′-CAAAACGUGAGGCGCUGCUAU | 58 |
| miR-424-5p | 5′-CAGCAGCAAUUCAUGUUUUGAA | 59 |
| miR-451a | 5′-AAACCGUUACCAUUACUGAGUU | 60 |
| miR-452-5p | 5′-AACUGUUUGCAGAGGAAACUGA | 61 |
| miR-486-5p | 5′-UCCUGUACUGAGCUGCCCCGAG | 62 |
| miR-490-5p | 5′-CCAUGGAUCUCCAGGUGGGU | 63 |
| miR-494-3p | 5′-UGAAACAUACACGGGAAACCUC | 64 |
| miR-503-3p | 5′-GGGGUAUUGUUUCCGCUGCCAGG | 65 |
| miR-503-5p | 5′-UAGCAGCGGGAACAGUUCUGCAG | 66 |
| miR-519d-3p | 5′-CAAAGUGCCUCCCUUUAGAGUG | 67 |
| miR-548c-3p | 5′-CAAAAAUCUCAAUUACUUUUGC | 68 |
| miR-550b-3p | 5′-UCUUACUCCCUCAGGCACUG | 69 |
| miR-892c-3p | 5′-CACUGUUUCCUUUCUGAGUGGA | 70 |
| miR-1304-3p | 5′-UCUCACUGUAGCCUCGAACCCC | 71 |
| miR-3615 | 5′-UCUCUCGGCUCCUCGCGGCUC | 72 |
| miR-4676-3p | 5′-CACUGUUUCACCACUGGCUCUU | 73 |
| miR-4693-5p | 5′-AUACUGUGAAUUUCACUGUCACA | 74 |
| miR-4716-5p | 5′-UCCAUGUUUCCUUCCCCCUUCU | 75 |
| miR-4772-3p | 5′-CCUGCAACUUUGCCUGAUCAGA | 76 |
| miR-6736-3p | 5′-UCAGCUCCUCUCUACCCACAG | 77 |
| miR-6787-3p, | 5′-UCUCAGCUGCUGCCCUCUCCAG | 78 |
| miR-6800-3p | 5′-CACCUCUCCUGGCAUCGCCCC | 79 |
| miR-6890-3p | 5′-CCACUGCCUAUGCCCCACAG | 80 |

The methods of the present disclosure can also include detecting miRNA levels in patients subjected to combination therapy with an active agent used for the treatment of pulmonary hypertension. For example, the methods of the present disclosure can include measuring miRNA levels from a patient subjected to oxygen intake therapy and/or exercise training in combination with an active agent (or drug) used for treating pulmonary hypertension. Non-limiting examples of the active agent include prostanoids, phosphodiesterase inhibitors (e.g., phosphodiesterase-5 (PDE5) inhibitors), endothelin receptor antagonists (ERAs), calcium channel blockers, diuretics, anticoagulants, guanylate cyclase activators, vasodilators, angiotensin-converting-enzyme (ACE) inhibitors, beta blockers, theophyllines, prostacyclins, endothelin receptor antagonists, nitric oxide, and any combination thereof.

Non-limiting examples of prostanoid include beraprost, cicaprost, epoprostenol, iloprost, NS-304, PGE1, prostacyclin, treprostinil, and any combination thereof. Non-limiting examples of the phosphodiesterase-5 (PDE5) inhibitor include sildenafil, tadalafil, vardenafil, and any combination thereof. Non-limiting examples of calcium channel blockers include aryklalkylamines, bepridil, clentiazem, diltiazem, fendiline, gallopamil, mibefradil, prenylamine, semotiadil, terodiline, verapamil, dihydropyridine derivatives, amlodipine, aranidipine, barnidipine, benidipine, cilnidipine, efonidipine, elgodipine, felodipine, isradipine, lacidipine, lercanidipine, manidipine, nicardipine, nifedipine, nilvadipine, nimodipine, nisoldipine, nitrendipine, NZ 105, piperazine derivatives, cinnarizine, dotarizine, flunarizine, lidoflazine, lomerizine, bencyclane, etafenone, fantofarone, monatepil, perhexiline, and any combination thereof. Non-limiting examples of diuretics include organomercurials, chlormerodrin, chlorothiazide, chlorthalidone, meralluride, mercaptomerin sodium, mercumatilin sodium, mercurous chloride, mersalyl, purines, pamabrom, protheobromine, theobromine, steroids, canrenone, oleandrin, spironolactone, sulfonamide derivatives, acetazolamide, ambuside, azosemide, bumetanide, butazolamide, chloraminophenamide, clofenamide, clopamide, clorexolone, disulfamide, ethoxzolamide, furosemide, mefruside, methazolamide, piretanide, torsemide, tripamide, xipamide, thiazides and analogs, althiazide, bendroflumethiazide, benzthiazide, benzylhydrochlorothiazide, buthiazide, chlorthalidone, cyclopenthiazide, cyclothiazide, ethiazide, fenquizone, hydrochlorothiazide, hydroflumethiazide, indapamide, methyclothiazide, metolazone, paraflutizide, polythiazide, quinethazone, teclothiazide, trichlormethiazide, uracils, aminometradine, amiloride, Biogen BG 9719, chlorazanil, ethacrynic acid, etozolin, isosorbide, Kiowa Hakko KW 3902, mannitol, muzolimine, perhexiline, Sanofi-Aventis SR 121463, ticrynafen, triamterene, urea, and any combination thereof. Non-limiting examples of thiazide diuretics include chlorothiazide, chlorthalidone, hydrochlorothiazide, indapamide, metolazone, polythiazide, and any combination thereof. Non-limiting examples of loop diuretics include bumetanide, furosemide, torsemide, and any combination thereof. Non-limiting examples of an anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, pentosan polysulfate, phenindione, phenprocoumon, phosvitin, picotamide, tioclomarol, warfarin, and any combination thereof.

One or more doses of a treatment regimen can be provided to a subject with PH. In some embodiments, dosage levels (e.g., of an active agent) include a maximum dosage of 700 mg/kg and a minimum dosage of 0.5 mg/kg. Dosage levels can include about 0.5, about 1, about 10, about 20, about 25, about 30, about 40, about 50, about 60, about 75, about 80, about 90, about 100, about 110, about 120, about 130, about 140, about 150, about 160, about 170, about 175, about 180, about 190, about 200, about 210, about 220, about 230, about 240, about 250, about 260, about 270, about 275, about 280, about 290, about 300, about 310, about 320, about 330, about 340, about 350, about 360, about 370, about 375, about 380, about 390, about 400, about 410, about 420, about 430, about 440, about 450, about 460, about 470, about 475, about 480, about 490, about 500, about 510, about 520, about 530, about 540, about 550, about 560, about 570, about 575, about 580, about 590, about 600, about 610, about 620, about 630, about 640, about 650, about 660, about 670, about 675, about 680, about 690, and about 700 mg/kg or at least one of these amounts. Dosage levels can include a range from about 0.5 mg/kg to about 700 mg/kg, from about 1 mg/kg to about 700 mg/kg, from about 10 mg/kg to about 700 mg/kg, from about 25 mg/kg to about 700 mg/kg, from about 50 mg/kg to about 700 mg/kg, from about 75 mg/kg to about 700 mg/kg, from about 100 mg/kg to about 700 mg/kg, from about 125 mg/kg to about 700 mg/kg, from about 150 mg/kg to about 700 mg/kg, from about 175 mg/kg to about 700 mg/kg, from about 200 mg/kg to about 700 mg/kg, from about 225 mg/kg to about 700 mg/kg, from about 250 mg/kg to about 700 mg/kg, from about 275 mg/kg to about 700 mg/kg, from about 300 mg/kg to about 700 mg/kg, from about 325 mg/kg to about 700 mg/kg, from about 350 mg/kg to about 700 mg/kg, from about 375 mg/kg to about 700 mg/kg, from about 400 mg/kg to about 700 mg/kg, from about 425 mg/kg to about 700 mg/kg, from about 450 mg/kg to about 700 mg/kg, from about 475 mg/kg to about 700 mg/kg, from about 500 mg/kg to about 700 mg/kg, from about 525 mg/kg to about 700 mg/kg, from about 550 mg/kg to about 700 mg/kg, from about 575 mg/kg to about 700 mg/kg, from about 600 mg/kg to about 700 mg/kg, from about 625 mg/kg to about 700 mg/kg, from about 650 mg/kg to about 700 mg/kg, from about 675 mg/kg to about 700 mg/kg, from about 1 mg/kg to about 600 mg/kg, from about 1 mg/kg to about 500 mg/kg, from about 1 mg/kg to about 400 mg/kg, from about 1 mg/kg to about 300 mg/kg, from about 1 mg/kg to about 200 mg/kg, from about 1 mg/kg to about 100 mg/kg, from about 10 mg/kg to about 700 mg/kg, from about 10 mg/kg to about 600 mg/kg, from about 10 mg/kg to about 500 mg/kg, from about 10 mg/kg to about 400 mg/kg, from about 10 mg/kg to about 300 mg/kg, from about 10 mg/kg to about 200 mg/kg, from about 10 mg/kg to about 100 mg/kg, from about 20 mg/kg to about 700 mg/kg, from about 20 mg/kg to about 600 mg/kg, from about 20 mg/kg to about 500 mg/kg, from about mg/kg 20 to about 400 mg/kg, from about 20 mg/kg to about 300 mg/kg, from about 20 mg/kg to about 200 mg/kg, from about 20 mg/kg to about 150 mg/kg, from about 20 mg/kg to about 100 mg/kg, from about 20 mg/kg to about 75 mg/kg, from about 20 mg/kg to about 50 mg/kg, from about 30 mg/kg to about 700 mg/kg, from about 30 mg/kg to about 60 mg/kg, from about 30 mg/kg to about 500 mg/kg, from about 30 mg/kg to about 400 mg/kg, from about 30 mg/kg to about 300 mg/kg, from about 30 mg/kg to about 200 mg/kg, from about 30 mg/kg to about 100 mg/kg, from about 40 mg/kg to about 700 mg/kg, from about 40 mg/kg to about 600 mg/kg, from about 40 mg/kg to about 500 mg/kg, from about 40 mg/kg to about 400 mg/kg, from about 40 mg/kg to about 300 mg/kg, from about 40 mg/kg to about 200 mg/kg, from about 40 mg/kg to about 100 mg/kg, from about 50 mg/kg to about 700 mg/kg, from about 50 mg/kg to about 600 mg/kg, from about 50 mg/kg to about 500 mg/kg, from about 50 to about 400 mg/kg, from about 50 to about 300 mg/kg, from about 50 to about 200 mg/kg, from about 50 mg/kg to about 100 mg/kg, from about 60 mg/kg to about 700 mg/kg, from about 60 mg/kg to about 600 mg/kg, from about 60 mg/kg to about 500 mg/kg, from about 60 mg/kg to about 400 mg/kg, from about 60 mg/kg to about 300 mg/kg, from about 60 mg/kg to about 200 mg/kg, from about 60 mg/kg to about 100 mg/kg, from about 70 mg/kg to about 700 mg/kg, from about 70 mg/kg to about 600 mg/kg, from about 70 mg/kg to about 500 mg/kg, from about 70 mg/kg to about 400 mg/kg, from about 70 mg/kg to about 300 mg/kg, from about 70 mg/kg to about 200 mg/kg, from about 70 mg/kg to about 100 mg/kg, from about 80 mg/kg to about 700 mg/kg, from about 80 mg/kg to about 600 mg/kg, from about 80 mg/kg to about 500 mg/kg, from about 80 mg/kg to about 400 mg/kg, from about 80 mg/kg to about 300 mg/kg, from about 80 mg/kg to about 200 mg/kg, from about 80 mg/kg to about 100 mg/kg, from about 90 mg/kg to about 700 mg/kg, from about 90 mg/kg to about 600 mg/kg, from about 90 mg/kg to about 500 mg/kg, from about 90 mg/kg to about 400 mg/kg, from about 90 mg/kg to about 300 mg/kg, from about 90 mg/kg to about 200 mg/kg, from about 90 mg/kg to about 100 mg/kg, from about 100 mg/kg to about 700 mg/kg, from about 100 mg/kg to about 600 mg/kg, from about 100 mg/kg to about 500 mg/kg, from about 100 mg/kg to about 400 mg/kg, from about 100 mg/kg to about 300 mg/kg, from about 100 mg/kg to about 200 mg/kg, from about 200 mg/kg to about 700 mg/kg, from about 200 mg/kg to about 600 mg/kg, from about 200 mg/kg to about 500 mg/kg, from about 200 mg/kg to about 400 mg/kg, from about 200 mg/kg to about 300 mg/kg, from about 300 mg/kg to about 700 mg/kg, from about 300 mg/kg to about 600 mg/kg, from about 300 mg/kg to about 500 mg/kg, from about 300 mg/kg to about 400 mg/kg, from about 400 mg/kg to about 700 mg/kg, from about 400 mg/kg to about 600 mg/kg, from about 400 mg/kg to about 500 mg/kg, from about 500 mg/kg to about 700 mg/kg, from about 500 mg/kg to about 600 mg/kg, from about 600 mg/kg to about 700 mg/kg, from about or any range there between.

Multiple doses of one or a combination of drugs can be delivered to a subject with PH periodically, such as one or more times a day, one or more times a week, or one or more times a month, for example. The delivery can be, for example, oral, intravenous, or subcutaneous. A drug can be delivered as a solid or as a liquid and may require the subject to ingest the drug with food or shortly after ingesting food.

The methods of the present disclosure can also be used to determine PH symptoms or improvements of PH symptoms. Non-limiting examples of PH symptoms include breathlessness or shortness of breath (dyspnea), fatigue, dizziness, fainting (syncope), swollen ankles and legs (edema), chest pain, right heart failure, dysfunction, increased blood pressure (e.g., increased blood pressure in the pulmonary vasculature and the right heart), and any combination thereof. Improvements of PH or PH symptoms can be determined by a change in pulmonary vascular resistance (PVR), a change/decrease in blood pressure, a change in cardiac index (CI) (such as mean pulmonary artery pressure (mPAP), mean right atrial pressure (mRAP), mixed venous oxygen saturation (SvO2), and right ventricular cardiac power), a change in clinical measures of symptoms and function, including but not limited to submaximal exercise (6-minute walk test (6 MWT)), heart rate recovery (HRR) after the 6 MWT, the Borg dyspnea index (BDI), WHO Functional Class, N-terminal pro-brain natriuretic peptide, measurements of the right-heart stress markers B-type natriuretic peptide biomarkers, and/or quality of life by the SF-36 (Registered trademark) Health Survey. Presence of PH can be determined based on a biomarker (e.g., miRNA), a genetic testing or for example, the measurement of the right-heart stress markers B-type natriuretic peptide biomarkers.

The Borg dyspnea index (BDI) is a numerical scale for assessing perceived dyspnea (breathing discomfort). BDI measures the degree of breathlessness after completion of the 6 minute walk test (6 MWT), where a BDI of 0 indicates no breathlessness and 10 indicates maximum breathlessness. The BDI of a subject can be decreased by at least about 0.2, 0.3, 0.4, 0.5, 0.6, 0.7, 0.8, 0.9, 1, 1.5, 2, 2.5, 3, 3.5, 4, 4.5, 5 or more index points (e.g., BDI can decrease after treatment intervention). The BDI of a subject can be decreased by at least about 1 index point. The PVR can be determined by right heart catheterization, and cardiac function can be determined by echocardiography or cardiac hemodynamic data.

The methods of the present disclosure can also include measuring the walking capacity (or walking distance) of a subject. The walking capacity of a subject can be determined using a 6-minute walk test (6 MWT). The walking capacity can be measured prior to and/or after therapy (e.g., prior to exercise therapy, prior to oxygen intake, after exercise therapy, after oxygen intake, prior to treatment with combination therapy, after treatment with combination therapy, prior to drug intake, or after drug intake). Treatment with oxygen can include nightly oxygen intake, daily oxygen intake, hourly oxygen intake, or any other treatment involving any level of oxygen intake. In some embodiments, the walking capacity (or walking distance) of a subject is improved after exercise and/or after oxygen intake therapy compared to the walking capacity of the same subject prior to exercise and/or prior to oxygen intake therapy. The walking capacity can be measured at multiple time points and can also be used for managing disease progression. For example, the walking capacity can be measured for the same subject at different time points, or the walking capacity of a subject with PH (or at risk of developing PH) can be compared to the walking capacity of a healthy subject or a subject without PH, or can be compared to a reference or a control. The subject can be a subject with pulmonary hypertension, at risk of developing pulmonary hypertension, or a subject showing signs or symptoms of PH. Alternatively, the subject can be a healthy subject or a subject without pulmonary hypertension. The subject can also be a group of subjects (e.g., subjects with PH). The walking capacity can be measured prior to treatment, after treatment, or both. The walking capacity can be measured hourly, every two hours, every six hours, every twelve hours, every eighteen hours, daily, twice a day, three times a day, every other day, weekly, twice a week, three times a week, four times a week, every other week, monthly, every other month, every six months, twice a year, or yearly. The walking capacity can be improved by at least about 5 meters, by at least about 10 meters, by at least about 15 meters, by at least about 20 meters, by at least about 25 meters, by at least about 30 meters, by at least about 35 meters, by at least about 40 meters, by at least about 45 meters, by at least about 50 meters, by at least about 55 meters, by at least about 60 meters, by at least about 65 meters, by at least about 70 meters, by at least about 75 meters, by at least about 80 meters, by at least about 85 meters, by at least about 90 meters, by at least about 95 meters, by at least about 100 meters, by at least about 110 meters, by at least about 120 meters, by at least about 130 meters, by at least about 140 meters, by at least about 150 meters, by at least about 160 meters, by at least about 170 meters, by at least about 180 meters, by at least about 190 meters, by at least about 200 meters, by at least about 210 meters, by at least about 220 meters, by at least about 230 meters, by at least about 240 meters, by at least about 250 meters, by at least about 275 meters, by at least about 300 meters, by at least about 350 meters, by at least about 500 meters, by at least about 750 meters, by at least about 800 meters, by at least about 850 meters, by at least about 900 meters, by at least about 1000 meters, or more. The walking capacity can also be improved by at least about, 10%, by at least about 20%, by at least about 30%, by at least about 40%, by at least about 50%, by at least about 60%, by at least about 70%, by at least about 80%, by at least about 90%, by at least about 1-fold, by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, or more. The walking capacity can be improved by about 10%, about 20%, about 30%, about 40%, about 50%, about 60%, about 70%, about 80%, about 90%, about 1-fold, about 2-fold, about 3-fold, about 4-fold, about 5-fold, about 6-fold, about 7-fold, about 8-fold, about 9-fold, about 10-fold, or more. The 6-minute walking distance can be improved after about one day, after about two days, after about three days, after about four days, after about five days, after about six days, after about seven days, after about one week, after about two weeks, after about three weeks, after about four weeks, after about five weeks, after about six weeks, after about seven weeks, after about eight weeks, after about one month, after about two months, after about three months, after about four months, after about five months, after about six months, after about seven months, after about eight months, after about nine months, after about ten months, after about eleven months, after about twelve months, after about one year, after about two years, after about three years, after about four years, after about five years, or more. In some cases, the 6-minute walking distance can be improved in subjects who undergo exercise training and/or oxygen intake (e.g., nightly oxygen intake) treatment. In some cases, the distance walked in the 6-minute walking distance test can be longer in subjects who are being treated for PH (e.g., drug therapy, exercise, and/or oxygen therapy). In some cases, the walking distance is improved when comparing the distance walked during the 6-minute walking distance at baseline (first measure) compared to the distance walked after the subject starts or receives treatment (e.g., after oxygen intake therapy, exercise training, and/or drug treatment).

Hemodynamics can be improved after about one day, after about two days, after about three days, after about four days, after about five days, after about six days, after about one week, after about two weeks, after about three weeks, after about four weeks, after about five weeks, after about six weeks, after about seven weeks, after about eight weeks, after about one month, after about two months, after about three months, after about four months, after about five months, after about six months, after about seven months, after about eight months, after about nine months, after about ten months, after about eleven months, after about twelve months, after about one year, after about two years, after about three years, after about four years, after about five years, or more, after a patient is subjected to exercise training, oxygen intake (e.g., nightly oxygen intake), and/or drug treatment.

The methods of the present disclosure can also include measuring the pulmonary arterial pressure (PAP), the pulmonary vascular resistance (PVR), the pulmonary capillary wedge pressure (PCWP), and/or the left ventricle end diastolic pressure (LVEDP) in a subject. The PAP, PVR, PCWP, and/or LVEDP can be lowered by at least about 1 mmHg, by at least about 2 mmHg, by at least about 3 mmHg, by at least about 4 mmHg, by at least about 5 mmHg, by at least about 6 mmHg, by at least about 7 mmHg, by at least about 8 mmHg, by at least about 9 mmHg, by at least about 10 mmHg, by at least about 12 mmHg, by at least about 15 mmHg, by at least about 18 mmHg, by at least about 20 mmHg, by at least about 25 mmHg, by at least about 30 mmHg, by at least about 35 mmHg, by at least about 40 mmHg, by at least about 45 mmHg, by at least about 50 mmHg, by at least about 55 mmHg, by at least about 60 mmHg, or more, in a subject. For example, a subject identified as having PH can show a decrease in PAP of about 1 mmHg, of about 2 mmHg, of about 3 mmHg, of about 4 mmHg, of about 5 mmHg, of about 6 mmHg, of about 7 mmHg, of about 8 mmHg, of about 9 mmHg, of about 10 mmHg, of about 12 mmHg, of about 15 mmHg, of about 18 mmHg, of about 20 mmHg, of about 25 mmHg, of about 30 mmHg, of about 35 mmHg, of about 40 mmHg, of about 45 mmHg, of about 50 mmHg, or more, after the subject undergoes intervention (e.g., after a subject is treated with an exercise regimen or oxygen intake). The PAP, PVR, PCWP, and/or LVEDP can be measured before and after treatment. The PAP, PVR, PCWP, and/or LVEDP can be measured hourly, every two hours, every five hours, every ten hours, every twelve hours, daily, every other day, twice a day, three times a day, four times a day, once a week, twice a week, three times a week, five times a week, every other week, every month, every other month, every six months, every year, or longer. In some cases, the PAP, PVR, PCWP, and/or LVEDP is measured before and/or after exercise therapy. In some embodiments, the PAP, PVR, PCWP, and/or LVEDP is measured before and/or after oxygen intake therapy. PAP, PVR, PCWP, and/or LVEDP can be reduced in a subject. For example, the PAP, PVR, PCWP, and/or LVEDP can be reduced in a subject after treatment (e.g., after exercise and/or nightly oxygen intake therapy). In some cases, the PAP, PVR, PCWP, and/or LVEDP can be reduced in a subject after treatment (after treatment starts) as compared to before treatment or before the commencement of treatment. Treatment can include exercise, oxygen intake (e.g., nightly oxygen intake), combination therapy, drug intake, and any combination thereof. In some cases, the PAP, PVR, PCWP, and/or LVEDP of a subject is measured at different time points in the same subject. In some cases, the PAP, PVR, PCWP, and/or LVEDP of a subject is compared to that of a healthy subject. In some cases, the PAP, PVR, PCWP, and/or LVEDP of a subject is compared to that of a subject without pulmonary hypertension. In some cases, the PAP, PVR, PCWP, and/or LVEDP of a subject is compared to a reference or a control. In some cases, the reference or control can be predetermined. In some cases, the reference or control can be a fixed value. In some cases, the reference or control can be an average of the PAP, PVR, PCWP, and/or LVEDP obtained from subjects with PH. In some cases, the reference or control can be an average of the PAP, PVR, PCWP, and/or LVEDP obtained from a healthy subject or obtained from a subject without pulmonary hypertension. The PAP, PVR, PCWP, and/or LVEDP can be measured by a catheter. The mean PAP can be lowered in a subject by at least about 1 mmHg, by at least about 2 mmHg, by at least about 3 mmHg, by at least about 4 mmHg, by at least about 5 mmHg, by at least about 6 mmHg, by at least about 7 mmHg, by at least about 8 mmHg, by at least about 9 mmHg, by at least about 10 mmHg, by at least about 12 mmHg, by at least about 15 mmHg, by at least about 18 mmHg, by at least about 20 mmHg, by at least about 25 mmHg, by at least about 30 mmHg, by at least about 35 mmHg, by at least about 40 mmHg, by at least about 45 mmHg, by at least about 50 mmHg, by at least about 55 mmHg, by at least about 60 mmHg, or more (e.g., versus baseline, or when comparing before and after therapy intervention). The PAP of a subject can be at least about 10 mmHg at rest, at least about 15 mmHg at rest, at least about 20 mmHg at rest, at least about 25 mmHg at rest, at least about 30 mmHg at rest, at least about 35 mmHg at rest, at least about 40 mmHg at rest, at least about 45 mmHg at rest, at least about 50 mmHg at rest, at least about 55 mmHg at rest, at least about 75 mmHg at rest, at least about 80 mmHg at rest, at least about 90 mmHg at rest, at least about 100 mmHg at rest, or more than at least about 100 mmHg at rest. In some cases, the PAP of a subject is not less than about 20 mmHg, not less than about 25 mmHg, not less than about 30 mmHg, not less than about 35 mmHg, not less than about 40 mmHg, not less than about 45 mmHg, not less than about 50 mmHg, not less than about 75 mmHg, not less than about 80 mmHg, not less than about 90 mmHg, or not less than about 100 mmHg (e.g., while exercising or immediately after exercising). The PAP of a subject can be from about 25 mmHg to about 35 mmHg, from about 25 mmHg to about 50 mmHg, from about 25 mmHg to about 75 mmHg, from about 25 mmHg to about 100 mmHg, from about 35 mmHg to about 50 mmHg, from about 50 mmHg to about 75 mmHg, from about 50 mmHg to about 100 mmHg, or from about 50 mmHg to about 150 mmHg. In some embodiments, the pulmonary vascular resistance (PVR) of a subject is not less than about 3 mmHg/liter/minute, not less than about 5 mmHg/liter/minute, not less than about 7 mmHg/liter/minute, or not less than about 10 mmHg/liter/minute. In some embodiments, the pulmonary capillary wedge pressure (PCWP) and/or left ventricle end diastolic pressure (LVEDP) is not less than about 5 mmHg, not less than about 7 mmHg, not less than about 10 mmHg, not less than about 15 mmHg, not less than about 20 mmHg, not less than about 30 mmHg, not less than about 40 mmHg, not less than about 50 mmHg, or not less than about 70 mmHg. In some embodiments, the pulmonary capillary wedge pressure (PCWP) and/or left ventricle end diastolic pressure (LVEDP) is not greater than about 5 mmHg, not greater than about 7 mmHg, not greater than about 10 mmHg, not greater than about 15 mmHg, not greater than about 20 mmHg, not greater than about 30 mmHg, not greater than about 40 mmHg, not greater than about 50 mmHg, or not greater than about 70 mmHg. In some embodiments, the pulmonary capillary wedge pressure (PCWP) and/or left ventricle end diastolic pressure (LVEDP) is less than about 15 mmHg. The pulmonary capillary wedge pressure (PCWP) and/or left ventricle end diastolic pressure (LVEDP) can be from about 1 mmHg to about 50 mmHg, from about 3 mmHg to about 75 mmHg, from about 7 mmHg to about 20 mmHg, from about 12 mmHg to about 15 mmHg, or from about 6 mmHg to about 15 mmHg. A reference PAP or PAP from a healthy subject or a subject without pulmonary hypertension can be about 10 mmHg, about 12 mmHg, about 15 mmHg, about 20 mmHg, about 25 mmHg, about 30 mmHg, or about 35 mmHg. In some cases, the reference PAP or PAP from a healthy subject or a subject without pulmonary hypertension is less than about 10 mmHg, less than about 12 mmHg, less than about 15 mmHg, less than about 20 mmHg, less than about 25 mmHg, less than about 30 mmHg, or less than about 35 mmHg.

Methods of the present disclosure can include providing oxygen therapy (e.g., nightly oxygen) to a subject having pulmonary hypertension, suspected of having pulmonary hypertension, or at risk of developing pulmonary hypertension. Oxygen (e.g., supplemental oxygen) can be provided as part of an oxygen therapy, such as nightly oxygen intake, daily oxygen intake, hourly oxygen intake, or monthly oxygen intake. Oxygen therapy can include the delivery of supplemental oxygen by any method. In some cases, oxygen therapy including oxygen enriched gas or pure oxygen can be delivered to the airway of a subject with PH. For non-ambulatory subjects, or for use during non-ambulatory periods, oxygen can be provided, for example, from a stationary oxygen concentrator, which can use of a pressure swing adsorption system to generate the oxygen. In some cases, oxygen concentrators utilizing pressure swing adsorption (“PSA”) systems, containing approximately 21% oxygen, can be used to process ambient air by separating oxygen from the ambient air. Thus, a subject can be supplied with gas containing higher concentrations of oxygen. Oxygen therapy can be delivered via oxygen gas, liquid oxygen, oxygen concentrators or hyperbaric oxygen therapy. In some cases, compressed oxygen systems can be used, such as in cases where oxygen is not needed all the time (e.g., oxygen is needed when walking or performing physical activity). In some cases, oxygen therapy is provided by a liquid oxygen system. In some embodiments, a nose mask is used to provide oxygen. For example, a face mask can be used to provide oxygen from about 5 to about 8 liters per minute (LPM) with a concentration of oxygen delivered from about 28% to about 50%. In some cases, a partial rebreathing mask can be used, which is based on a simple mask, but featuring a reservoir bag, which can increase the provided oxygen concentration to about 40-70% oxygen at about 5-15 LPM. Non-rebreather masks can draw oxygen from attached reservoir bags, with one-way valves that direct exhaled air out of the mask. Non-rebreather masks can deliver close to about 100% oxygen when properly fitted and used at flow rates of 8-10 LPM or higher. This type of mask can be used for acute medical emergencies. In some cases, a nasal cannula is used to provide oxygen. For example, a nasal cannula can be used to provide oxygen at low flow rates, from about 2 to about 6 liters per minute, delivering a concentration of between about 24% to about 40%. Oxygen therapy can be short-term or long-term therapy. A variety of oxygen concentrations can be provided to a subject with PH. Supplemental oxygen can include an FIO₂ (fraction of inspired oxygen) greater than the 21% oxygen in room (ambient) air. Oxygen concentration can be delivered at anywhere from about 2% to about 100% of oxygen by volume. A subject with PH can be supplied with about 2%, about 5%, about 10%, about 15%, about 20%, about 21%, about 22%, about 23%, about 24%, about 25%, about 26%, about 27%, about 28%, about 29%, about 30%, about 35%, about 40%, about 45%, about 50%, about 55%, about 60%, about 65%, about 70%, about 75%, about 80%, about 85%, about 90%, about 95%, or about 100% oxygen. A subject with PH can be supplied with at least about 2%, at least about 5%, at least about 10%, at least about 15%, at least about 20%, at least about 21%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% oxygen. A subject with PH can be supplied with at most about 2%, at most about 5%, at most about 10%, at most about 15%, at most about 20%, at most about 21%, at most about 22%, at most about 23%, at most about 24%, at most about 25%, at most about 26%, at most about 27%, at most about 28%, at most about 29%, at most about 30%, at most about 35%, at most about 40%, at most about 45%, at most about 50%, at most about 55%, at most about 60%, at most about 65%, at most about 70%, at most about 75%, at most about 80%, at most about 85%, at most about 90%, at most about 95%, or at most about 100% oxygen. Oxygen supplied to a subject can range, for example, from about 5%-36%, from about 5%-40%, from about 5%-100%, from about 15%-60, from about 15%-40%, from about 15%-80%, from about 15%-100%, from about 20%-40%, from about 20%-60%, from about 20%-90%, or from about 20%-100%. Oxygen can be provided at different flow rates.

In some cases, the subject is given oxygen therapy during the night. In some cases, the subject is given oxygen therapy during the day. In some cases, the subject is given oxygen therapy for a few hours during the day and/or a few hours during the night. Oxygen can be administered every hour, every 30 minutes, every other hour, every 2 hours, every 3 hours, every 4 hours, every 6 hours, every 8 hours, every 10 hours, every 12 hours, every 15 hours, every 24 hours, every 30 hours, every day, every other day, every two days, every three days, every four days, once a week, twice a week, three times a week, four times a week, five times a week, six times a week, seven times a week, once a month, twice a month, three times a month, four times a month, six times a month, or eight times a month. Oxygen can be administered to a subject for about 30 minutes, for about 1 hour, for about 2 hours, for about 3 hours, for about 4 hours, for about 5 hours, for about 6 hours, for about 7 hours, for about 8 hours, for about 9 hours, for about 10 hours, for about 12 hours, for about 15 hours, for about 18 hours, for about 20 hours, or for about 24 hours per day, or per night, or per day administered, or per night administered, or per time administered, or per week, or per month. For example, nightly oxygen intake or daily oxygen intake can be for about 30 minutes, for about 1 hour, for about 2 hours, for about 3 hours, for about 4 hours, for about 5 hours, for about 6 hours, for about 7 hours, for about 8 hours, for about 9 hours, for about 10 hours, for about 12 hours, for about 15 hours, for about 20 hours, or for about 24 hours. Nightly oxygen intake or daily oxygen intake can be from about 30 minutes to about 2 hours, from about 30 minutes to about 1 hour, from about 30 minutes to about 3 hours, from about 30 minutes to about 5 hours, from about 30 minutes to about 8 hours, from about 30 minutes to about 10 hours, from about 30 minutes to about 15 hours, from about 30 minutes to about 18 hours, from about 30 minutes to about 22 hours, from about 30 minutes to about 24 hours, from about 1 hour to about 2 hours, from about 1 hour to about 3 hours, from about 1 hour to about 4 hours, from about 1 hour to about 5 hours, from about 1 hour to about 6 hours, from about 1 hour to about 7 hours, from about 1 hour to about 8 hours, from about 1 hour to about 9 hours, from about 1 hour to about 10 hours, from about 1 hour to about 11 hours, from about 1 hour to about 12 hours, from about 1 hour to about 14 hours, from about 1 hour to about 16 hours, from about 1 hour to about 18 hours, from about 1 hour to about 20 hours, from about 1 hour to about 22 hours, from about 1 hour to about 24 hours, from about 2 hours to about 3 hours, from about 2 hours to about 4 hours, from about 2 hours to about 5 hours, from about 2 hours to about 6 hours, from about 2 hours to about 7 hours, from about 2 hours to about 8 hours, from about 2 hours to about 9 hours, from about 2 hours to about 10 hours, from about 2 hours to about 12 hours, from about 2 hours to about 14 hours, from about 2 hours to about 16 hours, from about 2 hours to about 18 hours, from about 2 hours to about 20 hours, from about 2 hours to about 22 hours, from about 2 hours to about 24 hours, from about 3 hours to about 4 hours, from about 3 hours to about 5 hours, from about 3 hours to about 6 hours, from about 3 hours to about 8 hours, from about 3 hours to about 10 hours, from about 3 hours to about 14 hours, from about 3 hours to about 16 hours, from about 3 hours to about 18 hours, from about 3 hours to about 20 hours, from about 3 hours to about 23 hours, from about 3 hours to about 24 hours, from about 4 hours to about 5 hours, from about 4 hours to about 8 hours, from about 4 hours to about 10 hours, from about 4 hours to about 12 hours, from about 4 hours to about 15 hours, from about 4 hours to about 20 hours, from about 5 hours to about 8 hours, from about 5 hours to about 10 hours, from about 5 hours to about 12 hours, from about 6 hours to about 8 hours, from about 6 hours to about 10 hours, from about 6 hours to about 12 hours, from about 6 hours to about 15 hours, from about 6 hours to about 18 hours, from about 6 hours to about 24 hours per day/night, or per day/night administered, or per time administered, or per week, or per month.

Methods of the present disclosure can include subjecting a patient (or subject) having pulmonary hypertension, suspected of having pulmonary hypertension, or at risk of developing pulmonary hypertension to exercise training. Exercise training can be monitored by a physician, and/or a medical professional, and/or a personal trainer, and/or a supervisor. Exercise training can also be performed by a subject without monitoring. Exercise training can include cardio-based exercise and/or weight training. Non-limiting examples of exercise training include walking, running, stair exercises (e.g., stair climbing), elliptical training, biking, and any combination thereof. In some embodiments, exercise training is walking. In some embodiments, exercise training includes measuring the distance walked by a subject (e.g., distance walked during a 6 minute walking distance test). Exercise training can be for about 5 minutes, for about 10 minutes, for about 15 minutes, for about 20 minutes, for about 30 minutes, for about 40 minutes, for about 45 minutes, for about 1 hour, for about 1.5 hours, for about 2 hours, for about 2.5 hours, for about 3 hours, for about 3.5 hours, for about 4 hours, for about 6 hours, for about 8 hours, for about 10 hours, for about 14 hours, for about 16 hours, for about 20 hours, for about 22 hours, or more, per day, or per week, or per day that exercise training is performed, or per exercise training session. Exercise training can be for at least about 5 minutes, for at least about 10 minutes, for at least about 15 minutes, for at least about 20 minutes, for at least about 30 minutes, for at least about 40 minutes, for at least about 45 minutes, for at least about 1 hour, for at least about 1.5 hours, for at least about 2 hours, for at least about 2.5 hours, for at least about 3 hours, for at least about 3.5 hours, for at least about 4 hours, for at least about 6 hours, for at least about 8 hours, for at least about 10 hours, for at least about 14 hours, for at least about 16 hours, for at least about 20 hours, for at least about 22 hours, or more, per day, or per week, or per day that exercise training is performed, or per exercise training session. Exercise training can be for at most about 5 minutes, for at most about 10 minutes, for at most about 15 minutes, for at most about 20 minutes, for at most about 30 minutes, for at most about 40 minutes, for at most about 45 minutes, for at most about 1 hour, for at most about 1.5 hours, for at most about 2 hours, for at most about 2.5 hours, for at most about 3 hours, for at most about 3.5 hours, for at most about 4 hours, for at most about 6 hours, for at most about 8 hours, for at most about 10 hours, for at most about 14 hours, for at most about 16 hours, for at most about 20 hours, for at most about 22 hours, or more, per day, or per week, or per day that exercise training is performed, or per exercise training session. Exercise training can be performed about once a day, about twice a day, about three times a day, about four times a day, about five times a day, about every day, about every other day, about every three days, about once a week, about twice a week, about three times a week, about four times a week, about five times a week, about six times a week, about seven times a week, about once a month, about twice a month, about three times a month, about five times a month, about ten times a month, about twenty times a month, about thirty times a month, about every two months, about every three months, about every six months, about once a year, about twice a year, about four times a year, about six times a year, about twelve times a year, about twenty four times a year, about 50 times a year, about 100 times a year, or more. Exercise training can be performed at least about once a day, at least about twice a day, at least about three times a day, at least about four times a day, at least about five times a day, at least about every day, at least about every other day, at least about every three days, at least about once a week, at least about twice a week, at least about three times a week, at least about four times a week, at least about five times a week, at least about six times a week, at least about seven times a week, at least about once a month, at least about twice a month, at least about three times a month, at least about five times a month, at least about ten times a month, at least about twenty times a month, at least about thirty times a month, at least about every two months, at least about every three months, at least about every six months, at least about once a year, at least about twice a year, at least about four times a year, at least about six times a year, at least about twelve times a year, at least about twenty four times a year, at least about 50 times a year, at least about 100 times a year, or more. Exercise training can be performed at most about once a day, at most about twice a day, at most about three times a day, at most about four times a day, at most about five times a day, at most about every day, at most about every other day, at most about every three days, at most about once a week, at most about twice a week, at most about three times a week, at most about four times a week, at most about five times a week, at most about six times a week, at most about seven times a week, at most about once a month, at most about twice a month, at most about three times a month, at most about five times a month, at most about ten times a month, at most about twenty times a month, at most about thirty times a month, at most about every two months, at most about every three months, at most about every six months, at most about once a year, at most about twice a year, at most about four times a year, at most about six times a year, at most about twelve times a year, at most about twenty four times a year, at most about 50 times a year, or at most about 100 times a year.

The methods of the present disclosure can be used to increase or improve heart function, right heart function, and/or left heart function. The methods of the present disclosure can also be used to increase or improve exercise capacity of a subject. The methods of the present disclosure can be used to reduce pulmonary vascular resistance in a subject. In some embodiments, heart function is improved after exercise training and/or oxygen intake (e.g., nightly oxygen intake). In some embodiments, exercise capacity is improved after exercise training and/or oxygen intake (e.g., nightly oxygen intake). In some embodiments, pulmonary vascular resistance is reduced after exercise training and/or oxygen intake therapy (e.g., nightly oxygen intake). Heart function, oxygen intake, and/or exercise capacity of a subject can improve after one session, two sessions, three sessions, five sessions, one day, two days, three days, four days, seven days, two weeks, three weeks, one month, two months, four months, six months, seven months, eight months, one year, two years, three years, four years, or more after the subject start exercise training and/or oxygen intake therapy (e.g., nightly oxygen intake). Pulmonary vascular resistance can be reduced after one session, two sessions, three sessions, five sessions, one day, two days, three days, four days, seven days, two weeks, three weeks, one month, two months, four months, six months, 8 months, one year, two years, three years, four years, or more after the subject start exercise training and/or oxygen intake therapy (e.g., nightly oxygen intake).

The methods of the present disclosure can include determining a predetermined set of miRNAs by using RT-PCR/qRT-PCR (real time polymerase chain reaction). The workflow for RT-PCR/qRT-PCR can include the following steps: (i) extracting the total RNA from a sample, e.g. biological sample, blood, whole blood, serum, or plasma, of a subject, e.g. a human subject with unknown clinical condition, e.g. healthy person or patient suffering from a disease (e.g. pulmonary hypertension), and obtaining cDNA samples by an RNA reverse transcription (RT) reaction using miRNA-specific primers; or collecting a biological sample, e.g. blood, whole blood, serum, or plasma, from a human and conducting reverse transcriptase reaction using miRNA-specific primers with the biological sample, e.g. blood, whole blood, serum, or plasma, being a buffer so as to prepare cDNA samples, (ii) designing miRNA-specific cDNA forward primers and providing universal reverse primers to amplify the cDNA via polymerase chain reaction (PCR), (iii) adding a probe, e.g. labeled or fluorescent probe, to conduct PCR, and (iv) detecting and comparing the variation in levels of miRNAs in the biological sample, relative to the miRNAs in a control (e.g., healthy subject) biological sample or relative to a biological sample obtained from the same subject but at a different time point.

The methods of the present disclosure can also include amplifying nucleic acids (e.g., a plurality of nucleic acids, miRNAs, RNAs, DNAs, or cDNAs). Any type of nucleic acid amplification reaction can be used to amplify a target nucleic acid and generate an amplified product. Non-limiting examples of nucleic acid amplification methods of the present disclosure include reverse transcription, primer extension, polymerase chain reaction (PCR), ligase chain reaction, helicase-dependent amplification, asymmetric amplification, rolling circle amplification, and multiple displacement amplification (MDA). The PCR can be, for example, a quantitative PCR (qPCR), a real-time PCR (RT-PCR), or RT-qPCR. In cases where a target ribonucleic acid (RNA) is amplified, deoxyribonucleic acid (DNA) can be obtained by reverse transcription of the RNA and subsequent amplification of the DNA can be used to generate an amplified DNA product. In some embodiments, the RNA is miRNA. In some embodiments, the DNA is complementary DNA (cDNA). The amplified DNA product can be indicative of the presence of the target RNA in a biological sample. In cases where DNA is amplified, any DNA amplification can be employed. Non-limiting examples of DNA amplification methods include polymerase chain reaction (PCR), variants of PCR (e.g., real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, touchdown PCR), and ligase chain reaction (LCR). In some cases, DNA amplification is linear. In some cases, DNA amplification is exponential. In some cases, DNA amplification is achieved with nested PCR, which can improve sensitivity of detecting amplified DNA products. In some cases, RNA or DNA is produced by nucleic acid amplification assays including polymer chain reaction, nested polymer chain reaction, and rolling circle amplification. Moreover, amplification of a nucleic acid can be linear, circular, exponential, or a combination thereof. Amplification can be emulsion based or can be non-emulsion based.

In some embodiments, a DNA polymerase is added to a nucleic acid amplification reaction. Non-limiting examples of DNA polymerase include bacteriophage φ29 (phi29) DNA polymerase, Bst large fragment DNA polymerase (Exo(−) Bst and exo(−)Bca DNA polymerase), phage M2 DNA polymerase, phage φPRD1 DNA polymerase, exo(−)VENT® DNA polymerase, Klenow fragment of DNA polymerase I, T5 DNA polymerase, Sequenase, PRD1 DNA polymerase, and T4 DNA polymerase holoenzyme.

The methods of the present disclosure can include amplifying nucleic acid molecules via rolling circle amplification. The rolling circle amplification of the present disclosure can use a padlock probe (e.g., linear DNA backbone) that recognizes an miRNA, a DNA ligase (e.g., T7 ligase or SplintR ligase), and a DNA polymerase (e.g., phi29 polymerase) to produce a DNA product containing multiple copies of the miRNA sequence. In some embodiments, the DNA ligase is a SplintR ligase. In some embodiments, the DNA polymerase is a phi29 polymerase.

The rolling circle amplification of the present disclosure can be performed by a ligation reaction followed by an amplification reaction. The rolling circle amplification of the present disclosure can use a commercially available kit for the amplification reaction (e.g., Illustra™ Ready-To-Go™ GenomiPhi™ V3 DNA Amplification Kit from GE Healthcare). The kit can be used to amplify the whole genome. The kit can contain a freeze-dried cake that contains DNA polymerase (e.g., phi29 polymerase), nucleotides, salts, buffers, and/or random hexamers (e.g., random hexamer primers). In some embodiments, the kit does not include random hexamers. In some embodiments, the rolling circle amplification does not include random hexamers. The rolling circle amplification of the present disclosure can use a circularized probe, a padlock probe, and/or any other probe. In some embodiments, the any other probe is an open circle probe. In some embodiments, a probe is an open circle probe. In some embodiments, the any other probe is a closed circle probe. In some embodiments, a probe is a closed circle probe. In some embodiments, a probe or a padlock probe comprises a sequence that is identical to the sequence of a circularized probe. In some embodiments, a probe comprises an identical sequence to a circularized probe but without being circularized. In some embodiments, a probe is circularized. In some embodiments, a probe is not circularized. A circularized probe can be a mixture of circularized probes. In some embodiments, the mixture contains circularized probes specific for one or more miRNAs. In some embodiments, the circularized probe is a random circularized probe. In some embodiments, the circularized probe contains a sequence specific for an miRNA. In some embodiments, the circularized probe contains a sequence that is complementary to an miRNA (or more than one miRNA). A circularized probe or the padlock probe of the present disclosure can include a sequence that is complementary to an miRNA and a probe sequence. A circularized probe of the present disclosure can include a sequence that is a reverse complementary sequence to an miRNA (or a portion of an miRNA) and a probe sequence. A circularized probe or the padlock probe of the present disclosure can include a sequence that is a reverse complementary sequence to an miRNA of the present disclosure (e.g., an miRNA disclosed in the present disclosure, an miRNA associated with PH, an miRNAs associated with muscle function, blood oxygen content, oxygen tension, consumption, utilization, erythrocyte function, hypoxia, or miRNAs associated with the regulation or expression of BMPR2, ACVRL1, SMAD9, caveolin 1, and/or KCNK3 genes). A circularized probe of the present disclosure can include a sequence that is at least partially reverse complementary to an miRNA associated with PH. The complementary or reverse complementary sequence of the circularized probe or of the padlock probe of the present disclosure can be about 1-2 bases, about 1-5 bases, about 1-10 bases, about 1-15 bases, about 1-25 bases, 1-30 bases, about 1-35 bases, about 10-40 bases, or more.

The complementary or reverse complementary sequence of the circularized probe or the padlock probe of the present disclosure can be reverse complementary to about 1-2 bases, about 1-3 bases, about 1-5 bases, about 1-6 bases, about 1-7 bases, about 1-8 bases, about 1-9 bases, about 1-10 bases, about 1-12 bases, about 1-15 bases, about 1-20 bases, about 1-25 bases, about 1-30 bases, about 1-35 bases, about 1-40 bases, about 1-45 bases, or more of an miRNA. For example, the circularized probe can contain a sequence that is reverse complementary to miR-22-3p (e.g., the reverse complementary sequence can be 5′ ACAGTTCTTCAACTGGCAGCTT (SEQ ID NO: 92)) and a probe sequence (e.g., 5′ CCAGTGAATGCGAGTCCGACTAGGAGAGTAGGACAGCAGCCGT (SEQ ID NO: 81)). Thus, the circularized probe sequence that targets miR-22-3p can be 5′ ACAGTTCTTCAACTGGCAGCTT CCAGTGAATGCGAGTCCGACTAGGAGAGTAGGACAGCAGCCGT (circularized) (SEQ ID NO: 82). The circularized probe (or the portion of the circularized probe that comprises a probe sequence) or the padlock probe can comprise a sequence that is at least about 80%, at least about 82%, at least about 85%, at least about 87%, at least about 90%, at least about 92%, at least about 95%, at least about 97%, at least about 99%, or about 100% identical to a known probe sequence, to an universal probe, to a probe sequence that is commercially available, to a probe sequence that can be used with the methods of the present disclosure, or to the probe sequence 5′ CCAGTGAATGCGAGTCCGACTAGGAGAGTAGGACAGCAGCCGT (SEQ ID NO: 81). The circularized probe (or the portion of the circularized probe that is complementary or reverse complementary to an miRNA) or the padlock probe can comprise a sequence that is complementary or reverse complementary to at least about 80%, at least about 82%, at least about 85%, at least about 87%, at least about 90%, at least about 92%, at least about 95%, at least about 97%, at least about 99%, or about 100% of an miRNA sequence associated with PH (e.g., an miRNA associated with muscle function, an miRNA associated with blood oxygen content, an miRNA associated with erythrocyte function, an miRNA associated with hypoxia, and/or an miRNA that is associated with or regulates (or is linked to) BMPR2, ALK1, SMAD9, caveolin 1, and/or KCNK3 genes). For example, a circularized probe or the padlock probe can be complementary or reverse complementary to at least about 80%, at least about 85%, at least about 90%, at least about 95%, at least about 97%, at least about 99%, or about 100% of any one of the sequences shown in TABLE 1. An miRNA can have a sequence that is at least about 80%, at least about 82%, at least about 85%, at least about 87%, at least about 90%, at least about 92%, at least about 95%, at least about 97%, at least about 99%, or about 100% identical to any one of the sequences shown in TABLE 1. A circularized probe of the present disclosure that is reverse complementary to an miRNA (e.g., an miRNA associated with PH) can comprise, for example, a probe sequence (e.g., 5′ CCAGTGAATGCGAGTCCGACTAGGAGAGTAGGACAGCAGCCGT (SEQ ID NO: 81)) and a sequence as shown in TABLE 2. The circularized probe or the padlock probe can comprise at least about 50%, at least about 55%, at least about 60%, at least about 65%, at least about 70%, at least about 75%, at least 80%, at least 85%, at least 90%, at least 95%, at least 97%, at least 99%, or 100% of any one of the sequences in TABLE 2.

TABLE 2
List of circularized probe sequences comprising a sequence that is reverse
complementary to specific miRNAs

| Circularized probe | | |
|---|---|---|
| comprising a sequence | | |
| reverse complementary to | Reverse Complementary Sequence | SEQ ID NO: |
| miR-1-3p | 5′-ATACATACTTCTTTACATTCCA | 83 |
| miR-7-5p | 5′-ACAACAAAATCACTAGTCTTCCA | 84 |
| miR-16-5p | 5′-CGCCAATATTTACGTGCTGCTA | 85 |
| miR-17-5p | 5′-CTACCTGCACTGTAAGCACTTTG | 86 |
| miR-19a-3p | 5′-TCAGTTTTGCATAGATTTGCACA | 87 |
| miR-19b-3p | 5′-TCAGTTTTGCATGGATTTGCACA | 88 |
| miR-20a-5p | 5′-CTACCTGCACTATAAGCACTTTA | 89 |
| miR-20b-5p | 5′-CTACCTGCACTATGAGCACTTTG | 90 |
| miR-21-5p | 5′-TCAACATCAGTCTGATAAGCTA | 91 |
| miR-22-3p | 5′-ACAGTTCTTCAACTGGCAGCTT | 92 |
| miR-26a-5p | 5′-AGCCTATCCTGGATTACTTGAA | 93 |
| miR-26b-5p | 5′-ACCTATCCTGAATTACTTGAA | 94 |
| miR-27a-3p | 5′-GCGGAACTTAGCCACTGTGAA | 95 |
| miR-27b-3p | 5′-GCAGAACTTAGCCACTGTGAA | 96 |
| miR-29a-3p | 5′-TAACCGATTTCAGATGGTGCTA | 97 |
| miR-32-5p | 5′-TGCAACTTAGTAATGTGCAATA | 98 |
| miR-92a-3p | 5′-ACAGGCCGGGACAAGTGCAATA | 99 |
| miR-92b-3p | 5′-GGAGGCCGGGACGAGTGCAATA | 101 |
| miR-93-3p | 5′-CGGGAAGTGCTAGCTCAGCAGT | 102 |
| miR-93-5p | 5′-CTACCTGCACGAACAGCACTTTG | 103 |
| miR-100-3p | 5′-CATACCTATAGATACAAGCTTG | 104 |
| miR-100-5p | 5′-CACAAGTTCGGATCTACGGGTT | 105 |
| miR-103a-2-5p | 5′-CAAGGCAGCACTGTAAAGAAGCT | 106 |
| miR-106b-5p | 5′-ATCTGCACTGTCAGCACTTTA | 107 |
| miR-125b-1-3p | 5′-AGCTCCCAAGAGCCTAACCCGT | 108 |
| miR-128-3p | 5′-AAAGAGACCGGTTCACTGTGA | 109 |
| miR-129-5p | 5′-GCAAGCCCAGACCGCAAAAAG | 110 |
| miR-130a-3p | 5′-ATGCCCTTTTAACATTGCACTG | 111 |
| miR-133b | 5′-TAGCTGGTTGAAGGGGACCAAA | 112 |
| miR-135a-5p | 5′-TCACATAGGAATAAAAAGCCATA | 113 |
| miR-135b-5p | 5′-TCACATAGGAATGAAAAGCCATA | 114 |
| miR-143-3p | 5′-GAGCTACAGTGCTTCATCTCA | 115 |
| miR-143-5p | 5′-ACCAGAGATGCAGCACTGCACC | 116 |
| miR-144-3p | 5′-AGTACATCATCTATACTGTA | 117 |
| miR-146a-5p | 5′-AACCCATGGAATTCAGTTCTCA | 118 |
| miR-146b-3p | 5′-CCAGAACTGAGTCCACAGGGCA | 119 |
| miR-146b-5p | 5′-AGCCTATGGAATTCAGTTCTCA | 120 |
| miR-153-3p | 5′-GATCACTTTTGTGACTATGCAA | 121 |
| miR-181a-5p | 5′-ACTCACCGACAGCGTTGAATGTT | 122 |
| miR-181b-5p | 5′-ACCCACCGACAGCAATGAATGTT | 123 |
| miR-181c-5p | 5′-ACTCACCGACAGGTTGAATGTT | 124 |
| miR-181d-5p | 5′-ACCCACCGACAACAATGAATGTT | 125 |
| miR-186-5p | 5′-AGCCCAAAAGGAGAATTCTTTG | 126 |
| miR-192-5p | 5′-GGCTGTCAATTCATAGGTCAG | 127 |
| miR-199a-5p | 5′-GAACAGGTAGTCTGAACACTGGG | 128 |
| miR-204-5p | 5′-AGGCATAGGATGACAAAGGGAA | 129 |
| miR-206 | 5′-CCACACACTTCCTTACATTCCA | 130 |
| miR-210-3p | 5′-TCAGCCGCTGTCACACGCACAG | 131 |
| miR-214-3p | 5′-ACTGCCTGTCTGTGCCTGCTGT | 132 |
| miR-215-5p | 5′-GTCTGTCAATTCATAGGTCAT | 133 |
| miR-221-3p | 5′-GAAACCCAGCAGACAATGTAGCT | 134 |
| miR-222-3p | 5′-ACCCAGTAGCCAGATGTAGCT | 135 |
| miR-302b-5p | 5′-GAAAGCACTTCCATGTTAAAGT | 136 |
| miR-302c-3p | 5′-CCACTGAAACATGGAAGCACTTA | 137 |
| miR-302d-5p | 5′-GCAAGTGCCTCCATGTTAAAGT | 138 |
| miR-375-3p | 5′-TCACGCGAGCCGAACGAACAAA | 139 |
| miR-378a-3p | 5′-CCTTCTGACTCCAAGTCCAGT | 140 |
| miR-424-3p | 5′-ATAGCAGCGCCTCACGTTTTG | 141 |
| miR-424-5p | 5′-TTCAAAACATGAATTGCTGCTG | 142 |
| miR-451a | 5′-AACTCAGTAATGGTAACGGTTT | 143 |
| miR-452-5p | 5′-TCAGTTTCCTCTGCAAACAGTT | 144 |
| miR-486-5p | 5′-CTCGGGGCAGCTCAGTACAGGA | 145 |
| miR-490-5p | 5′-ACCCACCTGGAGATCCATGG | 146 |
| miR-494-3p | 5′-GAGGTTTCCCGTGTATGTTTCA | 147 |
| miR-503-3p | 5′-CCTGGCAGCGGAAACAATACCCC | 148 |
| miR-503-5p | 5′-CTGCAGAACTGTTCCCGCTGCTA | 149 |
| miR-519d-3p | 5′-CACTCTAAAGGGAGGCACTTTG | 150 |
| miR-548c-3p | 5′-GCAAAAGTAATTGAGATTTTTG | 151 |
| miR-550b-3p | 5′-CAGTGCCTGAGGGAGTAAGA | 152 |
| miR-892c-3p | 5′-TCCACTCAGAAAGGAAACAGTG | 153 |
| miR-1304-3p | 5′-GGGGTTCGAGGCTACAGTGAGA | 154 |
| miR-3615 | 5′-GAGCCGCGAGGAGCCGAGAGA | 155 |
| miR-4676-3p | 5′-AAGAGCCAGTGGTGAAACAGTG | 156 |
| miR-4693-5p | 5′-TGTGACAGTGAAATTCACAGTAT | 157 |
| miR-4716-5p | 5′-AGAAGGGGGAAGGAAACATGGA | 158 |
| miR-4772-3p | 5′-TCTGATCAGGCAAAGTTGCAGG | 159 |
| miR-6736-3p | 5′-CTGTGGGTAGAGAGGAGCTGA | 160 |
| miR-6787-3p, | 5′-CTGGAGAGGGCAGCAGCTGAGA | 161 |
| miR-6800-3p | 5′-GGGGCGATGCCAGGAGAGGTG | 162 |
| miR-6890-3p | 5′-CTGTGGGGCATAGGCAGTGG | 163 |

In some embodiments, the one or more circularized probe(s) is a random circularized probe or is a mixture of random circularized probes. In some embodiments, the circularized probe comprises a mixture of circularized probes. In some embodiments, the mixture comprises a mixture of circularized probes that are specific to various miRNAs. The rolling circle amplification using a circularized probe can include an annealing step and an amplification step. The rolling circle amplification using a circularized probe can include one step for the annealing and the amplification (e.g., the annealing and the amplification are performed concurrently). In some embodiments, the circularized probe or the padlock probe contains nucleotide substitutions. The circularized probe or the padlock probe can contain about one, about two, about 3, about 4, about 5, about 6, about 7, about 8, about 9, about 10, or more nucleotide substitutions (e.g., substitutions compared to a common/known or universal probe). The padlock probe can comprise a sequence that is at least about 80%, at least about 82%, at least about 85%, at least about 87%, at least about 90%, at least about 92%, at least about 95%, at least about 97%, at least about 99%, or about 100% identical to a known or universal probe sequence, a probe sequence that is commercially available, or a probe sequence that can be used with the methods of the present disclosure. In some embodiments, the probe (e.g., circularized probe or padlock probe) of the present disclosure has little complementarity with a human sequence, a mouse sequence, or any other NCBI library sequence, for example at most about 1%, at most about 5%, at most about 10%, at most about 12%, at most about 15%, at most about 20%, at most about 25%, at most about 30%, at most about 35%, at most about 40%, at most about 45%, at most about 50%, at most about 55%, at most about 60%, at most about 65%, at most about 70%, at most about 75%, at most about 80%, at most about 85%, at most about 90%, or at most about 95% sequence complementarity. Various methods and software programs can be used to determine the homology, identity, or complementarity between two or more peptides or nucleic acids, such as NCBI BLAST, Clustal W, MAFFT, Clustal Omega, AlignMe, Praline, or another suitable method or algorithm.

In some embodiments, one or more padlock probes are used. In some embodiments, the one or more padlock probes are random or is a mixture of random padlock probes. In some embodiments, the padlock probe is specific to an miRNA. In some embodiments, the mixture of padlock probes is specific to multiple miRNAs. In some embodiments, the mixture of random padlock probes targets random miRNAs. In some embodiments, the mixture comprises a mixture of padlock probes containing padlock probes that are specific to various miRNAs. In some embodiments, the rolling circle amplification contains both a circularized probe and a padlock probe. In some embodiments, the rolling circle amplification contains a mixture of circularized probes and padlock probes. Padlock probes are linear DNA probes where the terminal sequences can be designed to hybridize to two adjacent target sequences. The terminal sequences of the padlock probe can be designed to hybridize to different sequences within the same miRNA. For example, a terminal sequence of the padlock probe can be designed to hybridize to the 5′ end of an miRNA and the other terminal sequence of the padlock probe can be designed to hybridize to the 3′ end of the miRNA. DNA ligase can then ligate the termini of the padlock probe on a perfectly matching RNA template, thereby accurately distinguishing matched and mismatched substrates. The miRNA that is used as a template can subsequently be used as primer for rolling circle amplification, thereby linearly amplifying the target sequence. In some embodiments, a DNA ligase (e.g., T7 ligase or SplintR ligase) can ligate the termini of the padlock probe, for example, on a perfectly matching RNA template, thereby accurately distinguishing matched and mismatched substrates. SplintR Ligase, also known as PBCV-1 DNA Ligase or Chlorella virus DNA Ligase, can catalyze the ligation of adjacent, single-stranded DNA splinted by a complementary RNA strand. In some embodiments, an miRNA is used as a template. The miRNA that is used as a template can subsequently be used as a primer for rolling circle amplification, thereby linearly amplifying the target sequence.

In some embodiments, the nucleic acids can be labeled with a detectable label. In some embodiments, the nucleic acids are labeled with one or more detectable label. Nucleic acids can be labeled with tags that facilitate detection or purification. A variety of enzymatic or chemical methods can be used to generate nucleic acids labeled with radioactive phosphates, fluorophores, or nucleotides modified with biotin or digoxygenin for example. Nucleic acids can be labeled at the 5′ end, the 3′ end, or throughout the molecule depending on the application. In some embodiments, high specific activity probes are generated with label distributed throughout the nucleic acid, such as by, for example, nick translation, random priming, by PCR or in vitro transcription using labeled dNTPs or NTPs. Single-stranded or double-stranded DNA or RNA can be labeled at either ends of the molecule or randomly throughout the nucleic acid. Non-limiting examples of label include γ-³²P rATP, α-³²P dNTP, Biotin-dNTP, Fl-dNTP, and Fl terminator nucleotide.

In some embodiments, a labeled nucleic acid sequence is an oligonucleotide (or a probe). In some embodiments, the oligonucleotide is labeled with a detectable label. In some embodiments, the oligonucleotide is labeled with one or more detectable label(s). In some embodiments, amplification of nucleic acids is performed using rolling circle amplification. In some embodiments, a padlock probe is used in the rolling circle amplification reaction. In some embodiments, a circularized probe is used in the rolling circle amplification reaction. In some embodiments, the circularized probe is labeled with one or more detectable label(s). In some embodiments, the one or more padlock probes are labeled with a detectable label. In some embodiments, the padlock probe or the circularized probe comprises a phosphate at the 5′ end. In some embodiments, the padlock probe or the circularized probe is labeled with a radioactive label (e.g., ³²P). In some embodiments, the padlock probe or the circularized probe is labeled with a non-radioactive label. In some embodiments, the padlock probe or the circularized probe is labeled with a fluorescent label.

Exemplery detectable labels include fluorescent labels, bioluminescent labels, chemiluminescent labels, isotopic labels, nanoparticles, and metals. In some embodiments, the detectable label is a fluorescent label. Such labels can be detected, for example, by performing fluorescence imaging. In some embodiments, multiple cycles of fluorescence imaging are performed to allow detection of subsets of the target nucleic acids sequentially. A detectable label can refer to a molecule or substance capable of detection, including, but not limited to, fluorescers, chemiluminescers, chromophores, bioluminescent proteins, enzymes, enzyme substrates, enzyme cofactors, enzyme inhibitors, isotopic labels, semiconductor nanoparticles, dyes, metal ions, metal sols, ligands (e.g., biotin, streptavidin or haptens) and the like. A fluorescer can exhibit fluorescence in the detectable range. Particular examples of labels useful in the present disclosure include, but are not limited to, SYBR green, SYBR gold, a CAL Fluor dye such as CAL Fluor Gold 540, CAL Fluor Orange 560, CAL Fluor Red 590, CAL Fluor Red 610, and CAL Fluor Red 635, a Quasar dye such as Quasar 570, Quasar 670, and Quasar 705, an Alexa Fluor such as Alexa Fluor 350, Alexa Fluor 488, Alexa Fluor 546, Alexa Fluor 555, Alexa Fluor 594, Alexa Fluor 647, and Alexa Fluor 784, a cyanine dye such as Cy 3, Cy3.5, Cy5, Cy5.5, and Cy7, fluorescein, 2′, 4′, 5′, 7′-tetrachloro-4-7-dichlorofluorescein (TET), carboxyfluorescein (FAM), 6-carboxy-4′,5′-dichloro-2′,7′-dimethoxyfluorescein (JOE), hexachlorofluorescein (HEX), rhodamine, carboxy-X-rhodamine (ROX), tetramethyl rhodamine (TAMRA), FITC, dansyl, umbelliferone, dimethyl acridinium ester (DMAE), Texas red, luminol, and quantum dots, enzymes such as alkaline phosphatase (AP), beta-lactamase, chloramphenicol acetyltransferase (CAT), adenosine deaminase (ADA), aminoglycoside phosphotransferase (neo^{r}, G418¹) dihydrofolate reductase (DFIFR), hygromycin-B-phosphotransferase (FIPH), thymidine kinase (TK), β-galactosidase (lacZ), and xanthine guanine phosphoribosyltransferase (XGPRT), beta-glucuronidase (gus), placental alkaline phosphatase (PLAP), and secreted embryonic alkaline phosphatase (SEAP). Enzyme tags are used with their cognate substrate. A detectable label can also include chemiluminescent labels such as luminol, isoluminol, acridinium esters, and peroxyoxalate and bioluminescent proteins such as firefly luciferase, bacterial luciferase, Renilla luciferase, and aequorin. A detectable label can also include isotopic labels, including radioactive and non-radioactive isotopes, such as, ³H, ²H, ¹²⁰I, ¹²³I, ¹²⁴I, ¹²⁵I, ¹³¹I, ³⁵S, ^{U}C, ¹³C, ¹⁴C, ³²P, ¹⁵N, ¹³N, ¹¹⁰In, ^{U1}ln, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹⁵⁴Gd, ¹⁵⁵Gd, ¹⁵⁶Gd, ¹⁵⁷Gd, ¹⁵⁸Gd, ¹⁵O, ¹⁸⁶Re, ¹⁸⁸Re, ⁵¹M, ^{52m}Mn, ⁵⁵Co, ⁷²As, ⁷⁵Br, ⁷⁶Br, ^{82m}Rb, and ⁸³Sr. Detectable labels can also include color-coded microspheres of known fluorescent light intensities (e.g., microspheres with xMAP technology produced by Luminex (Austin, Tex.); microspheres containing quantum dot nanocrystals, for example, containing different ratios and combinations of quantum dot colors (e.g., Qdot nanocrystals produced by Life Technologies (Carlsbad, Calif.)), glass coated metal nanoparticles (e.g., SERS nanotags produced by Nanoplex Technologies, Inc. (Mountain View, Calif.)), barcode materials (e.g., sub-micron sized striped metallic rods such as Nanobarcodes produced by Nanoplex Technologies, Inc.), encoded microparticles with colored bar codes (e.g., CellCard produced by Vitra Bioscience, vitrabio.com), glass microparticles with digital holographic code images (e.g., CyVera microbeads produced by Illumina (San Diego, Calif.)), near infrared (MR) probes, and nanoshells. Detectable labels can also include contrast agents such as ultrasound contrast agents (e.g. SonoVue microbubbles comprising sulfur hexafluoride, Optison microbubbles comprising an albumin shell and octafluoropropane gas core, Levovist microbubbles comprising a lipid/galactose shell and an air core, Perflexane lipid microspheres comprising perfluorocarbon microbubbles, and Perflutren lipid microspheres comprising octafluoropropane encapsulated in an outer lipid shell), magnetic resonance imaging (MRI) contrast agents (e.g., gadodiamide, gadobenic acid, gadopentetic acid, gadoteridol, gadofosveset, gadoversetamide, gadoxetic acid), and radiocontrast agents, such as for computed tomography (CT), radiography, or fluoroscopy (e.g., diatrizoic acid, metrizoic acid, iodamide, iotalamic acid, ioxitalamic acid, ioglicic acid, acetrizoic acid, iocarmic acid, methiodal, diodone, metrizamide, iohexol, ioxaglic acid, iopamidol, iopromide, iotrolan, ioversol, iopentol, iodixanol, iomeprol, iobitridol, ioxilan, iodoxamic acid, iotroxic acid, ioglycamic acid, adipiodone, iobenzamic acid, iopanoic acid, iocetamic acid, sodium iopodate, tyropanoic acid, and calcium iopodate).

RNA molecules (e.g., miRNAs), or probes (e.g., a padlock probe or a circularized probe) can be primed, such as for amplification, by one or more oligonucleotide primers. In some embodiments, the oligonucleotide primer is a nucleic acid such as a DNA molecule or an RNA molecule. In some embodiments, oligonucleotide primers can comprise modifications. For example, potential modifications include 2′ fluoro nucleosides, LNA (locked nucleic acid), ZNA (zip nucleic acids), and PNA (Peptide Nucleic Acid). A primer can be at least about 6 bases in length. A primer can be at least about 8 bases in length. A primer can be about 6, about 7, or about 8 bases in length. A primer can be from about 6 bases up to about 10, from about 6 bases up to about 20, from about 6 bases up to about 30, from about 6 bases up to about 40, from about 6 bases up to about 50, or from about 6 bases up to about 100 bases in length. Priming of an RNA molecule or a probe can be done with one or more sequence and/or gene specific primers or with random primers. In some embodiments, the primer is gene specific. In some embodiments, the primer is a non-random primer. In some embodiments, the primer is specific to an miRNA. In some embodiments, the primer is specific to multiple miRNAs. In some embodiments, the primer is specific to an miRNA and probe sequence (e.g., circularized sequence). In some embodiments, the primer is specific to a circularized sequence. In some embodiments, the circularized sequence contains a probe sequence. In some embodiments, the circularized sequence contains an miRNA sequence. In some embodiments, the circularized sequence contains both an miRNA specific sequence and a probe sequence. In some embodiments, the circularized sequence is at least partially complementary to an miRNA sequence. In some embodiments, the circularized sequence contains a sequence that is complementary to an miRNA sequence. In some embodiments, the circularized sequence is at least partially reverse complementary to an miRNA sequence. In some embodiments, the circularized sequence contains a sequence that is reverse complementary to an miRNA sequence. In some cases, the primer is specific to a probe (e.g., a padlock probe or a circularized probe). In some cases, the primer is complementary to a probe and a particular miRNA sequence. In some cases, the primer is specific to a probe and a particular miRNA sequence. In some cases, the primer amplifies the probe across the miRNA target.

In some cases, the nucleic acid amplification is performed at ambient temperature (e.g., 30° C.). In some embodiments, the rolling circle amplification is performed at about room temperature, at about 0° C., at about 4° C., at about 10° C., at about 15° C., at about 20° C., at about 24° C., at about 25° C., at about 30° C., at about 35° C., at about 40° C., at about 45° C., at about 50° C., at about 55° C., at about 60° C., at about 65° C., at about 70° C., at about 75° C., at about 80° C., at about 85° C., at about 90° C., at about 95° C., at about 100° C., or more. In some embodiments, the rolling circle amplification is performed at least about room temperature, at least about 0° C., at least about 4° C., at least about 10° C., at least about 15° C., at least about 20° C., at least about 24° C., at least about 25° C., at least about 30° C., at least about 35° C., at least about 40° C., at least about 45° C., at least about 50° C., at least about 55° C., at least about 60° C., at least about 65° C., at least about 70° C., at least about 75° C., at least about 80° C., at least about 85° C., at least about 90° C., at least about 95° C., or at least about 100° C. In some embodiments, the rolling circle amplification is performed at most about room temperature, at most about 0° C., at most about 4° C., at most about 10° C., at most about 15° C., at most about 20° C., at most about 24° C., at most about 25° C., at most about 30° C., at most about 35° C., at most about 40° C., at most about 45° C., at most about 50° C., at most about 55° C., at most about 60° C., at most about 65° C., at most about 70° C., at most about 75° C., at most about 80° C., at most about 85° C., at most about 90° C., at most about 95° C., or at most about 100° C. In some embodiments, the rolling circle amplification is performed at about 30° C. In some embodiments, the rolling circle amplification is performed from about room temperature to about 30° C. In some embodiments, the rolling circle amplification is performed from about 20° C. to about 40° C. In some embodiments, the rolling circle amplification is performed from about 24° C. to about 30° C. In some embodiments, the rolling circle amplification is performed from about 15° C. to about 35° C. In some embodiments, the rolling circle amplification is performed from about 20° C. to about 35° C. In some embodiments, the rolling circle amplification is performed from about 24° C. to about 30° C. In some embodiments, the rolling circle amplification is performed at about 24° C. In some embodiments, the rolling circle amplification is performed at about room temperature. In some embodiments, the rolling circle amplification is performed for about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 35 hours, about 38 hours, about 40 hours, about 44 hours, about 48 hours, or more. In some embodiments, the rolling circle amplification is performed for at most about 30 minutes, at most about 1 hour, at most about 2 hours, at most about 3 hours, at most about 4 hours, at most about 5 hours, at most about 6 hours, at most about 7 hours, at most about 8 hours, at most about 9 hours, at most about 10 hours, at most about 11 hours, at most about 12 hours, at most about 13 hours, at most about 14 hours, at most about 15 hours, at most about 16 hours, at most about 17 hours, at most about 18 hours, at most about 19 hours, at most about 20 hours, at most about 21 hours, at most about 22 hours, at most about 23 hours, at most about 24 hours, at most about 25 hours, at most about 26 hours, at most about 27 hours, at most about 28 hours, at most about 29 hours, at most about 30 hours, at most about 35 hours, at most about 38 hours, at most about 40 hours, at most about 44 hours, at most about 48 hours, or more. In some embodiments, the rolling circle amplification is performed for at least about 30 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, at least about 24 hours, at least about 25 hours, at least about 26 hours, at least about 27 hours, at least about 28 hours, at least about 29 hours, at least about 30 hours, at least about 35 hours, at least about 38 hours, at least about 40 hours, at least about 44 hours, or at least about 48 hours. In some embodiments, the rolling circle amplification is performed for less than 2 hours. In some embodiments, the rolling circle amplification is performed from about 1 minute to about 1 hour, from about 1 minute to about 2 hours, from about 1 minute to about 3 hours, from about 1 minute to about 4 hours, from about 1 minute to about 5 hours, from about 1 minute to about 6 hours, from about 1 minute to about 7 hours, from about 1 minute to about 8 hours, from about 1 minute to about 10 hours, from about 1 minute to about 12 hours, from about 1 minute to about 15 hours, from about 1 minute to about 20 hours, from about 1 minute to about 24 hours, from about 1 minute to about 30 hours, from about 1 minute to about 38 hours, or from about 1 minute to about 48 hours. In some embodiments, the rolling circle amplification is performed from about 1 hour to about overnight. In some embodiments, the rolling circle amplification is performed from about 1 hour to about 24 hours. In some embodiments, the rolling circle amplification is performed from about 1 hour to about 12 hours. In some embodiments, the rolling circle amplification is performed from about 1 hour to about 16 hours. In some embodiments, the rolling circle amplification is performed from about 1 hour to about 8 hours. In some embodiments, the rolling circle amplification is performed from about 1 hour to about 2 hours. In some embodiments, the rolling circle amplification is performed from about 30 minutes to about 2 hours. In some embodiments, the rolling circle amplification is performed for less than about 2 hours and at room temperature. In some embodiments, the rolling circle amplification is performed for less than about 2 hours and at about 30° C. In some embodiments, the rolling circle amplification is performed for less than about 2 hours and at about 24° C. In some embodiments, RNA isolation is not performed prior to rolling circle amplification. In some embodiments, RNA isolation is not required for rolling circle amplification. In some embodiments, cDNA synthesis is not performed prior to rolling circle amplification. In some embodiments, cDNA synthesis is not required for rolling circle amplification. In some embodiments, rolling circle amplification includes target miRNA, detection template, ligase, and DNA polymerase. In some embodiments, the methods of the present disclosure can be used in lieu of a cDNA conversion step and/or the pre-amplification step, by for example, performing rolling circle amplification from an RNA/miRNA and a probe, for less than 2 hours and at room temperature or up to 30° C. In some embodiments, the methods of the present disclosure can be used in lieu of a cDNA conversion step and a quantification step, by for example, performing rolling circle amplification from an RNA/miRNA and a probe, for 2 hours or more and at room temperature or up to 30° C. In some embodiments, room temperature can be from about 20° C. to about 25° C.

A variety of kits and protocols to determine an expression profile by rolling circle amplification, polymerase chain reaction such as RT-PCR or real time quantitative PCR (RT-qPCR) can be used in the present disclosure. For example, reverse transcription of miRNAs can be performed using the TaqMan MicroRNA Reverse Transcription Kit (Applied Biosystems) according to manufacturer's recommendations. Briefly, miRNA can be combined with dNTPs, reverse transcriptase, and primers specific for a target miRNA. The resulting cDNA can be diluted and can be used for PCR reaction. The PCR can be performed according to the manufacturer's recommendation (Applied Biosystems). For example, cDNA can be combined with the TaqMan assay specific for the target miRNA and PCR reaction can be performed using 7900HT.

Determining a set of miRNAs can involve, for example, a microarray comprising miRNA-specific oligonucleotide probes. The microarray can include miRNA-specific oligonucleotide probes for the detection of miRNAs. Depending on the intended use of the microarray in the diagnosis or prognosis of a particular disease, probes for detecting different miRNAs can be included. A microarray for use in the diagnostic of pulmonary hypertension can include miRNA-specific oligonucleotide probes for one or more miRNAs (e.g., miRNAs associated with pulmonary hypertension, miRNAs associated with muscle function, blood oxygen content, oxygen tension, consumption, or utilization, erythrocyte function, hypoxia, or miRNAs associated with the regulation or expression of BMPR2, ACVRL1, SMAD9, caveolin 1, and/or KCNK3 genes).

The present disclosure provides for methods of assessing a clinical condition in a subject. The clinical condition can be pulmonary hypertension. Assessing a clinical condition in a subject can include, for example, extracting nucleic acids (e.g., miRNA, RNA, DNA, or cDNA) from a biological sample from a subject (e.g., a subject suspected of having pulmonary hypertension or at risk of developing pulmonary hypertension) and determining an expression profile of a set of an miRNA, or a biomarker, in the biological sample. The present disclosure also provides for methods of measuring an expression level of at least two miRNAs in a biological sample. Non-limiting examples of miRNAs that can be used for assessing a clinical condition include miR-16 (e.g., miR-16-5p), miR-451a, miR-486-5p, miR-92a, miR-221 (e.g., miR-221-3p), miR-144 (e.g., miR-144-3p), miR-222 (e.g., miR-222-3p), miR-1 (e.g., miR-1-3p), miR-133b, miR-206, miR-21 (e.g., miR-21-5p), miR-22 (e.g., miR-22-3p), miR-204, miR-181a (e.g., miR-181a-5p), miR-181b (e.g., miR-181b-5p), miR-181c-5p, miR-181d (e.g., miR-181d-5p), miR-214 (e.g., miR-214-3p), miR-146a (e.g., miR-146a-5p), miR-146b-3p, miR-146b-5p, miR-451a, miR-378 (e.g., miR-378a-3p), miR-29a (e.g., miR-29a-3p), miR-26a-5p, miR-21-5p, miR-22-3p, miR-135a, miR-186 (e.g., miR-186-5p), miR-210 (e.g., miR-210-3p), and any combination thereof. The miRNA can also be any miRNA associated with muscle function, blood oxygen content, erythrocyte function, hypoxia, and/or any miRNA associated with the regulation or expression of BMPR2, ALK1 (or ACVRL1), SMAD9, caveolin 1, and/or KCNK3 genes. An miRNA expression profile can be determined by, for example, amplifying (e.g., by PCR) nucleic acids (e.g., miRNA) from a biological sample from a subject. Amplified miRNAs can then be compared to a reference or to a plurality of miRNA expression profiles that are characteristic of different diseases. The subject can then be identified as having pulmonary hypertension based on such comparison. The subject can also be identified as having pulmonary hypertension if at least one miRNA in the subject's biological sample is significantly different than a reference or significantly different than the same miRNA in a control biological sample (e.g., biological sample from a healthy subject, or from other subjects with PH, or a biological sample taken from the same subject but at a different time point). In some embodiments, the at least one miRNA is at least two, at least three, at least four, at least five, at least six, at least seven, at least ten, or more miRNAs. In some embodiments, the at least one miRNA is a ratio (e.g., a ratio between at least one miRNA associated with muscle function compared to another miRNA associated with muscle function, or compared to at least one miRNA associated with blood oxygen content, or compared to at least one miRNA associated with the expression or regulation of BMPR2, ALK1 (or ACVRL1), SMAD9, caveolin 1, and/or KCNK3 genes). The ratio can also include comparing the levels of at least two miRNAs, at least three miRNAs, at least four miRNAs, at least five miRNAs, or more. The ratio can further include comparing the levels of a combination of miRNAs. The miRNA expression profile in a subject can also be used to determine the likelihood of disease or disease progression with respect to pulmonary hypertension. A subject can be diagnosed as having PH or at risk of developing PH when a downregulation or upregulation in at least one miRNA level is detected (e.g., downregulation or upregulation as compared to a reference). A reference can be a subject or a population of subjects having PH, or can be a healthy subject or a population of healthy subjects, can be a subject or a population of subjects without PH, or can be a subject or a population of subjects having increased blood pressure but no diagnosis of PH. The subject identified as having pulmonary hypertension can then undergo therapeutic intervention (e.g., oxygen therapy or exercise training).

The methods of the present disclosure can include comparing two samples or a plurality of samples (e.g., two biological samples or a biological sample and a reference). For example, one sample can be from a subject having PH or suspected of having PH, and the other sample can be a reference (e.g., a biological sample taken from the same subject at a different time point, or a biological sample from a healthy subject or a subject without PH, or the reference can be a predetermined value). In some embodiments, comparing the samples include comparing at least one miRNA level between the samples. A subject can be identified as having PH if, for example, an miRNA level in a sample is found to be significantly different when compared to the same miRNA level in another sample or a reference. Determining whether the difference is significant can be performed by using a linear regression analysis, Mann-Whitney U-test, Fisher's exact test, t-test, Analysis of Variance (ANOVA), chi-square test, Kolmogorov-Smirnov test, Wilcoxon signed rank test, or a combination thereof. In some embodiments, a difference is found to be significant by determining a p-value. In some embodiments, a p-value of less than or equal to 0.05 is considered significant.

The methods of the present disclosure can further include determining whether a subject is a responder to therapy. For example, a subject can be identified as a responder to therapy (e.g., exercise training or oxygen intake) if an miRNA level from a biological sample is determined to be upregulated or downregulated after therapy as compared to before therapy. A subject can also be identified as being a responder to therapy if a ratio of at least one miRNA is significantly different when the ratio is compared between before and after therapy or compared to a reference. For example, a subject can be identified as a responder to therapy if a ratio of a composite expression level of miR-22-3p and miR-21-5p relative to miR-451 and Spike RNA decreases or increases after therapy as compared to before therapy. In some cases, the same miRNA level is compared before and after therapy (e.g., miR-22-3p). The miRNA can be associated with muscle function, blood oxygen content (e.g., erythrocyte function or hypoxia), or with the regulation or expression of BMPR2, ALK1 (or ACVRL1), SMAD9, caveolin 1, and/or KCNK3 genes, and any combination thereof. In some cases, a heat map analysis of miRNAs is determined. In some cases, the heat map analysis can help determine whether a subject is responsive to pulmonary hypertension treatment. In some cases, the heat map analysis can help with the classification of the pulmonary hypertension of a subject (e.g., classification based on endotype, disease status, or WHO classification).

The present disclosure can also include kits for diagnosing a disease (e.g., pulmonary hypertension). The kit can be used for determining an expression profile of a predetermined set of an miRNA in a biological sample, such as in a blood, plasma and/or serum sample including whole blood, plasma, serum or fractions thereof, or in a sample comprising peripheral blood mononuclear cells, erythrocytes, leukocytes and/or thrombocytes. In some cases, one or more reference expression profiles are also provided to show the expression profile of the same set of miRNAs in the same type of biological sample obtained from one or more healthy subjects or from a reference. A comparison to a reference expression profile or to an expression profile from a healthy subject can aid in the diagnosis of the disease.

The kit can be a test kit for detecting miRNAs in a sample by nucleic acid hybridization and amplification such as rolling circle amplification, PCR or RT-PCR. The kit can include probes and/or primers for detecting a predetermined set of miRNAs. Further, the kit can include enzymes and reagents, e.g. for the cDNA synthesis from miRNAs prior to PCR (e.g., qRT-PCR). The kit of the present disclosure can include any one of the following: a DNA ligase (e.g., T7 ligase or SplintR ligase), a ligation mixture, a buffer, BSA, dNTPs, a DNA polymerase (e.g., phi29), a denaturing buffer, a freeze-dried cake, an RNA, an miRNA, a probe, a DNA, a primer (e.g., forward/reverse primer), hexamers, salts, an oligonucleotide, a padlock probe, and/or a circularized probe. The rolling circle amplification of the present disclosure can use a commercially available kit for the amplification reaction (e.g., Illustra™ Ready-To-Go™ GenomiPhi™ V3 DNA Amplification Kit from GE Healthcare). In some embodiments, the kit can be used to amplify a whole genome. The kit can contain a freeze-dried cake that contains DNA polymerase (e.g., phi29 polymerase), nucleotides, salts, buffers, and/or random hexamers (e.g., random hexamer primers). In some embodiments, the kit does not contain random hexamers. In some embodiments, the kit includes a probe (e.g., padlock probe and/or complimentary probe). In some embodiments, the kit includes an miRNA. In some embodiments, the kit includes a mixture of probes. In some embodiments, the kit includes a mixture of miRNAs.

The kit for diagnosing a disease can include a predetermined set of miRNAs and/or can include oligonucleotides that target one or more miRNAs for the diagnosis of pulmonary hypertension. A kit for diagnosing PH can include tools for determining the expression profile of one or more miRNAs from one or more patients. In some cases, the kit can include oligonucleotides that are complementary to at least one miRNA associated with PH. For example, the miRNA can be associated with muscle function, blood oxygen content, oxygen tension, consumption, or utilization, hypoxia, erythrocyte function, and/or can regulate the expression of BMPR2, ALK1 (or ACVRL1), SMAD9, caveolin 1, and/or KCNK3 genes. The miRNA can be any miRNA associated with PH or any miRNA used to determine PH in a subject. Non-limiting examples of miRNAs include miR-16 (e.g., miR-16-5p), miR-451a, miR-486-5p, miR-92a, miR-221 (e.g., miR-221-3p), miR-143-3p, miR-143-5p, miR-144 (e.g., miR-144-3p), miR-222 (e.g., miR-222-3p), miR-1 (e.g., miR-1-3p), miR-133b, miR-206, miR-21 (e.g., miR-21-5p), miR-22 (e.g., miR-22-3p), miR-204, miR-181a (e.g., miR-181a-5p), miR-181b (e.g., miR-181b-5p), miR-181c-5p, miR-181d (e.g., miR-181d-5p), miR-214 (e.g., miR-214-3p), miR-146a (e.g., miR-146a-5p), miR-146b-3p, miR-146b-5p, miR-424-3p, miR-424-5p, miR-451, miR-378 (e.g., miR-378a-3p), miR-29a (e.g., miR-29a-3p), miR-26a, miR-21-5p, miR-22-3p, miR-135a, miR-186 (e.g., miR-186-5p), miR-503-3p, miR-503-5p, miR-210 (e.g., miR-210-3p), and any combination thereof. The kit can include directions and/or reagents and/or oligonucleotides for determining at least 1, 2, 3, 4, 5, 6, 7, 10, 15, 20, 24, or more miRNAs.

In some cases, the kit includes oligonucleotides complementary to at least one miRNA associated with BMPR2 such as miR-20 (e.g., miR-20a-5p), miR-21-5p, miR-129-5p, miR-92a-3p, miR-19b-3p, miR-19a-3p, miR-17-5p, miR-100-5p, miR-302c-3p, miR-130a-3p, miR-181c-5p, miR-128-3p, miR-215-5p, miR-7-5p, miR-192-5p, miR-93-5p, miR-26b-5p, miR-93-3p, miR-135a-5p, miR-181a-5p, miR-106b-5p, miR-20b-5p, miR-519d-3p, miR-32-5p, miR-181b-5p, miR-181d-5p, miR-92b-3p, miR-27a-3p, miR-27b-3p, miR-548c-3p, miR-135b-5p, miR-4716-5p, miR-4772-3p, miR-3615, miR-1304-3p, miR-125b-1-3p, miR-6890-3p, miR-6800-3p, miR-6787-3p, miR-6736-3p, miR-4693-5p, miR-103a-2-5p, miR-302d-5p, miR-302b-5p, miR-550b-3p, miR-892c-3p, miR-4676-3p, miR-452-5p, miR-100-3p, miR-490-5p, miR-153-3p, miR-204, miR-135a, miR-375 (e.g., miR-375-3p), and/or miR-494 (e.g., miR-494-3p).

In some cases, the kit includes oligonucleotides complementary to at least one miRNA associated with caveolin 1 such as miR-34c-5p, miR-34b-5p, miR-124-3p, miR-103a-3p, miR-7-5p, miR-26b-5p, miR-199a-5p, miR-203a-3p, miR-107, ssc-miR-199a-5p, miR-192-5p, miR-17-5p, miR-20 (e.g., miR-20a-5p), miR-93-5p, miR-106a-5p, miR-194-5p, miR-106b-5p, miR-20b-5p, miR-526b-3p, miR-519d-3p, miR-3609, miR-548ah-5p, miR-4796-3p, miR-3973, miR-873-5p, miR-520h, miR-520g-3p, miR-4463, miR-1238-3p, miR-6749-3p, miR-6792-3p, miR-4691-5p, miR-627-3p, miR-660-3p, miR-5193, miR-670-3p, miR-4277, miR-584-3p, miR-5004-3p, miR-1261, miR-4791, miR-3201, miR-766-5p, miR-3140-3p, miR-4722-5p, miR-4468, miR-4673, miR-4645-5p, miR-4692, miR-4514, miR-4459, miR-556-5p, miR-208b-5p, miR-208a-5p, miR-6165, miR-6753-5p, miR-1911-3p, miR-338-5p, miR-4517, and/or mmu-miR-124-3p.

In some cases, the kit includes oligonucleotides complementary to at least one miRNA associated with SMAD9 such as miR-106b-5p, miR-203a-3p, miR-574-5p, miR-653-5p, miR-5585-3p, miR-190a-3p, miR-6867-5p, miR-223-5p, miR-511-3p, miR-5011-5p, miR-1277-5p, miR-665, miR-887-5p, miR-6780a-5p, miR-6779-5p, miR-3689c, miR-3689b-3p, miR-3689a-3p, miR-30b-3p, miR-1273h-5p, miR-6788-5p, miR-30c-2-3p, miR-30c-1-3p, miR-6799-5p, miR-6883-5p, miR-6785-5p, miR-4728-5p, miR-149-3p, miR-7106-5p, miR-7160-5p, miR-4722-5p, miR-6884-5p, miR-485-5p, miR-1827, miR-4649-3p, miR-4768-3p, miR-4478, miR-4419b, miR-3929, miR-940, miR-6893-5p, miR-6808-5p, miR-890, miR-34b-3p, and/or miR-606.

In some cases, the kit includes oligonucleotides complementary to at least one miRNA associated with KCNK3 such as miR-6788-5p, miR-30c-2-3p, miR-30c-1-3p, miR-6778-5p, miR-1233-5p, miR-6766-5p, miR-6756-5p, miR-608, miR-4651, miR-7110-5p, miR-6842-5p, miR-6752-5p, miR-6825-5p, miR-6785-5p, miR-6883-5p, miR-4728-5p, miR-8085, miR-149-3p, miR-6731-5p, miR-6878-5p, miR-4763-3p, miR-1207-5p, miR-6722-3p, miR-1909-3p, miR-4707-5p, miR-6732-5p, miR-4296, miR-4322, miR-4265, miR-4417, miR-6816-5p, miR-3196, miR-3180-3p, miR-3180, miR-3656, miR-3621, miR-423-5p, miR-3184-5p, miR-365b-5p, miR-365a-5p, miR-8052, miR-3199, miR-6778-3p, miR-150-5p, miR-6814-5p, and/or miR-3691-3p.

In some cases, the kit includes oligonucleotides complementary to at least one miRNA associated with ACVRL1 such as miR-6833-3p, miR-4768-5p, miR-6773-5p, miR-6724-5p, miR-6873-3p, miR-4684-5p, miR-296-5p, miR-942-5p, miR-6817-3p, miR-7110-3p, miR-5088-3p, miR-6756-3p, miR-3127-3p, miR-1237-5p, miR-128-1-5p, miR-128-2-5p, miR-4488, miR-4505, miR-4514, miR-4690-5p, miR-4692, miR-4697-5p, miR-4731-5p, miR-5787, miR-637, miR-6808-5p, miR-6846-5p, miR-6848-5p, miR-6877-5p, miR-6893-5p, miR-940, miR-1224-5p, miR-4751, miR-4753-5p, miR-5004-5p, and/or miR-7160-5p.

The methods of the present disclosure can also include preparing a report. For example, a report can indicate that a subject has pulmonary hypertension or is at risk of developing pulmonary hypertension based on an miRNA obtained from a subject's biological sample. The miRNA obtained from a subject can be compared to an miRNA obtained from a healthy subject or a subject who does not have PH. Alternatively, the miRNA obtained from a subject can be compared to an miRNA obtained from another subject with PH. The miRNA can also be compared to another comparable biological sample obtained at a different time point from the same subject.

In some cases, differences in the microRNA expression are measured from biological samples. In some cases, a differentially expressed microRNA can qualitatively have expression altered, including an activation or inactivation, in, e.g., normal versus diseased PH tissue, or in biological samples from a normal subject versus a diseased subject. A qualitatively regulated microRNA can exhibit an expression pattern within a PH sample or cell type that is detectable by standard techniques. Some microRNAs can be expressed in one PH sample or cell type, and not in another, or expressed at different levels between different cell types or different samples. Thus, the difference in expression can be quantitative, e.g., in that expression is modulated, up-regulated, resulting in an increased amount of microRNA, or down-regulated, resulting in a decreased amount of microRNA. The degree to which expression differs can be, for example, only large enough to quantify via standard characterization techniques such as expression arrays, next generation sequencing (NGS), quantitative reverse transcriptase PCR, northern blot analysis, real-time PCR, in situ hybridization and RNase protection.

The methods of the present disclosure can also include measuring the microRNAs for determining PH in a subject, or for identifying a subject at risk of developing PH, or for determining improvement in PH in a subject. Measuring RNA can be performed by high throughput sequencing. High throughput sequencing can involve sequencing-by-synthesis, sequencing-by-ligation, and ultra-deep sequencing. Sequence-by-synthesis can be initiated using sequencing primers complementary to the sequencing element on nucleic acid tags. The method involves detecting the identity of each nucleotide immediately after (substantially real-time) or upon (real-time) the incorporation of a labeled nucleotide or nucleotide analogue into a growing strand of a complementary nucleic acid sequence in a polymerase reaction. After incorporation of a label nucleotide, a signal can be measured. Examples of labels that can be used to label nucleotide or nucleotide analogs for sequencing-by-synthesis include, but are not limited to, chromophores, fluorescent moieties, enzymes, antigens, heavy metal, magnetic probes, dyes, phosphorescent groups, radioactive materials, chemiluminescent moieties, scattering or fluorescent nanoparticles, Raman signal generating moieties, and electrochemical detection moieties. Sequencing-by-synthesis can generate, for example, at least 1,000, at least 5,000, at least 10,000, at least 20,000, 30,000, at least 40,000, at least 50,000, at least 100,000 or at least 500,000 reads per hour. Such reads can have at least 40, at least 45, at least 50, at least 60, at least 70, at least 80, at least 90, at least 100, at least 120 or at least 150 bases per read.

Sequencing-by-synthesis can be performed on a solid surface (or a chip) using fold-back PCR and anchored primers. Since microRNAs occur as small nucleic acid fragments—adaptors can be added to the 5′ and 3′ ends of the fragments. Nucleic acid fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded nucleic acid molecules of the same template in each channel of the flow cell. Primers, polymerase and four fluorophore-labeled, reversibly terminating nucleotides are used to perform sequential sequencing. After nucleotide incorporation, a laser is used to excite the fluorophores, an image is captured and the identity of the first base is recorded. The 3′ terminators and fluorophores from each incorporated base are removed and the incorporation, detection and identification steps are repeated. This technology is used, for example, in the Illumina® sequencing platform.

Another sequencing method involves hybridizing the amplified regions to a primer complementary to the sequence element in an LST (a file listing the names of fasta files). This hybridization complex is incubated with a polymerase, ATP sulfurylase, luciferase, apyrase, and the substrates luciferin and adenosine 5′ phosphosulfate. Next, deoxynucleotide triphosphates corresponding to the bases A, C, G, and T (U) are added sequentially. Each base incorporation is accompanied by release of pyrophosphate, converted to ATP by sulfurylase, which drives synthesis of oxyluciferin and the release of visible light. Since pyrophosphate release is equimolar with the number of incorporated bases, the light given off is proportional to the number of nucleotides adding in any one step. The process is repeated until the entire sequence is determined. Yet another sequencing method involves a four-color sequencing by ligation scheme (degenerate ligation), which involves hybridizing an anchor primer to one of four positions. Then an enzymatic ligation reaction of the anchor primer to a population of degenerate nonamers that are labeled with fluorescent dyes is performed. At any given cycle, the population of nonamers that is used is structure such that the identity of one of its positions is correlated with the identity of the fluorophore attached to that nonamer. To the extent that the ligase discriminates for complementarily at that queried position, the fluorescent signal allows the inference of the identity of the base. After performing the ligation and four-color imaging, the anchor primer:nonamer complexes are stripped and a new cycle begins. In some cases, image sequence information is obtained after performing ligation. In some cases, high throughput sequencing involves the use of ultra-deep sequencing.

MicroRNAs can be sequenced using microRNA sequencing. MicroRNA sequencing (miRNA-seq) is a type of RNA Sequencing (RNA-Seq) which uses next-generation sequencing or massively parallel high-throughput DNA sequencing to sequence microRNAs. miRNA-seq differs from other forms of RNA-Seq in that input material is often enriched for small RNAs. miRNA-seq provides tissue specific expression patterns, corresponding to disease associations and microRNAs isoforms. miRNA-seq is also used for the discovery of previously-uncharacterized microRNAs.

### EXAMPLES

### Example 1: Identification of miRNAs

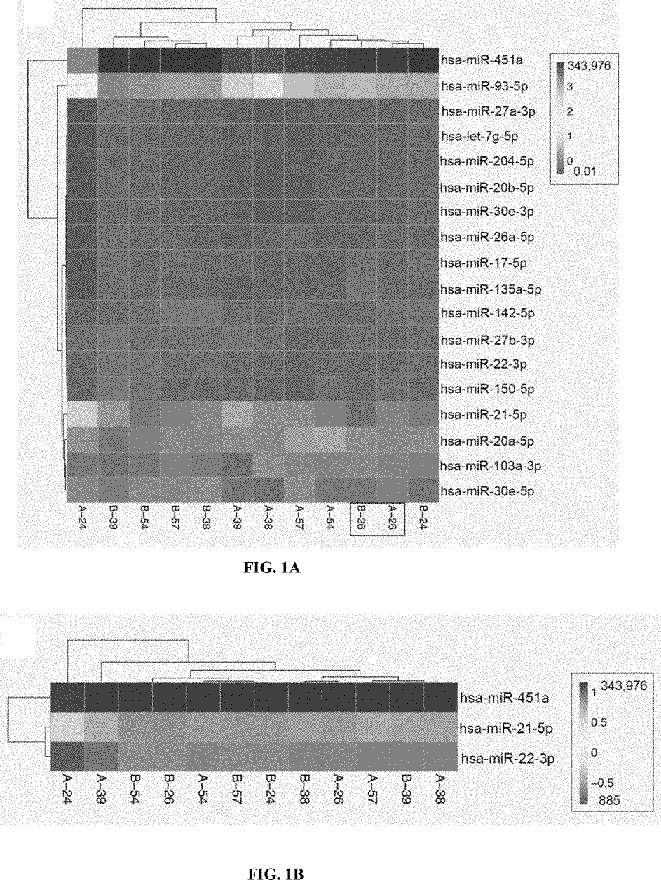

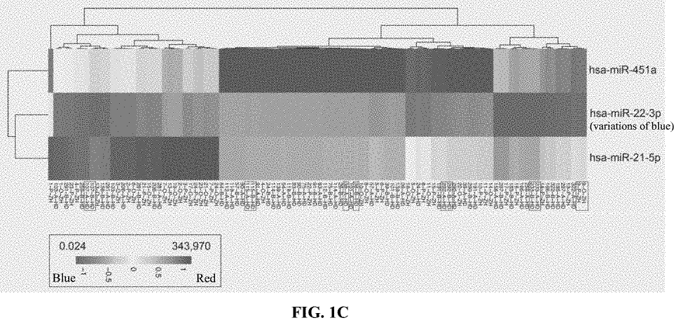

In this experiment, RNA from whole blood and serum samples (n=12 and n=6, respectively) from PH patients who received exercise training intervention was analyzed to identify potential miRNAs involved in PH. Eighteen (18) miRNAs were chosen based on RNA sequencing of whole blood RNA (data not shown). LNA-modified primers that target the identified 18 miRNAs were purchased: hsa-let-7g-5p, hsa-miR-17-5p, hsa-miR-20a-5p, hsa-miR-20b-5p, hsa-miR-21-5p, hsa-miR-22-3p, hsa-miR-26a-5p, hsa-miR-27a-3p, hsa-miR-27b-5p, hsa-miR-30e-3p, hsa-miR-30e-5p, hsa-miR-93-5p, hsa-miR-103a-3p, hsa-miR-135a-5p, hsa-miR-142-5p, hsa-miR-150-5p, hsa-miR-204-5p, and hsa-miR-451a. The LNA-modified primers were detected by quantitative PCR and calculated relative to synthetic Spike RNA. Hierarchical cluster analysis was used to identify miRNAs that showed changed levels when comparing samples taken before and after exercise training (FIG. 1A). The focus was evaluating the magnitude of the change, irrespective of the direction of the change (increased or decreased). Samples from a single donor were clustered together (represented by boxes) while the samples were clustered apart for the remainder of the donors (n=5) (FIG. 1A). This experiment identified three miRNAs: miR-22-3p, miR-451a, and miR-21-5p (FIG. 1B). None of the paired samples clustered together (FIG. 1B).

In addition, hierarchical cluster analysis was performed for a larger sample size of patients (patients subjected to exercise training or oxygen/placebo intake therapy) (FIG. 1C, TABLE 5). This hierarchical cluster analysis included 52 donors and 104 samples in total. The cluster analysis showed that miR-21-5p and miR-22-3p clustered together, separately from miR-451a (FIG. 1C). Additionally, the clustered miRNAs separated the before and after intervention measurements in 46 donors, and only 6 donor data clustered together (clustered data is represented by boxes; FIG. 1C).

MiRNA Expression: Real time PCR was performed in triplicate with 0.1 ng of cDNA per reaction using a 7900HT Fast Real-Time PCR instrument (Applied Biosystems/Life Technologies, Grand Island, N.Y., United States) in a 10 μL volume. The PCR reactions were run with LNA-modified primers and SYBR Green master mix (Exiqon, Denmark/now Qiagen) in 384-well plate under the following conditions: 95° C. for 10 min, followed by 45 cycles of 95° C. for 10 s and 60° C. for 1 min, followed by a hold at 4° C. The LNA-modified primers that target the miRNAs shown in TABLE 3 and Spike6 (target the synthetic spike RNA, UniSP6, supplied in the cDNA kit) were used. The results obtained for the 18 hsa-miRNAs relative to spike RNA are shown in TABLE 4 (also refer to FIG. 1A). The miRNA levels (e.g., hsa-miR-22-3p, hsa-miR-21-5p, and hsa-miR-451a) from samples obtained before or after exercise training or following placebo/oxygen intake therapy are shown in TABLE 5 (also refer to FIG. 1C). TABLE 4 and TABLE 5 show Ct values relative to spike RNA multiplied by 10000.

TABLE 3
LNA-modified primers that target miRNA

| miRNA | Sequence | SEQ ID NO: |
|---|---|---|
| hsa-let-7g-5p | 5′ UGAGGUAGUAGUUUGUACAGUU | 164 |
| hsa-miR-17-5p | 5′ CAAAGUGCUUACAGUGCAGGUAG | 4 |
| hsa-miR-20a-5p | 5′ UAAAGUGCUUAUAGUGCAGGUAG | 7 |
| hsa-miR-20b-5p | 5′ CAAAGUGCUCAUAGUGCAGGUAG | 8 |
| hsa-miR-21-5p | 5′ UAGCUUAUCAGACUGAUGUUGA | 9 |
| hsa-miR-22-3p | 5′ AAGCUGCCAGUUGAAGAACUGU | 10 |
| hsa-miR-26a-5p | 5′ UUCAAGUAAUCCAGGAUAGGCU | 11 |
| hsa-miR-27a-3p | 5′ UUCACAGUGGCUAAGUUCCGC | 13 |
| hsa-miR-27b-5p | 5′ AGAGCUUAGCUGAUUGGUGAAC | 165 |
| hsa-miR-30e-3p | 5′ CUUUCAGUCGGAUGUUUACAGC | 166 |
| hsa-miR-30e-5p | 5′ UGUAAACAUCCUUGACUGGAAG | 167 |
| hsa-miR-93-5p | 5′ CAAAGUGCUGUUCGUGCAGGUAG | 20 |
| hsa-miR-103a-3p | 5′ AGCAGCAUUGUACAGGGCUAUGA | 168 |
| hsa-miR-135a-5p | 5′ UAUGGCUUUUUAUUCCUAUGUGA | 30 |
| hsa-miR-142-5p | 5′ CAUAAAGUAGAAAGCACUACU | 169 |
| hsa-miR-150-5p | 5′ UCUCCCAACCCUUGUACCAGUG | 170 |
| hsa-miR-204-5p | 5′ UUCCCUUUGUCAUCCUAUGCCU | 46 |
| hsa-miR-451a | 5′ AAACCGUUACCAUUACUGAGUU | 60 |

TABLE 4
Serum miRNA levels determined relative to spike RNA from
samples obtained before or after exercise training

| Sample ID | miR-17-5p | let-7g-5p | miR-103a-3p | miR-20a-5p | miR-20b-5p | miR-21-5p |
|---|---|---|---|---|---|---|
| Before-57 | 1450.68 | 10.00 | 6028.19 | 9396.34 | 2.14 | 5855.24 |
| After-57 | 2585.42 | 2.15 | 16863.95 | 28285.11 | 0.90 | 20395.45 |
| Before-54 | 491.86 | 4.21 | 11185.35 | 15112.91 | 1.09 | 10001.12 |
| After-54 | 426.47 | 0.01 | 7569.24 | 17269.53 | 0.01 | 6371.90 |
| Before-39 | 465.07 | 2.15 | 1747.02 | 2113.98 | 0.01 | 8420.29 |
| After-39 | 2376.13 | 22.34 | 3588.93 | 13364.28 | 40.16 | 21958.66 |
| Before-38 | 1243.64 | 14.48 | 4344.74 | 6901.56 | 323.07 | 8125.19 |
| After-38 | 1778.50 | 437.19 | 10432.80 | 9390.54 | 0.67 | 10189.21 |
| Before-26 | 2604.85 | 8.26 | 12299.95 | 12683.78 | 2.03 | 3234.26 |
| After-26 | 882.41 | 12.59 | 11199.18 | 13863.31 | 19.67 | 10913.51 |
| Before-24 | 1881.65 | 10.58 | 12141.77 | 21107.39 | 0.33 | 10514.07 |
| After-24 | 250.07 | 33.04 | 4117.21 | 8168.28 | 14.18 | 17078.98 |

| Sample ID | miR-22-3p | miR-26a-5p | miR-27a-3p | miR-27b-3p | miR-135a-5p | miR-30e-5p |
|---|---|---|---|---|---|---|
| Before-57 | 1819.48 | 1020.16 | 15.09 | 2039.90 | 505.75 | 8234.08 |
| After-57 | 2284.77 | 3916.42 | 230.53 | 3505.18 | 2730.64 | 20971.34 |
| Before-54 | 5193.04 | 295.18 | 1708.29 | 2603.29 | 7.07 | 25983.71 |
| After-54 | 1255.95 | 79.76 | 6.71 | 1410.43 | 48.08 | 4703.51 |
| Before-39 | 1310.36 | 614.82 | 1264.88 | 1950.02 | 345.31 | 3016.37 |
| After-39 | 2513.11 | 88.25 | 12.84 | 5435.86 | 77.77 | 4772.99 |
| Before-38 | 1636.78 | 255.83 | 74.77 | 1784.62 | 515.34 | 9022.08 |
| After-38 | 2428.45 | 1746.88 | 56.29 | 3013.86 | 1707.11 | 3536.73 |
| Before-26 | 1703.77 | 157.27 | 8.57 | 3156.71 | 3427.43 | 5710.69 |
| After-26 | 884.53 | 1219.22 | 26.70 | 1871.35 | 1393.82 | 9792.23 |
| Before-24 | 2091.62 | 203.14 | 182.75 | 2482.96 | 118.53 | 5776.70 |
| After-24 | 2325.63 | 562.54 | 106.33 | 3089.54 | 472.87 | 6910.42 |

| Sample ID | miR-30e-3p | miR-93-5p | miR-142-5p | miR-150-5p | miR-204-5p | miR-451a |
|---|---|---|---|---|---|---|
| Before-57 | 4.66 | 15429.57 | 3850.84 | 1026.34 | 1.07 | 104635.84 |
| After-57 | 1.00 | 41794.08 | 5761.58 | 1198.11 | 702.70 | 167789.98 |
| Before-54 | 223.30 | 34497.77 | 1632.10 | 5405.73 | 5.09 | 343976.44 |
| After-54 | 39.87 | 19579.83 | 1277.68 | 2446.21 | 96.12 | 102612.51 |
| Before-39 | 0.39 | 5438.99 | 17.27 | 2046.95 | 4.11 | 75230.13 |
| After-39 | 4.84 | 34085.71 | 1648.58 | 1829.20 | 5.00 | 119988.88 |
| Before-38 | 0.47 | 11818.97 | 3942.44 | 673.03 | 2.14 | 87243.89 |
| After-38 | 0.98 | 29558.81 | 2609.45 | 1243.98 | 0.01 | 79633.92 |
| Before-26 | 1.77 | 26433.22 | 1761.53 | 1704.23 | 4.73 | 121960.56 |
| After-26 | 53.88 | 27891.37 | 2392.51 | 3082.02 | 0.01 | 155277.76 |
| Before-24 | 36.74 | 34899.28 | 3993.53 | 1315.21 | 0.41 | 223464.16 |
| After-24 | 16.06 | 26467.74 | 2001.91 | 1923.04 | 1.78 | 49594.24 |

TABLE 5
Serum/plasma miRNA levels determined relative to spike
RNA from samples obtained before or after exercise
training, or following placebo or oxygen intervention

| Sample ID | miR-22-3p | miR-21-5p | miR-451a |
|---|---|---|---|
| Before-24 | 2092.07 | 9148.45 | 223511.48 |
| Before-26 | 1703.83 | 10480.11 | 121964.95 |
| Before-38 | 1636.86 | 7395.93 | 87248.24 |
| Before-39 | 1310.05 | 8148.27 | 75212.30 |
| Before-54 | 5192.94 | 8218.50 | 343969.78 |
| Before-57 | 1819.70 | 4054.27 | 104648.71 |
| Before-101 | 91.94 | 1028.53 | 12090.14 |
| Before-112 | 178.98 | 1715.16 | 27730.60 |
| Before-114 | 134.23 | 787.57 | 20974.30 |
| Before-115 | 129.47 | 1107.68 | 37582.87 |
| Before-119 | 56.91 | 638.01 | 11680.34 |
| Before-139 | 106.90 | 1174.27 | 13155.82 |
| Before-75 | 14.19 | 175.88 | 2628.49 |
| Before-83 | 250.61 | 1885.50 | 33133.74 |
| Before-90 | 149.89 | 1285.25 | 29963.79 |
| Before-91 | 166.21 | 1371.40 | 26730.12 |
| Before-103 | 8.20 | 3659.23 | 1473.31 |
| Before-107 | 7.72 | 4371.57 | 2944.24 |
| Before-158 | 1.77 | 1068.78 | 685.24 |
| Before-166 | 11.15 | 2101.81 | 2805.21 |
| Before-185 | 8.56 | 2325.36 | 2416.00 |
| Before-191 | 26.27 | 3604.02 | 10768.10 |
| Before-206 | 1.17 | 889.65 | 299.02 |
| Before-233 | 5.70 | 1842.52 | 1826.42 |
| Before-259 | 28.20 | 7744.50 | 4399.83 |
| Before-281 | 5.87 | 3620.49 | 1129.64 |
| Before-290 | 33.89 | 5939.12 | 25225.78 |
| Before-291 | 25.71 | 6542.03 | 3272.73 |
| Before-292 | 13.87 | 1846.62 | 6990.71 |
| Before-297 | 18.69 | 6355.00 | 5556.67 |
| Before-322 | 51.77 | 14441.36 | 18563.34 |
| After-24 | 2326.40 | 21514.75 | 49610.60 |
| After-26 | 884.79 | 13617.15 | 155322.60 |
| After-38 | 2427.62 | 8147.72 | 79606.74 |
| After-39 | 2513.75 | 29900.44 | 120019.40 |
| After-54 | 1255.79 | 6446.03 | 102599.75 |
| After-57 | 2284.68 | 24717.16 | 167783.10 |
| After-101 | 80.89 | 1102.35 | 13894.65 |
| After-112 | 284.26 | 2310.30 | 180202.29 |
| After-114 | 114.99 | 1235.86 | 18870.81 |
| After-115 | 113.36 | 1007.71 | 32085.88 |
| After-119 | 106.12 | 933.14 | 42201.05 |
| After-139 | 98.93 | 1081.66 | 20656.22 |
| After-75 | 105.97 | 1117.19 | 25499.60 |
| After-83 | 145.55 | 1428.01 | 29704.77 |
| After-90 | 176.18 | 1121.90 | 42202.48 |
| After-91 | 180.36 | 1049.60 | 33029.69 |
| After-103 | 3.30 | 1566.57 | 731.49 |
| After-107 | 5.73 | 3642.67 | 2087.46 |
| After-158 | 2.25 | 1035.35 | 885.98 |
| After-166 | 9.87 | 2518.78 | 2522.29 |
| After-185 | 18.55 | 5053.50 | 7360.04 |
| After-191 | 23.11 | 2810.64 | 11914.16 |
| After-206 | 3.72 | 1776.12 | 741.51 |
| After-233 | 7.05 | 3256.05 | 3210.83 |
| After-259 | 16.08 | 9395.38 | 9208.12 |
| After-281 | 3.09 | 2427.49 | 817.28 |
| After-290 | 12.27 | 2031.58 | 8007.07 |
| After-291 | 8.52 | 5618.71 | 3670.65 |
| After-292 | 8.88 | 2223.16 | 6742.54 |
| After-297 | 15.69 | 6191.35 | 8899.60 |
| After-322 | 130.56 | 23220.99 | 38576.83 |
| Placebo-1 | 249.32 | 462.45 | 88.14 |
| Placebo-10 | 55.63 | 178.83 | 620.64 |
| Placebo-11 | 2.90 | 232.65 | 1108.62 |
| Placebo-13 | 34.37 | 770.62 | 63.37 |
| Placebo-14 | 3.88 | 689.91 | 841.34 |
| Placebo-15 | 22.52 | 126.52 | 345.20 |
| Placebo-16 | 0.67 | 120.56 | 127.39 |
| Placebo-17 | 0.48 | 233.73 | 202.24 |
| Placebo-19 | 14.59 | 2370.23 | 3399.59 |
| Placebo-2 | 0.35 | 167.37 | 70.58 |
| Placebo-21 | 0.28 | 558.81 | 170.33 |
| Placebo-22 | 11.15 | 64.40 | 1373.20 |
| Placebo-23 | 18.06 | 373.08 | 203.23 |
| Placebo-24 | 0.25 | 812.15 | 137.71 |
| Placebo-3 | 0.02 | 0.61 | 0.17 |
| Placebo-4 | 200.17 | 563.91 | 403.89 |
| Placebo-5 | 59.22 | 327.60 | 1941.54 |
| Placebo-6 | 50.47 | 227.77 | 1683.70 |
| Placebo-7 | 0.14 | 144.20 | 27.45 |
| Placebo-8 | 4.29 | 432.10 | 974.32 |
| Placebo-9 | 34.88 | 118.31 | 170.36 |
| Oxygen-1 | 0.47 | 181.40 | 83.65 |
| Oxygen-10 | 0.52 | 8.26 | 119.21 |
| Oxygen-11 | 10.38 | 993.12 | 2289.22 |
| Oxygen-13 | 37.71 | 409.05 | 284.51 |
| Oxygen-14 | 1.54 | 95.99 | 462.88 |
| Oxygen-15 | 0.13 | 101.31 | 36.07 |
| Oxygen-16 | 9.22 | 285.18 | 590.47 |
| Oxygen-17 | 0.44 | 807.83 | 200.78 |
| Oxygen-19 | 297.98 | 2274.53 | 402.90 |
| Oxygen-2 | 0.21 | 508.08 | 27.70 |
| Oxygen-21 | 0.90 | 712.45 | 153.56 |
| Oxygen-22 | 68.81 | 146.12 | 404.07 |
| Oxygen-23 | 0.53 | 336.31 | 53.94 |
| Oxygen-24 | 0.09 | 158.29 | 31.26 |
| Oxygen-3 | 0.10 | 80.81 | 32.81 |
| Oxygen-4 | 45.55 | 164.08 | 4011.74 |
| Oxygen-5 | 0.89 | 260.98 | 537.05 |
| Oxygen-6 | 48.75 | 300.01 | 154.06 |
| Oxygen-7 | 0.24 | 206.27 | 16.86 |
| Oxygen-8 | 58.69 | 264.81 | 3215.08 |
| Oxygen-9 | 0.44 | 131.07 | 209.37 |

Statistical Analysis: Hierarchical clustering was performed to calculate principal component analysis and to generate heatmaps using the R-based tool ClustVisl (Metsalu and Vilo, 2015). Unsupervised hierarchical clustering was performed using Euclidean distance and complete linkage for columns (miRNA value relative to spike RNA) and rows (sample ID). The heat map graphs were re-oriented (columns and rows transposed) for display of the data.

### Example 2: Candidate miRNA Markers of PH

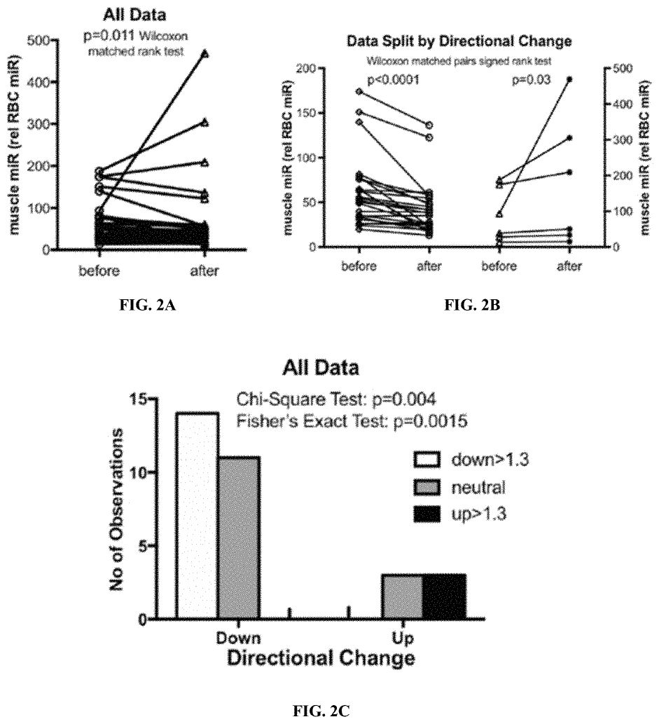

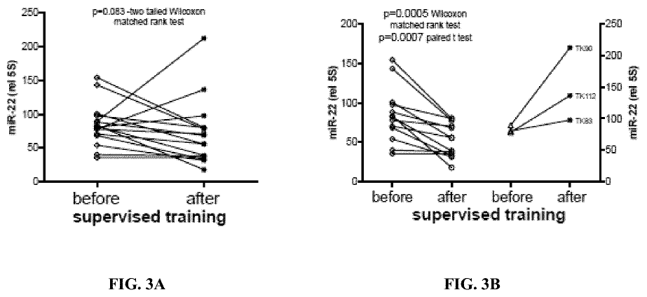

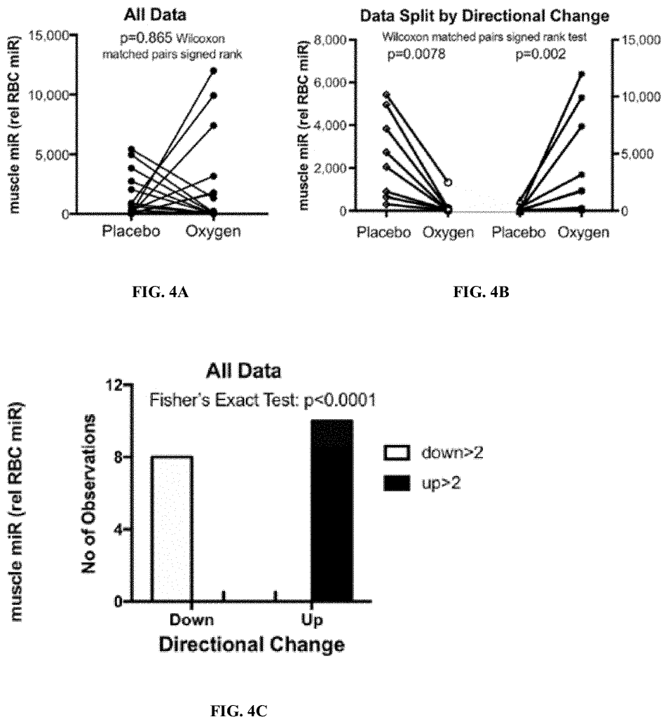

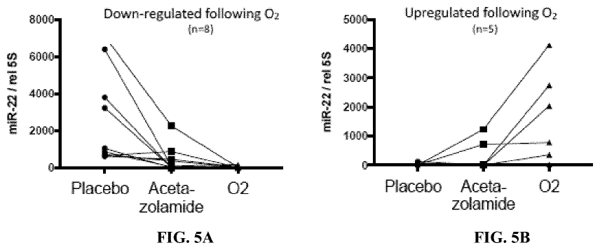

In this experiment, specific circulating miRNAs were identified as markers of pulmonary hypertension. Samples were taken from PH patients who were either subjected to oxygen intake therapy or to exercise training. Serum samples from different types of WHO class I patients, including heritable pulmonary arterial hypertension (hPAH) and idiopathic PAH patients, taken before and after supervised exercise training were analyzed for a variety of miRNAs, including those that control muscle function (e.g., smooth muscle and skeletal muscle cells function) and red blood cell function (FIGS. 2A-2C, TABLE 4, and TABLE 5). The duration of the supervised exercise training was 3 weeks. Serum samples from the patients taken before and after supervised exercise training were also specifically analyzed for the level of miR-22 relative to 5S RNA (FIGS. 3A-3B). Plasma samples from pulmonary hypertension patients treated with nightly oxygen intake or placebo were analyzed for a number of miRNAs including those that control muscle function (FIGS. 4A-4C, TABLE 4, and TABLE 5). Further focused analysis regarded plasma samples from pulmonary hypertension patients treated with nightly oxygen intake, or placebo, or a breath stabilizer (acetazolamide) for the level of miR-22 relative to 5S RNA (FIGS. 5A-5B). For some of the studies, ratios between circulating levels of muscle miR relative to red blood cell (RBC) miR from the plasma or serum samples were determined and statistical analysis calculated based on a Wilcoxon matched pairs signed rank test (FIGS. 2A-2C, FIGS. 3A-3B, and FIGS. 4A-4C). The plasma samples from the pulmonary hypertension patients were obtained before and after 1 week of a therapeutic program (e.g., before and after 1 week of treatment with nightly oxygen intake, or placebo, or a breath stabilizer). A randomized, double blinded cross-over method was used to select patients for the nightly oxygen intake, the placebo, or the breath stabilizer (acetazolamide).

In short, samples were obtained from the patients, RNA was isolated from the plasma or serum samples, and miRNA levels were determined by real time quantitative polymerase chain reaction (RTq-PCR) and/or RNA sequencing (TABLE 4 and TABLE 5). Changes in specific miRNA levels were identified in all studies. Overall, a downward trend or an upward trend was observed depending on the patient and/or the specific miRNAs analyzed (FIGS. 2A-2C, FIGS. 3A-3B, FIGS. 4A-4C, and FIGS. 5A-5B). Unexpectedly, though, the direction of the change was not the same in all studies. For example, 84% of the patients showed a downward trend following exercise training (FIGS. 2A-2C) while 44% of the patients showed a downward trend after receiving oxygen therapy (FIGS. 4A-4C).

The ratio between muscle miR relative to RBC miR decreased in some patients when measured before and after exercise training while the ratio increased in other patients (FIGS. 2A-2C). Similarly, the ratio between muscle miR relative to RBC miR decreased in some patients after oxygen intake while the ratio increased in other patients (FIGS. 4A-4C). Thus, in some patients the muscle miR was upregulated while in other patients the muscle miR was downregulated. In addition, this data showed that in some patients, the RBC miR was upregulated in some patients while the RBC miR was downregulated in other patients (FIGS. 2A-2C and FIGS. 4A-4C).

FIGS. 3A-3B and FIGS. 5A-5B show miR-22 levels relative to levels of 5S RNA (at 10,000 U). The ratio between miR-22 levels relative to 5S RNA decreased in some patients after exercise training (FIGS. 3A-3B) or after oxygen therapy (FIG. 5A) while the ratio increased in other patients after exercise training (FIGS. 3A-3B) or after oxygen therapy (FIG. 5B). Thus, in some patients the miR-22 level was upregulated after exercise training (FIGS. 3A-3B) or after oxygen therapy (FIG. 5B) while the miR-22 level was downregulated in other patients (FIGS. 3A-3B and FIG. 5A). In five (5) patients, the miR-22 levels were increased in the acetazolamide treated group only (data not shown). This experiment showed that miRNAs that control muscle function (e.g., miR-22) and miRNAs that control erythrocyte function (e.g., miR-451a) have a role in the processes that control the clinical outcome of PH because all interventions tested (e.g., exercise training, nightly oxygen, breath stabilizer) had a significant positive effect on PH.

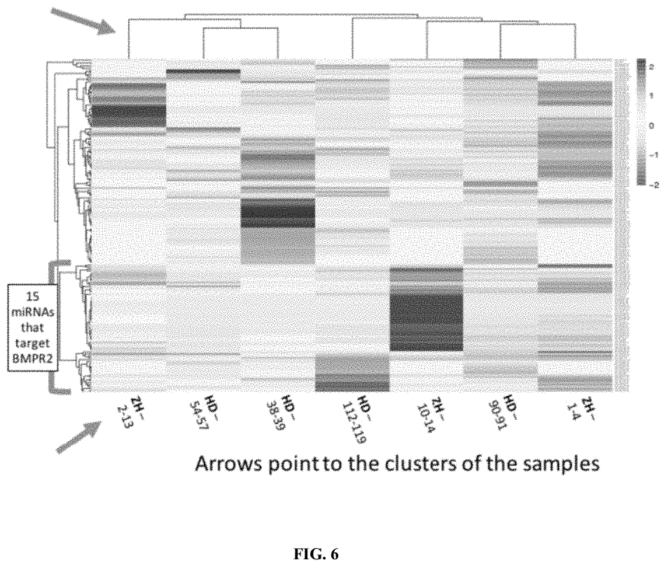

The serum and plasma samples were further subjected to a qPCR array of 150 miRNAs (FIG. 6). In short, q-PCR was performed and cycle threshold (Ct) values relative to Spiked synthetic RNA (UniSp6 RNA) were determined. Fold change values were then calculated (e.g., after vs. before exercise training or nightly oxygen vs. placebo treatment) followed by a heatmap cluster analysis (ClustVis). The heatmap analysis of the miRNA array unexpectedly showed that some of the samples from the different studies (e.g., exercise training and oxygen therapy studies) clustered together (FIG. 6). The heatmap analysis showed that multiple miRNAs targeted the bone morphogenetic protein receptor 2 (BMPR2), a risk factor for the development of pulmonary hypertension, and were found to be clustered together (FIG. 6). In contrast, many of the miRNAs that target PH neutral genes, for example miRNAs that target p53 (a cancer gene), were found in multiple clusters throughout the heatmap (FIG. 6). This experiment showed that miRNAs that target BMPR2 can be used as markers for PH. In addition, this experiment showed that miRNAs that target BMPR2 were downregulated after exercise and/or oxygen intervention. In particular, the miRNAs that were found to be downregulated (in heatmap clusters) were hsa-miR-100-5p, hsa-miR-93-5p, hsa-miR-92b-3p, hsa-miR-20a-5p, hsa-miR-17-5p, hsa-miR-130a-3p, hsa-miR-27a-3p, hsa-miR-106b-5p, hsa-miR-19a-3p, hsa-miR-7-5p, hsa-miR-20b-5p, hsa-miR-19b-3p, hsa-miR-32-5p, hsa-miR-192-5p, and hsa-miR-215-5p. This experiment showed that miRNAs (circulating levels of miRNAs) that target BMPR2 can be affected by treatment (e.g., exercise training and/or oxygen intervention).

### Example 3: Identification of miRNAs in PH Patients

Paired bio-banked serum (n=31) or plasma (n=21) samples from PH patients who received exercise training intervention or nightly oxygen/placebo intake, respectively, were analyzed. Samples were obtained before and after exercise training or before and after oxygen/placebo intake (e.g., nightly oxygen intake). The nightly oxygen and placebo treatment groups were determined based on a randomized, double blinded cross-over manner. Each patient was randomly assigned to placebo (air) or oxygen first, and then crossed-over to receive the other intervention. The PH patients in the exercise training intervention group were subjected to 3 weeks of supervised exercise training. The baseline characteristics of the PH patients are listed in TABLE 6 and TABLE 7. In short, the samples were used for RNA sequencing and quantitative PCR to identify miRNAs in the samples that showed changes in expression levels. The focus was to identify miRNAs based on the magnitude of the change, irrespective of the direction of the change (increased or decreased miRNA levels). A number of miRNAs were identified including miR-22-3p, miR-451a, and miR-21-5p. Endogenous reference miRNAs that were predicted to show little, if any, variations among the samples were also analyzed. One identified reference miRNA of this disclosure is miR-451a (in some patients). However, in other patients, miR-451a changed with the intervention.

TABLE 6
Characteristics of sample populations (means ±
standard error of the mean (SEM) are indicated)

| | | Supervised exercise | Nightly oxygen |
|---|---|---|---|
| | | treatment program group | treatment group |
| | | at baseline | at baseline |
| Number of Pairs | | 31 | 21 |
| Age | | 50.68 ± 2.96 | 65.14 ± 2.21 |
| Gender | Female | 21 | 13 |
| | Male | 10 | 8 |
| NYHA | Class II | 16 | 5 |
| | Class III | 9 | 16 |

| | | |
|---|---|---|
| BMI (kg/m²) | 28.28 ± 1.24 | 27.33 ± 0.89 |
| 6 MWD (m) | 459.50 ± 23.03 | 438.30 ± 19.79 |
| PH with additional diagnoses | 87% | 100% |
| Mean PA pressure (mmHg) | 50.54 ± 3.38 | 40.50 ± 3.80 |

NYHA: New York Heart Association; BMI: body mass index; 6 MWD: 6 minutes walking distance; Mean PA (pulmonary artery) pressure. Of the patients with missing NYHA classification (n = 6) the mean pulmonary artery pressures at rest were between 50 mmHg to 80 mmHg in three patients, and 40 mmHg in one patient.

TABLE 7
Diagnoses of PH patients

| | Supervised exercise | | Nightly oxygen | |
|---|---|---|---|---|
| | treatment program group | | treatment group | |
| Total number of PH patients | 31 | (100%) | 21 | (100%) |

Primary diagnosis

| | | | | |
|---|---|---|---|---|
| Idiopathic PAH | 19 | (61%) | 12 | (57%) |
| Heritable PAH | 7 | (22.6%) | | |
| Associated PAH | | | | |
| connective tissue disease | 2 | (6.5%) | 1 | (4.8%) |
| portal hypertension | 2 | (6.5%) | 1 | (4.8%) |
| congenital heart disease | 1 | (3.2%) | | |
| Chronic thromboembolic PH | | | 7 | (33%) |

Additional diagnosis

| | | | | |
|---|---|---|---|---|
| Number of patients who had | 27 | (87%) | 21 | (100%) |
| additional diagnoses | | | | |
| Transient embolic episodes | 6 | (19% | | |
| of the pulmonary artery | | | | |
| Peripheral vascular disease | 9 | (29%) | 8 | (38%) |
| (hypertension, subdural | | | | |
| hematoma, arterial blockage, | | | | |
| chronic venous disease) | | | | |
| Chronic lung disease (COPD, | 8 | (26%) | 6 | (28%) |
| airway hyperreactivity, | | | | |
| airway obstruction, asthma) | | | | |
| Left heart disease and/or | 7 | (23%) | 6 | (28%) |
| heart rhythm abnormalities | | | | |
| Anemia, or iron therapy | 8 | (26%) | 2 | (9.5%) |
| Auto-immune disease (e.g., | 3 | (9.7%) | 2 | (9.5%) |
| insulin dependent diabetes, | | | | |
| or autoimmune thyroiditis) | | | | |
| Kidney disease | 5 | (16%) | 5 | (24%) |
| Tumor | 2 | (6.5%) | 1 | (4.8%) |

One primary diagnosis per patient. All patients received PH specific medication. One or more additional diagnosis per patient. The patients received diagnosis specific medication as needed.

Human plasma and serum samples: De-identified, bio-banked plasma and serum samples from the PH patients were analyzed. Paired, banked serum samples obtained either before or after the supervised exercise training program were analyzed by isolating RNA from the serum samples followed by miRNA detection by real time PCR. The plasma samples from PH patients subjected to nightly oxygen/placebo treatment were similarly analyzed. Plasma samples were obtained after 1-week of nightly oxygen treatment or placebo treatment.

RNA isolation from plasma or serum samples: About 0.2 mL of plasma is usually sufficient to isolate enough RNA (200 ng) for analyzing more than 10 miRNA species using single primers and quantitative RT-PCR, or for loading on a 384 well PCR array. Total RNA purification was performed in 200 μL of plasma and/or serum samples from either the PH patients subjected to a nightly oxygen/placebo treatment or from PH patients subjected to exercise treatment. Total RNA was purified using miRNAeasy Mini Kit according to manufacturer's protocol (Qiagen, Valencia Calif.) and eluted into 35 μL of water. During the RNA isolation process, 1 μL of UniSp6 RNA Spike-in template (representing 10⁸ copies) was added after the lysis and the homogenization steps. The Spike RNA was used as an exogenous reference. The UniSp6 RNA Spike-in template was provided with the miRCURY LNA™ Universal cDNA synthesis kit II (Exiqon, Woburn, Mass.). Synthetic Spike RNA (synthetic control template, 10⁸ copies/μL) was added to each sample during the RNA isolation procedure for quantification purposes. The RNA concentration was determined by Nanodrop and about 20 ng of RNA were reverse transcribed to obtain cDNA (Exiqon-Qiagen kit). MiRNA expression levels were determined by qPCR using LNA primers (Exiqon-Qiagen).

For all human plasma samples, a heparinase step was performed following RNA purification. Thirty nanograms of RNA were treated with the following: 0.3 U of heparinase (H2519-178 50UN, Sigma-Aldrich, St. Louis, Mo.) and 22 U RNAse inhibitor (Invitrogen) re-suspended in 1×RT buffer from miRCURY LNA™ Universal cDNA synthesis kit II, for 1 hour at 25° C. to remove heparin. Reverse transcriptase reaction was performed with miRCURY LNA™ Universal cDNA synthesis kit II according to manufacturer's protocols. RNA concentrations were measured with DS-11 spectrophotometer (DeNovix, Wilmington, Del.).

MiRNA expression: Real time PCR was performed in triplicate with 0.1 ng of cDNA per reaction using a 7900HT Fast Real-Time PCR instrument (Applied Biosystems/Life Technologies, Grand Island, N.Y.) in a 10 μL volume. The PCR reactions were run with LNA-modified primers and SYBR Green master mix (Exiqon, Denmark/now Qiagen) in 384-well plate under the following conditions: 95° C. for 10 min, followed by 50 cycles of 95° C. for 10 s and 60° C. for 1 min, followed by a hold at 4° C. The following LNA-modified primers were used: hsa-miR-451a (target 5′AAACCGUUACCAUUACUGAGUU; SEQ ID NO: 60); hsa-miR-22-3p (target 5′AAGCUGCCAGUUGAAGAACUGU; SEQ ID NO: 10); hsa-miR-21-5p (target 5′UAGCUUAUCAGACUGAUGUUGA; SEQ ID NO: 9); and Spike6 (target the synthetic Spike RNA, UniSP6, supplied in the cDNA kit).

Raw data were then analyzed with SDS Relative Quantification Software version 2.4.1 (Applied Biosystems) to determine cycle threshold (Ct). The miRNA values were calculated with the following formula: [Ct of miRNA reference(s)−Ct of miRNA determinant(s)] multiplied by the power of 1.98, and then multiplied by 10,000. The miRNA values were calculated without knowledge of the characteristics of the sample donors.

Statistical analysis: Statistical analysis and graphs were generated using Prism 6 (GraphPad, La Jolla, Calif.). Data (e.g., group comparisons) were analyzed with the two-tailed, independent Mann-Whitney U test or the Wilcoxon matched pairs signed rank test. Correlations were calculated with the Spearman's Rank Correlation test. A p-value <0.05 was considered to be statistically significant.

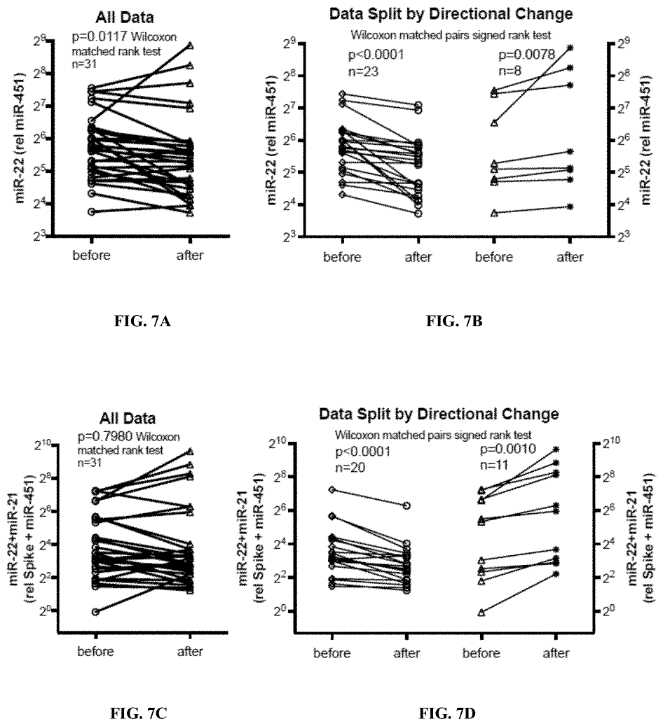

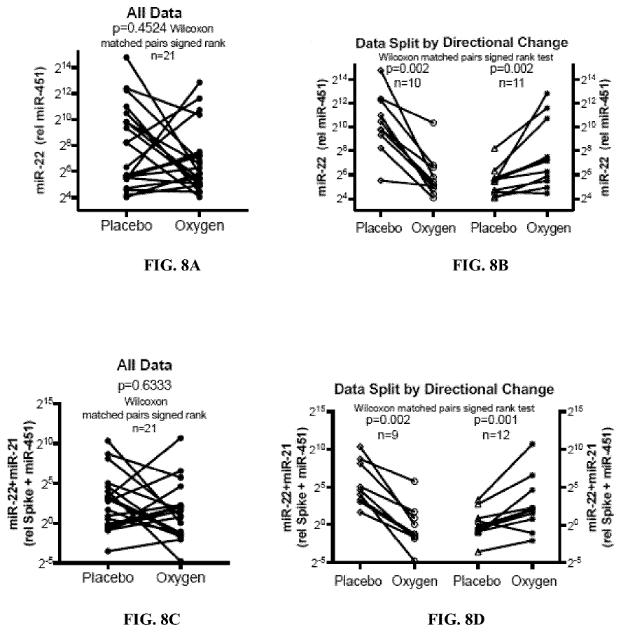

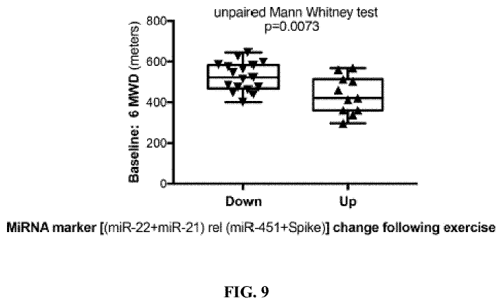

Results: The levels of miRNAs that control muscle function were compared to the levels of miRNAs that control erythrocyte function. The levels of miRNAs that control muscle function or that control erythrocyte function were also compared to Spike RNA (exogenous reference). Further, the levels of miRNAs that control muscle function were compared to the levels of Spike RNA (exogenous reference) combined with the levels of miRNAs that control erythrocyte function. The miRNA levels were determined before and after oxygen/placebo treatment or before and after exercise training. MiRNA comparison levels of this experiment included: miR-22-3p relative to miR-451a; miR-21-5p relative to miR-451a; miR-22-3p relative to Spike RNA; miR-21-5p relative to Spike RNA; miR-451a relative to Spike RNA; the combination of miR-21-5p and miR-22-3p relative to the combination of miR-451a and Spike RNA; the combination of miR-21-5p, miR-22-3p, and miR-451a relative to Spike RNA; the combination of miR-21-5p and miR-22-3p relative to miR-451a; and the combination of miR-21-5p and miR-22-3p relative to Spike RNA. The miRNA levels and the fold change values were determined (TABLE 8, FIGS. 7A-7D, and FIGS. 8A-8D).

Results showed that the fold change determined for miR-22-3p relative to Spike RNA correlated significantly with the fold change determined for miR-21-5p relative to Spike RNA, in both the exercise training and the oxygen intervention study groups (TABLE 8). The ratio of miR-22-3p relative to miR-451a and the ratio of the combination of miR-22-3p and miR-21-5p relative to the combination of miR-451a and Spike RNA (i.e., (miR-22-3p+miR-21-5p)/(miR-451a+Spike RNA)) were also determined before and after supervised training or before and after oxygen/placebo intake (FIGS. 7A-7D and FIGS. 8A-8D, respectively).

Measurements of miR-22-3p/miR-451a values in serum from patients receiving exercise training showed that the values were significantly decreased in 74.2% of the samples following intervention and significantly increased in the remainder (25.8%) (FIG. 7A and FIG. 7B). In samples obtained after exercise intervention, a higher composite miRNA value, made of miR-22-3p and miR-21-5p/miR-451a and spike RNA, was significantly decreased in 65% of the samples and significantly increased in 35% of the samples (FIG. 7C and FIG. 7D). The samples in the exercise training group were then separated based on the direction of the fold change (FIG. 7B and FIG. 7D).

The data from the nightly oxygen intervention study showed that higher miRNA marker values were detected as compared to the values measured for the exercise intervention study (FIGS. 8A-8D compared to FIGS. 7A-7D). This difference could be due to the materials that were used, for example, serum was used in the exercise intervention study (FIGS. 7A-7D) while plasma was used in the oxygen intervention study (FIGS. 8A-8D). As observed in the exercise intervention study, the oxygen intervention study showed that significant changes in the direction of the miRNA values varied depending on the subject, with approximately half of the patients demonstrating decreased (downward change) and the other half demonstrating increased (upward change) miRNA marker values (FIGS. 8A-8D). A similar trend was observed for both the ratio between miR-22-3p relative to miR-451a and the combination of miR-22-3p and miR-21-5p relative to the combination of miR-451a and Spike RNA (FIGS. 8B and 8D). Samples that had a downward change in the miRNA marker following either intervention (i.e., exercise training study or the oxygen/placebo intake study) originated from patients who had a significantly higher 6-minute walking distance at baseline (mean difference of 90 m or 80 m following exercise or oxygen intervention, respectively) when compared to samples that had an upward change in the miRNA marker. This fold change difference can be indicative of different molecular pathways that cause PH in the patients (e.g., PH endotypes).

Further, a composite miRNA marker was also analyzed. The miR-22-3p and miR-21-5p levels were combined to diminish the significance of potential technical variations (minute pipetting errors for example) in the qPCR quantification values. The composite miRNA fold change of miR-22-3p and miR-21-5p was calculated compared to the composite miRNA levels of miR-451a and Spike RNA. Spike RNA was used as an invariant component because the same number of copies (10⁸) of Spike RNA was added to each 200 μL of sample used for the RNA isolation. Data showed that the composite miRNA levels of miR-22-3p and miR-21-5p relative to miR-451a and Spike RNA were significantly correlated with the values for miR-22-3p relative to miR-451a in both the exercise training and the oxygen intervention studies (TABLE 8, FIGS. 7A-7D, and FIGS. 8A-8D).

TABLE 8
Correlation between different types of miRNA measurement readouts
A. MiRNA marker change following exercise training intervention (n = 31 pairs)

| X-Variable | miR-22/miR-451 | miR-22/miR-451 | miR-22/miR-451 | miR-22/Spike | miR-451/Spike | miR-22/miR-451 |
|---|---|---|---|---|---|---|
| Y-Variable | miR-22/Spike | miR-451/Spike | miR-21/Spike | miR-21/Spike | miR-21/Spike | miR-22 + miR-21/ |
| | | | | | | miR-451 + Spike |
| Spearman's R | 0.3156 | −0.3480 | 0.0970 | 0.7040 | 0.6290 | 0.5989 |
| P-value | 0.0838 | 0.0550 | 0.6037 | <0.0001 | 0.0002 | 0.0004 |

B. MiRNA marker change following nightly oxygen intervention (n = 21 pairs)

| | | | | | | |
|---|---|---|---|---|---|---|
| X-Variable | miR-22/miR-451 | miR-22/miR-451 | miR-22/miR-451 | miR-22/Spike | miR-451/Spike | miR-22/miR-451 |
| Y-Variable | miR-22/Spike | miR-451/Spike | miR-21/Spike | miR-21/Spike | miR-21/Spike | miR-22 + miR-21/ |
| | | | | | | miR-451 + Spike |
| Spearman's R | 0.7636 | −0.2558 | 0.3143 | 0.5221 | 0.2494 | 0.8688 |
| P-value | <0.0001 | 0.2630 | 0.1653 | 0.0152 | 0.2757 | <0.0001 |

Data in TABLE 8 was calculated using the Spearman's rank correlation test on the fold-change of the miRNAs, after exercise training relative to baseline, or after oxygen intake relative to placebo. For FIGS. 7A-7D and FIGS. 8A-8D, the samples from the patients before and after intervention were compared using the Wilcoxon matched pairs signed rank test. A p<0.05 was considered significant.

### Example 4: Comparison of miRNA Levels to the Distance Obtained from a 6 MWD from Patients Subjected to Exercise Training or Oxygen Intervention

To understand whether the fold change difference observed in Example 2 above could indicate different molecular pathways to PH, the results of the 6-minute walking distance (6 MWD) test were analyzed.

Exercise training study: a significant difference in the 6-minute walking distance was observed when the miRNA directional changes were compared to baseline disease characteristics (FIG. 9). Samples that showed a decreased directional change (or down regulation) in the miRNA markers (e.g., miR-22-3p+miR-21-5p relative to miR-451a+Spike RNA) (FIG. 7D) correlated with patients who had a significantly longer 6-minute walking distance at baseline (FIG. 9 and TABLE 9). In other words, samples from patients who were able to walk longer distances at baseline during the 6 MWD test displayed a decrease in the ratio of miRNA markers (e.g., miR-22-3p+miR-21-5p relative to miR-451a+Spike RNA) after exercise training as compared to before exercise training (FIG. 9). FIG. 9 shows box-plots with whiskers and individual points representing the 6-minute walking distance (in meters) measured at baseline from patients in the exercise training intervention study. The data were first grouped based on the directional change of the levels of miR-22-3p+miR-21-5p relative to Spike-RNA+miR-451a (×10,000), and then compared to the distance walked by each subject prior to and after exercise training intervention. The groups were compared using an unpaired, 2-tailed Mann-Whitney test (p<0.05 was considered significant). For this analysis, samples from 3 patients who had a body mass index (BMI) greater that 40 kg/m² and who achieved a 6-minute walking distance between 150-190 meters were removed. The p value was 0.0074 (t-test).

TABLE 9
Baseline 6-minute walking distance in the exercise intervention
study: comparison with directional change of miRNA markers

| | Down regulated | Up regulated |
|---|---|---|
| N | 17 | 11 |
| Gender (female/male) | 13/4 | 6/5 |
| Age (Y) | 47.6 ± 4.1 | 52.4 ± 5.1 |
| BMI (kg/m²) | 27.10 ± 1.20 | 26.20 ± 1.67 |
| 6 MWD (m) | 525.4 ± 17.2 | 435.5 ± 27.9 |
| NYHA class (II/III) | 11/3 | 5/4 |
| Mean PA pressure (mmHg) | 50.2 ± 4.4 | 53.7 ± 6.4 |
| | (n = 15) | (n = 10) |

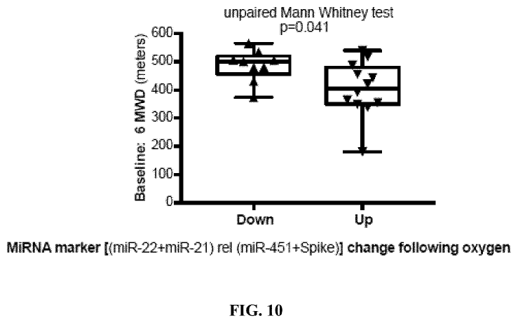

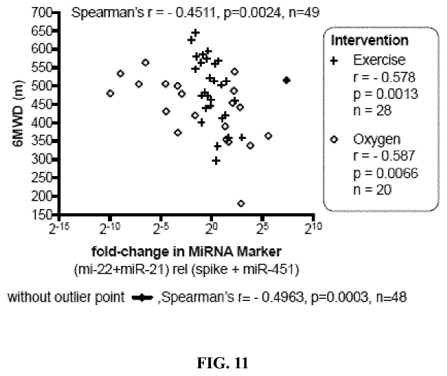

Oxygen therapy study: the group of patients whose miRNA values were significantly decreased was compared to the group of patients whose miRNA values were significantly increased following oxygen intervention. A significant difference in the 6-minute walking distance recorded at baseline was observed between the groups (FIG. 10 and TABLE 10). The p value was 0.034 (t-test). FIG. 10 shows box-plots with whiskers and individual points representing the 6-minute walking distance (6 MWD, in meters) measured at baseline from patients in the oxygen intervention study. The data were grouped based on the directional change of the combination of miR-22-3p+miR-21-5p relative to the combination of Spike RNA+miR-451a (×10,000) in patients treated with either oxygen or placebo. The groups were compared using an unpaired, 2-tailed Mann-Whitney test (p<0.05 was considered significant). Similar to the data obtained from the exercise intervention study (FIG. 9), the group that had an upward change in the ratio of miRNAs following oxygen intervention had a significantly lower 6-minute walking distance at baseline (FIG. 10).

Further, in both intervention studies, a higher percentage of males showed upregulation in the ratio of the miRNA markers (TABLE 9 and TABLE 10). To confirm further the possibility that the 6-minute walking distance at baseline could be correlated with the directional fold-change of the miRNA markers following intervention, the ratio of the combination of miR-22-3p and miR-21-5p relative to the combination of miR-451a and Spike RNA was calculated in both serum and plasma samples (FIG. 11). In short, the fold change in miR-22-3p+miR-21-5p relative to Spike-RNA+miR-451a (×10,000) levels were measured from patients and plotted against each patient's 6-minute walking distance in meters (m) at baseline. The fold change was measured for patients subjected to exercise training (before versus after exercise training) and for patients who received oxygen treatment or placebo (FIG. 11). The data from the exercise training intervention study were plotted with + symbols, and the data from the nightly oxygen intervention study were plotted with diamond symbols (FIG. 11). The correlation was calculated with Spearman's rank correlation test (p<0.05 was considered significant). Samples from 3 patients who had a body mass index (BMI) greater that 40 kg/m² and who achieved 6-minute walking distances between 150-190 meters were omitted. The analysis showed a significant correlation between the exercise and the oxygen groups when using Spearman's rank correlation test (FIG. 11). This experiment showed that the change in the miRNA levels were linked with PH endotype (causative physiological or molecular pathways) of the patients.

TABLE 10
Baseline 6-minute walking distance in the oxygen intervention
study: comparison with directional change of miRNA markers

| | Down regulated | Up regulated |
|---|---|---|
| N | 9 | 12 |
| Gender (female/male) | 6/3 | 7/5 |
| Age (Y) | 66.44 ± 2.76 | 64.17 ± 3.33 |
| BMI (kg/m²) | 27.47 ± 1.49 | 27.24 ± 1.15 |
| 6 MWD (m) | 485.8 ± 18.7 | 402.7 ± 28.0 |
| NYHA class (II/III) | 2/7 | 3/9 |
| Mean PA pressure (mmHg) | 39.56 ± 4.82 | 44.86 ± 4.61 |
| | (n = 8) | (n = 11) |

### Example 5: Cattle Model of High Altitude Induced PH

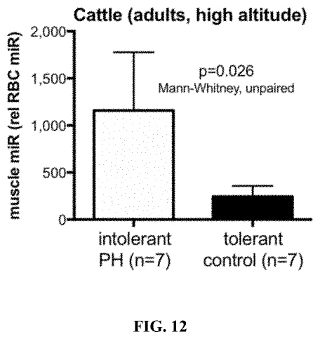

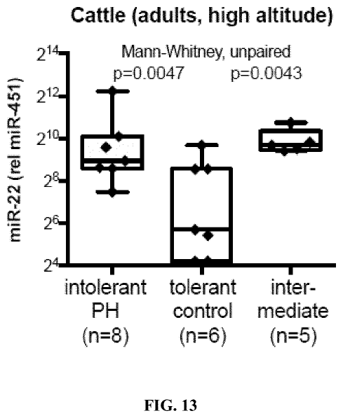

The miRNAs that control muscle function (e.g., skeletal, heart, and/or smooth muscle) and/or the miRNAs that control erythrocyte function were tested in animal models. Specifically, the miRNAs (e.g., miR-22-3p, miR-21-5p, and miR-451a) that showed a significant change in expression levels between the samples taken pre and post treatment in humans (e.g., before and after oxygen/placebo intake or exercise training), were further analyzed in animal models (TABLE 11). Samples were taken before and after exercise training or before and after nightly oxygen/placebo intake. Bio-banked Plasma samples (n=20) from cattle that were tolerant to high altitude (PAP≤50 mmHg), cattle that had developed pulmonary hypertension (PAP≥79 mmHg), and cattle experiencing intermediate PAP (50 mmHg<PAP<79 mmHg) were analyzed by isolating RNA from the plasma samples and measuring miRNA levels using PCR. Results showed that a significant difference in the level of a muscle miR relative to an RBC miR was observed between plasma samples from tolerant (control) cattle as compared to plasma samples from intolerant cattle (FIG. 12). Plasma samples from cattle that developed PH in high altitude had significantly higher miR-22-3p/(relative to) miR-451a values when compared to control cattle tolerant to high altitude. The data showed significant decrease in miR-22-3p levels relative to miR-451a levels in the plasma samples from tolerant (control) cattle, compared to the intolerant and intermediate groups (FIG. 13). MiR-22-3p and miR-451a were found to be of molecular interest in PH, particularly in PH associated with oxygen consumption. TABLE 11 shows the miR-22-3p and miR-452a levels in control (cattle tolerant to high altitude), intermediate, and intolerant cattle samples. Ct values relative to spike RNA were calculated and multiplied by 10.

TABLE 11
Plasma miRNA levels determined relative to spike RNA from
cattle that were tolerant to high altitude (control), from
cattle that were intolerant to high altitude, and from cattle
that exhibited intermediate response to high altitude

| Cattle Sample ID | miR-22-3p | miR-451a |
|---|---|---|
| Control (tolerant)-1 | 3.59 | 95.65 |
| Control (tolerant)-2 | 1.85 | 435.72 |
| Control (tolerant)-3 | 0.30 | 68.96 |
| Control (tolerant)-4 | 0.63 | 35.94 |
| Control (tolerant)-5 | 1.39 | 153.92 |
| Control (tolerant)-6 | 3.45 | 91.38 |
| Intermediate-1 | 3.62 | 44.07 |
| Intermediate-2 | 72.03 | 419.97 |
| Intermediate-3 | 37.33 | 412.62 |
| Intermediate-4 | 2.98 | 40.79 |
| Intermediate-5 | 15.09 | 221.12 |
| Intolerant-1 | 2.06 | 52.03 |
| Intolerant-2 | 30.04 | 325.69 |
| Intolerant-3 | 1.41 | 79.11 |
| Intolerant-4 | 277.06 | 576.95 |
| Intolerant-5 | 37.33 | 483.93 |
| Intolerant-6 | 42.59 | 389.87 |
| Intolerant-7 | 3.40 | 68.91 |
| Intolerant-8 | 21.53 | 559.55 |

Cattle plasma samples: Plasma samples were analyzed from groups of cattle that were kept at high altitude (2300 meters and higher). One group of cattle, with pulmonary artery pressures (PAP) of 50 mmHg or lower, remained healthy and tolerant to the altitude. Another group of cattle, with PAP of 79 mmHg and higher, exhibited signs of intolerance to high altitude. Some cattle exhibited an intermediate response, PAP of 50-79 mmHg. The average PAP for intolerant cattle was 91.5±3.6 mmHg, for tolerant cattle was 39.3±1.8 mmHg, and for intermediate cattle was 59.4±4.1 mmHg. The groups were compared using an unpaired, 2-tailed Mann-Whitney test (p<0.05 was considered significant).

RNA isolation from cattle plasma samples: Cattle RNAs were isolated in a similar manner as that described in Examples 1-3, except that a dialysis step was added prior to the RNA isolation step as the cattle samples can contain citrate and heparin. In short, 200 μL of sample in a dialysis tube (Slide-A-Lyser Mini Dialysis Unit, 2000 MWCO; ThermoFisher Scientific, Waltham, Mass.) were dialyzed against 200 mL of 1×TE (10 mM Tris-HCl pH 8.0, 1 mM EDTA) at room temperature for 1 hour. MiRNAeasy Mini Kit extraction was then performed, 10⁸ copies of UniSp6-Spike RNA were added, and a heparinase treatment followed. Reverse transcriptase reaction was performed with miRCURY LNA™ Universal cDNA synthesis kit II according to manufacturer's protocols and RNA concentrations were measured with DS-11 spectrophotometer (DeNovix, Wilmington, Del.).

Results: The cattle plasma samples showed miRNA marker values in a similar range as those obtained from the human plasma samples from the oxygen treated study (FIG. 13 compared to FIGS. 8A-8B). This cattle model of high-altitude-induced PH showed that the circulating levels of muscle miR relative to erythrocyte (RBC) miR can act as markers for PH.

### Example 6: Detection of miRNA Via Rolling Circle Amplification (RCA)

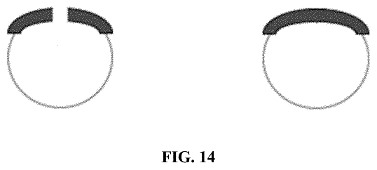

For this experiment, detection of miRNAs in body fluids was performed via rolling circle amplification followed by miRNA specific ligation. Pooled RNA from the serum/plasma samples were used as a source of miRNAs for rolling circle amplification. The RNA samples were spiked with C. elegans miR-39-3p (e.g., at a specific molar ratio of C. elegans miRNA to 10 ng of human RNA). Linearized backbone DNAs (e.g., padlock probes) were designed to specifically recognize particular miRNAs. The padlock probes annealed to the miRNAs were circularized upon addition of DNA ligase (e.g., T7 ligase or SplintR ligase) (FIG. 14). After ligation, the annealed miRNA served as a primer for extension by phi29 DNA polymerase, thereby producing DNA products containing multiple copies of the miRNA sequence.

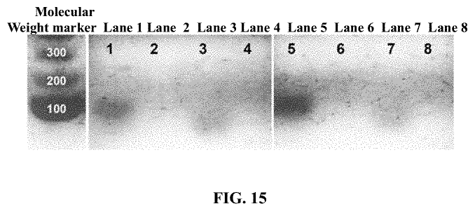

In short, the ligation reaction was performed as follows: 1 μL of serum/plasma RNA (final concentration of 10 ng) and 10 μL of 10 μM backbone DNA (final concentration of 0.1 nM) were incubated at 85° C. for 2 min and then placed on ice. The serum/plasma RNA was spiked with C. elegans miR-39-3p prior to the final steps of RNA isolation. 1 μL of SplintR (NEB M0375S; final concentration of 25 U), 2 μL of 10× buffer, and 6 μL of H₂O were then added to the reaction. The reaction was incubated at 37° C. for 10 min followed by 65° C. for 20 min and placed on ice (or −20° C.). The amplification reaction was performed as follows: 1 μL of the ligation reaction, 2 μL of 10× buffer, 0.4 μL of 10 mg/mL BSA (bovine serum albumin), 0.4 μL of 10 mM dNTPs, 0.5 μL of phi29 (NEB M0269S), and 15.7 μL of H₂O were added together and incubated at room temperature for one day (about 24° C. for approximately 24 hours). The reaction was then run on a gel and stained with ethidium bromide (FIG. 15). FIG. 15 shows the rolling circle amplification reactions performed in this experiment and the molecular weight markers used (100-300 bases). The reaction in lane 1 contained RNA and backbone DNA (for C. elegans miR-39-3p); lane 2 was the negative control with H₂O instead of the RNA and backbone (i.e., 1 μL of RNA and 10 μL of miR-39-3p-backbone DNA were replaced with 11 μL of H₂O instead); lane 3 contained backbone DNA (for C. elegans miR-39-3p) without RNA (i.e., 1 μL of RNA was replaced with H₂O); lane 4 contained RNA without backbone DNA (i.e., 10 μL of backbone DNA was replaced with H₂O); lane 5 contained RNA and backbone DNA (for human miR-451a); lane 6 was the negative control with H₂O instead of the RNA and backbone (i.e., 1 μL of RNA and 10 μL of backbone DNA were replaced with 11 μL H₂O instead); lane 7 contained backbone DNA (for human miR-451a) without the RNA (i.e., 1 μL of RNA was replaced with 1 μL of H₂O); and lane 8 contained RNA without backbone DNA (i.e., 10 μL of backbone DNA specific for miR-451a was replaced with 10 μL of H₂O). Amplification was initiated by the presence of backbone alone (FIG. 15, lanes 3 and 7), possibly because the backbone DNA contained some already-circularized DNA (at 1:100,000 or 1:1,000,000 frequency). However, reactions that contained both the RNA and the backbone (FIG. 15, lanes 1 and 5) showed higher band intensity and higher molecular weight bands compared to the reactions that contained the backbone alone (FIG. 15, lanes 3 and 7). This experiment showed that the rolling circle amplification was dependent on the presence of specific miRNAs (e.g., either human miR-451 or the spiked-in C. elegans miR-39-3p).

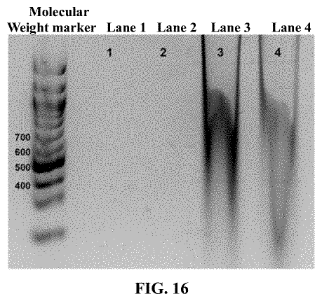

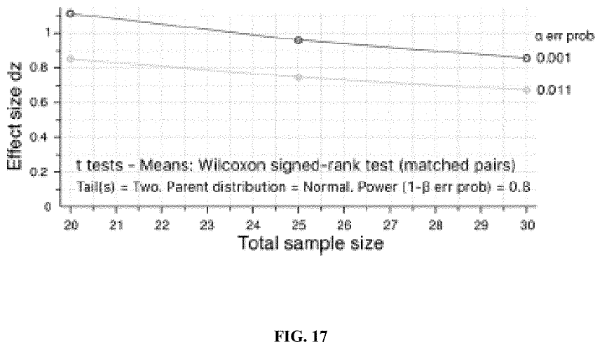

To determine whether rolling circle amplification could produce amplicons from the backbone DNA sequences, amplification using a commercially-available kit was used (GE Healthcare Life Sciences 25-6601-24) (e.g., to initiate amplification with hexamer primers to amplify DNA (all DNA)). For the ligation reaction, 1 μL of serum/plasma RNA (final concentration of 10 ng) and 1 μL of 10 μM backbone DNA (final concentration of 0.01 nM) were added together and incubated at 85° C. for 2 min, then placed on ice. 0.1 μL of SplintR (NEB M0375S; final concentration of 2.5 U), 2 μL of 10× buffer, and 15.9 μL of H₂O were then added to the reaction. The reaction was incubated at 37° C. for 10 min followed by 65° C. for 20 min and ice (or −20° C.) prior to amplification. The amplification reaction was performed according to manufacturer's protocol (GE Healthcare Life Sciences 25-6601-24). In short, 5 μL of the ligation reaction, 5 μL of H₂O, and 10 μL of denaturing buffer were added together and incubated at 95° C. for 3 min then cooled on ice. The freeze-dried Ready-To-Go GenomiPhi V3 cake (provided in the kit) containing DNA polymerase, random hexamers, nucleotides, salts and buffers was reconstituted (on ice) with the denatured template DNA. 20 μL of denatured DNA template was added to the cake and subjected to DNA amplification (25° C. for 16 hours). The samples were then run on a gel and stained with ethidium bromide (FIG. 16). Lane 1 of FIG. 16 was the negative control and contained H₂O instead of RNA and backbone (i.e., 1 μL of RNA and 1 μL of backbone DNA were replaced with 2 μL H₂O); lane 2 contained RNA without the backbone (i.e., 1 μL of backbone DNA was replaced with 1 μL H₂O); lane 3 contained backbone DNA (e.g., backbone DNA for miR-451a) without RNA (i.e., 1 μL of RNA was replaced with 1 μL H₂O); lane 4 contained backbone DNA and RNA (e.g., contained RNA and backbone DNA for miR-451a). This experiment showed that the backbone DNA was highly amplified. The rolling circle amplification resulted in abundant DNA from a reaction using backbone alone (FIG. 16, lane 3) and from a reaction using both backbone and RNA (FIG. 16, lane 4).

This experiment also evaluated the use of circularized probes for rolling circle amplification. One approach is to perform rolling circle amplification using an annealing step containing the RNA/miRNA and probe followed by an amplification step performed at room temperature (or up to about 30° C.) for 2 hours or less (or from between 1 hour to overnight). Primers specific for an miRNA are added and qPCR analysis of the pre-amplicon is performed. For this experiment, single probe specific for one miRNA or a mixture of multiple probes specific for multiple miRNAs can be used. The annealing reaction is performed, for example, by adding together between about 0.5 ng to about 1 ng RNA/miRNA and 0.001 nM to 0.01 nM of probe or probe mixture, incubating the reaction at 95° C. for 2 min (to dissociate duplexes), 65° C. for 2 min (to anneal), and then placing the reaction on ice. The amplification reaction is then performed, for example, by adding the annealed reaction with 2 μL of 10× buffer, 0.4 μL of 10 mg/mL BSA, 0.4 μL of 10 mM dNTPs, 1-2 μL phi29, and H₂O (to obtain a total reaction volume of 20 μL). The reaction is then incubated at room temperature (or up to about 30° C.) from between 1 hour to overnight. Another approach is to perform the annealing and the amplification concurrently (or in one step). The amplification can be performed with labeled nucleotides (e.g., fluorescently labeled nucleotides). The fluorescence can be measured using high tech laboratory instruments or hand-held fluorescent meters. Single probe specific for one miRNA or a mixture of multiple probes specific for multiple miRNAs can be used. The one step annealing and amplification reaction is performed by, for example, adding between about 0.5 ng to about 1 ng RNA/miRNA, 0.001 nM to 0.01 nM of probe or probe mixture, 2 μL of 10× buffer, 0.4 μL of 10 mg/mL BSA, 0.4 μL of 10 mM dNTPs, 1-2 μL phi29, and H₂O (to obtain a total reaction volume of 20 μL), followed by incubation at room temperature (or up to about 30° C.) from between 1 hour to overnight. An example of a circularized probe sequence used in this experiment is 5′ CCAGTGAATGCGAGTCCGACTAGGAGAGTAGGACAGCAGCCGT (SEQ ID NO: 81). Examples of miRNAs and probe sequences used in this experiment are: miR-22-3p and probe sequence (circularized) 5′ ACAGTTCTTCAACTGGCAGCTTCCAGTGAATGCGAGTCCGACTAGGAGAGTAGGACAGC AGCCGT (SEQ ID NO: 82); miR-451 and probe sequence (circularized) 5′ AACTCAGTAATGGTAACGGTTTCCAGTGAATGCGAGTCCGACTAGGAGAGTAGGACAG CAGCCGT (SEQ ID NO: 171); and miR-39 from C. elegans and probe sequence (circularized) 5′ CAAGCTGATTTACACCCGGTGACCAGTGAATGCGAGTCCGACTAGGAGAGTAGGACAG CAGCCGT (SEQ ID NO: 172). Examples of primers that can be used in this experiment are 5′ GAGAGTAGGACAGCAGCCG (SEQ ID NO: 173) for a forward primer and 5′ GGACTCGCATTCACTGG (SEQ ID NO: 174) for a reverse primer. The primers amplify the probes across the miRNA targets. Other primers that are specific for particular miRNAs can be designed by starting in the common probe (e.g., universal probe) and spanning through specific miRNA sequences. The primer design allows for quantitative PCR that is specific for a particular miRNA. This experiment can also be used to detect miRNAs and heart-specific proteins (e.g., BNP (B-type natriuretic peptide) or NT-pro-BNP (N-terminal pro b-type natriuretic peptide)), for example, by using rolling circle amplification (e.g., with miRNA specific probes and/or aptamer containing probes).

### Example 7: MiRNA Markers in Acetazolamide Treated Patients and Exercise Treated Patients

For this experiment, sample size determination was obtained from a G* Power software (FIG. 17). To determine sample size, the G power software used a 10 feature test that is two-tailed, has B power of 80%, a error probability of 0.10/10=0.01 (with correction of the numbers of features), a sensitivity (effect size) of 0.68 with 30 paired samples (obtained from the exercise treated group from Example 2), or a sensitivity of 0.86 with 20 paired samples (obtained from the oxygen treated group from Example 2). Based on the G* Power software, the sample size determined for this experiment was about 30 paired samples.

For this experiment, MiRNA markers ((miR-22-3p relative to miR-451a) or (miR-22-3p+miR-21-5p relative to Spike-RNA+miR-451a)) are tested on validation sample cohorts from acetazolamide-treated patients and on a cohort of 30 paired samples from an exercise intervention study. Paired data analysis is performed by comparing marker values before and after exercise training intervention. Paired data analysis is performed by comparing marker values for the placebo and the acetazolamide treatment groups. Changes in the markers for all data pairs are then determined. The data are separated based on the directional change (e.g., downward change or upward change). The groups (upregulated vs. downregulated) are then compared with the 6-minute walking distance at baseline. Body mass index and gender information are also taken into account. The fold change is analyzed and compared with the 6-minute walking distance at baseline. Paired data are analyzed with the Wilcoxon matched pairs signed rank test and the correlations are calculated with Spearman's rank correlation test (GraphPad Prism software). P<0.05 is considered significant.

### Example 8: Expansion of miRNA Marker Portfolio

MiRNAs such as hypoxamiRs (e.g., miR-135a, miR-186 (e.g., miR-186-5p), miR-210 (e.g., miR-210-3p), miR-199a-5p, miR-214 (e.g., miR-214-3p), or miR-204), muscle miRs (e.g., miR-378 (e.g., miR-378a-3p), miR-29a (e.g., miR-29a-3p), or miR-26a), erythrocyte miRs (e.g., miR-144), and/or other identifiable miRNAs are tested. MiRNA species are detectable in the plasma or serum samples of subjects by qPCR. Some miRNA levels change (up-regulation or downregulation) depending on the treatment intervention.

Correlation matrix analysis is performed by correlating fold-change values for each miRNA value (miRNA relative to Spike RNA) and/or with other miRNAs (R statistics platform). This correlation allows for the detection of miRNAs that are regulated in the same pathway or in parallel pathways because such miRNAs are significantly correlated. Identifying miRNAs that are separately regulated and likely work in unique pathways is also possible because these miRNAs are not correlated with other miRNAs. Based on this information, expansion of miRNA markers is feasible by either developing new marker sets, or by adding more miRNAs as potential markers. Correlation analysis is performed with the non-parametric Spearman's Rank Correlation test to avoid errors due to very high or very low valued outlier data.

For the correlation analysis, power calculation can be conducted using G-power (e.g., G* Power software). Because Spearman's rank correlation coefficient is computationally comparable to Pearson product-moment coefficient, power analysis can be conducted using software for estimating power of a Pearson's correlation. Similar power calculations can be conducted online with ANZMTG Statistical Decision Tree. For a two tailed test (an alpha of 0.05, a power of 0.80) and an effect size of r=0.5, the required sample size is 29, for an effect size of r=0.6, the sample size is 19, and for an effect size of r=0.7, the sample size is 13.

### Example 9: Identifying miRNAs as Potential PH Markers

RNA from 20 randomly selected pairs from: (a) oxygen or placebo treated patients; and (b) before and after exercise intervention study are evaluated with an Omics-tool to develop miRNA markers. An miRNA array that consists of up to 150 miRNA species or miRNA sequencing (RNAseq) can be considered for Omics-tool.

The primary goal of this assay is to identify differentially expressed miRNAs by clustering. Four different analyses are determined: a) fold-change in miRNA expression level vs. sample ID; b) miRNA expression levels standardized to a Spike-in synthetic RNA vs. intervention (e.g., before exercise, after exercise, treatment with placebo or with oxygen); c) fold-change in miRNA expression vs. 6-minute walking distance at baseline; and d) miRNA expression levels standardized to a Spike-in synthetic RNA vs. 6-minute walking distance at baseline. The miRNAs identified as potential candidates are then validated for incorporation into an miRNA marker portfolio (refer to Example 8).

Plate miRNA array: Following RT-qPCR, plate miRNA array data are standardized against Spike RNA. RNA sequencing: Isolated RNA from 0.5 mL of plasma/serum is Spiked with synthetic RNA (for quantification) and sent to a commercial provider (e.g., LC Sciences) to produce a library and perform sequencing. Generated reads are aligned and the number of reads are calculated by the provider. In addition, other available tools (e.g., TopHat, Bowtie aligners and cufflinks) are used to calculate reads per analyte. Quantification software tools such as Salmon & Kallisto are also considered. The reads per analyte for each sample are standardized by adjusting for the total number of reads, and for the reads measured for the Spiked-in synthetic RNA. Based on a heart failure study, groups of 10-27 serum/plasma samples are expected to be sufficient to reach statistical significance.

Data analysis is performed to calculate principal component analysis and hierarchical clustering with available online R-based tools such as ClustVis or ClusterEng. For the principal component analysis, unit variance scaling is applied to rows. Singular value decomposition with imputation is used to calculate principal components 1 (X-axis) and 2 (Y-axis). Unsupervised hierarchical clustering is performed using Euclidean distance. Identifying miRNA species that cluster together and that can demonstrate a significant change due to the intervention studies is also possible. In addition, identifying miRNAs that cluster based on the 6-minute walking distance at baseline can also be evaluated. The miRNAs identified in this example can then be tested as potential miRNA markers for pulmonary hypertension or as part of an miRNA marker portfolio (see Example 8).

### Embodiments

The following non-limiting embodiments provide illustrative examples of the disclosure, but do not limit the scope of the disclosure.

Embodiment 1. A method comprising: obtaining an expression level of miR-22-3p in a biological sample from a subject, and determining whether the biological sample indicates pulmonary hypertension based on the expression level.

Embodiment 2. The method of embodiment 1, further comprising performing an assay on the biological sample from the subject to obtain the expression level of miR-22-3p in the biological sample.

Embodiment 3. The method of embodiment 2, wherein the assay is sequencing.

Embodiment 4. The method of embodiment 2, wherein the assay is PCR.

Embodiment 5. The method of embodiment 2, wherein the assay is in situ hybridisation.

Embodiment 6. The method of embodiment 2, wherein the assay is microarray.

Embodiment 7. The method of any one of embodiments 1-6, further comprising obtaining an expression level of another miRNA in the biological sample, wherein the other miRNA is associated with pulmonary hypertension.

Embodiment 8. The method of any one of embodiments 1-7, wherein the pulmonary hypertension is pulmonary arterial hypertension.

Embodiment 9. The method of any one of embodiments 1-8, wherein the biological sample is a blood sample.

Embodiment 10. The method of any one of embodiments 1-9, wherein the biological sample is a serum sample.

Embodiment 11. The method of any one of embodiments 1-10, wherein the biological sample is a plasma sample.

Embodiment 12. A method comprising: a) extracting an miRNA associated with pulmonary hypertension from a biological sample from a subject with pulmonary hypertension; b) amplifying the miRNA; c) determining an expression level of the miRNA; and d) determining an miRNA clustering profile on the expression level of the miRNA.

Embodiment 13. The method of embodiment 12, wherein the miRNA is miR-22-3p.

Embodiment 14. The method of embodiment 12, wherein the miRNA is miR-21-5p.

Embodiment 15. The method of embodiment 12, wherein the miRNA is miR-451.

Embodiment 16. The method of any one of embodiments 12-15, wherein the biological sample is a blood sample.

Embodiment 17. The method of any one of embodiments 12-15, wherein the biological sample is a serum sample.

Embodiment 18. The method of any one of embodiments 12-15, wherein the biological sample is a plasma sample.

Embodiment 19. The method of any one of embodiments 12-18, wherein the pulmonary hypertension is pulmonary arterial hypertension.

Embodiment 20. A method comprising: a) obtaining an expression level of an miRNA in a first biological sample from a subject before the subject receives a treatment for pulmonary hypertension, wherein the subject has pulmonary hypertension; b) obtaining an expression level of the miRNA in a second biological sample from the subject after the subject receives the treatment for pulmonary hypertension; and c) observing a difference in the expression level of the miRNA in the first biological sample and the expression level of the miRNA in the second biological sample.

Embodiment 21. The method of embodiment 20, wherein the miRNA is associated with pulmonary hypertension.

Embodiment 22. The method of embodiment 20, wherein the miRNA is miR-22-3p.

Embodiment 23. The method of embodiment 20, wherein the miRNA is miR-21-5p.

Embodiment 24. The method of embodiment 20, wherein the miRNA is miR-451.

Embodiment 25. The method of any one of embodiments 20-24, further comprising performing a first assay on the first biological sample from the subject to obtain the expression level of the miRNA in the first biological sample, and performing a second assay on the second biological sample from the subject to obtain the expression level of the miRNA in the second biological sample.

Embodiment 26. The method of embodiment 25, wherein the first assay and the second assay are sequencing.

Embodiment 27. The method of embodiment 25, wherein the first assay and the second assay are PCR.

Embodiment 28. The method of embodiment 25, wherein the first assay and the second assay are in situ hybridisation.

Embodiment 29. The method of embodiment 25, wherein the first assay and the second assay are microarray.

Embodiment 30. The method of any one of embodiments 20-29, wherein the pulmonary hypertension is pulmonary arterial hypertension.

Embodiment 31. The method of any one of embodiments 20-29, wherein the first biological sample and the second biological sample are blood samples.

Embodiment 32. The method of any one of embodiments 20-29, wherein the first biological sample and the second biological sample are serum samples.

Embodiment 33. The method of any one of embodiments 20-29, wherein the first biological sample and the second biological sample are plasma samples.

Embodiment 34. The method of any one of embodiments 20-33, wherein the treatment for pulmonary hypertension is exercise training.

Embodiment 35. The method of any one of embodiments 20-33, wherein the treatment for pulmonary hypertension is a therapeutic drug.

Embodiment 36. The method of any one of embodiments 20-33, wherein the treatment for pulmonary hypertension is oxygen therapy with gas that is at least 21% oxygen.

Embodiment 37. A method comprising: a) assaying a biological sample of a subject; b) quantifying an expression level of an miRNA in the biological sample, wherein the miRNA is associated with pulmonary hypertension; and c) determining a risk score of the subject for pulmonary hypertension based at least partially on the expression level.

Embodiment 38. The method of embodiment 37, wherein the miRNA is miR-22-3p.

Embodiment 39. The method of embodiment 37, wherein the miRNA is miR-21-5p.

Embodiment 40. The method of embodiment 37, wherein the miRNA is miR-451.

Embodiment 41. The method of any one of embodiments 37-40, wherein the quantifying comprises sequencing.

Embodiment 42. The method of any one of embodiments 37-40, wherein the quantifying comprises PCR.

Embodiment 43. The method of any one of embodiments 37-40, wherein the quantifying comprises in situ hybridisation.

Embodiment 44. The method of any one of embodiments 37-40, wherein the quantifying comprises microarray.

Embodiment 45. The method of any one of embodiments 37-44, wherein the pulmonary hypertension is pulmonary arterial hypertension.

Embodiment 46. The method of any one of embodiments 37-45, wherein the biological sample is a serum sample.

Embodiment 47. The method of any one of embodiments 37-45, wherein the biological sample is a plasma sample.

Embodiment 48. A kit comprising: a) an oligonucleotide that binds an miRNA associated with pulmonary hypertension; b) a reagent for amplifying the miRNA; and c) written instructions for quantifying an expression level of the miRNA and for identifying pulmonary hypertension based on the expression level.

Embodiment 49. The kit of embodiment 48, wherein the miRNA associated with pulmonary hypertension is miR-22-3p.

Embodiment 50. The kit of embodiment 48, wherein the miRNA associated with pulmonary hypertension is miR-21-5p.

Embodiment 51. The kit of embodiment 48, wherein the miRNA associated with pulmonary hypertension is miR-451.

Embodiment 52. The kit of any one of embodiments 48-51, wherein the reagent is a DNA polymerase.

Embodiment 53. A method comprising: a) annealing a circularized probe to an miRNA associated with pulmonary hypertension, wherein the circularized probe comprises a sequence that is at least partially complementary to the miRNA, thereby generating an annealed complex; and b) incubating the annealed complex with a polymerase to generate an amplified rolling circle amplification product.

Embodiment 54. The method of embodiment 53, wherein the annealing and the incubating are performed concurrently.

Embodiment 55. The method of any one of embodiments 53-54, further comprising sequencing the amplified rolling circle amplification product.

Embodiment 56. The method of any one of embodiments 53-55, wherein the incubating is performed at room temperature.

Embodiment 57. The method of any one of embodiments 53-56, wherein the incubating is performed for about 2 hours.

Embodiment 58. The method of any one of embodiments 53-57, wherein the circularized probe comprises 2 nucleotide modifications compared to a universal circularized probe that targets the miRNA associated with pulmonary hypertension.

Embodiment 59. The method of any one of embodiments 53-58, wherein the miRNA associated with pulmonary hypertension is miR-22-3p.

Embodiment 60. The method of any one of embodiments 53-59, wherein the miRNA associated with pulmonary hypertension is miR-21-5p.

Embodiment 61. The method of any one of embodiments 53-60, wherein the miRNA associated with pulmonary hypertension is miR-451.

Embodiment 62. The method of any one of embodiments 53-61, wherein the polymerase is phi29.

Embodiment 63. The method any one of embodiments 53-62, wherein the pulmonary hypertension is pulmonary arterial hypertension.

In some embodiments, the disclosure provides a method comprising: obtaining an expression level of miR-22-3p in a biological sample from a subject and determining a status of pulmonary hypertension (PH) of the subject based at least partially on the expression level. In some embodiments, the method further comprises comparing the expression level of miR-22-3p to a reference. In some embodiments, the reference is the expression level of miR-22-3p in a biological sample from a healthy subject or a subject with pulmonary hypertension. In some embodiments, the reference is the expression level of miR-22-3p in a biological sample taken from the subject at a different time point. In some embodiments, the subject is determined to have PH when the expression level of miR-22-3p is reduced or increased as compared to the reference. In some embodiments, the obtaining comprises an expression array, next generation sequencing (NGS), quantitative reverse transcriptase PCR, northern blot analysis, real-time PCR, in situ hybridization, RNase protection, microarray, or a combination thereof. In some embodiments, the method further comprises obtaining an expression level of at least one more miRNA associated with pulmonary hypertension in the biological sample. In some embodiments, the method further comprises comparing a ratio between the expression level of miR-22-3p to the expression level of the at least one more miRNA associated with pulmonary hypertension. In some embodiments, the method further comprises comparing the ratio to a reference. In some embodiments, the reference is the ratio between the expression level of miR-22-3p to the expression level of the at least one more miRNA associated with pulmonary hypertension in a biological sample from a healthy subject or a subject with pulmonary hypertension. In some embodiments, the reference is the ratio between the expression level of miR-22-3p to the expression level of the at least one more miRNA associated with pulmonary hypertension in a biological sample from the subject at a different time point. In some embodiments, the subject is determined to have PH when the ratio is reduced or increased as compared to the reference.

In some embodiments, the disclosure provides a method comprising: obtaining a first expression level of an miRNA in a first biological sample from a subject with pulmonary hypertension; obtaining a second expression level of the miRNA in a second biological sample from the subject after the subject receives a treatment for pulmonary hypertension; and observing a change in expression levels of the miRNA based at least partially on the first expression level and the second expression level. In some embodiments, (a) is performed before (b). In some embodiments, (a) is performed after commencement of the treatment but before (b). In some embodiments, (a) is performed before commencement of the treatment. In some embodiments, the miRNA is an miRNA associated with pulmonary hypertension. In some embodiments, the miRNA associated with pulmonary hypertension is miR-22-3p. In some embodiments, the miRNA associated with pulmonary hypertension is miR-21-5p. In some embodiments, the miRNA associated with pulmonary hypertension is miR-451. In some embodiments, the treatment for pulmonary hypertension comprises exercise training, at least 21% oxygen therapy, a therapeutic drug, or any combination thereof. In some embodiments, the first biological sample and the second biological sample are plasma samples. In some embodiments, the first biological sample and the second biological sample are serum samples.

In some embodiments, the disclosure provides a method comprising: a) assaying a biological sample from a subject; b) quantifying an expression level of an miRNA in the biological sample, wherein the miRNA is associated with pulmonary hypertension; and c) determining a risk score of the subject for pulmonary hypertension based at least partially on the expression level. In some embodiments, the miRNA associated with pulmonary hypertension is miR-22-3p. In some embodiments, the miRNA associated with pulmonary hypertension is miR-21-5p. In some embodiments, the miRNA associated with pulmonary hypertension is miR-451. In some embodiments, the quantifying comprises an expression array, next generation sequencing (NGS), quantitative reverse transcriptase PCR, northern blot analysis, real-time PCR, in situ hybridization, RNase protection, microarray, or a combination thereof. In some embodiments, the biological sample is a serum sample. In some embodiments, the biological sample is a plasma sample. In some embodiments, the method further comprises comparing the expression level of the miRNA associated with pulmonary hypertension to a reference. In some embodiments, the reference is the expression level of the miRNA associated with pulmonary hypertension in a biological sample from a healthy subject or a subject with pulmonary hypertension. In some embodiments, the reference is the expression level of the miRNA associated with pulmonary hypertension in a biological sample taken from the subject at a different time point. In some embodiments, the risk score is determined based at least on the comparing.

In some embodiments, the disclosure provides a kit comprising: (a) an oligonucleotide that binds an miRNA associated with pulmonary hypertension; (b) a reagent for amplifying the miRNA; and (c) written instructions for quantifying an expression level of the miRNA and for identifying pulmonary hypertension based at least partially on the expression level. In some embodiments, the miRNA associated with pulmonary hypertension is miR-22-3p. In some embodiments, the miRNA associated with pulmonary hypertension is miR-21-5p. In some embodiments, the miRNA associated with pulmonary hypertension is miR-451. In some embodiments, the reagent comprises a DNA polymerase.

In some embodiments, the disclosure provides a method comprising: (a) extracting a plurality of miRNAs from a biological sample from a subject with pulmonary hypertension; (b) amplifying at least one miRNA in the plurality of miRNAs; (c) determining an expression level of the at least one miRNA; (d) determining an miRNA clustering profile based at least partially on the expression level of the miRNA in comparison to a reference; and (e) identifying the miRNA as indicative of a status of pulmonary hypertension in the subject. In some embodiments, the reference is a database. In some embodiments, the reference comprises the expression level of the miRNA that was previously taken from the subject, from a healthy subject, from a subject with pulmonary hypertension, from a population of healthy subjects, from a population of subjects with pulmonary hypertension, or any combination thereof. In some embodiments, the at least one miRNA comprises at least two, at least three, at least four, at least five, at least six, at least seven, at least eight, at least nine, at least ten, or more miRNAs. In some embodiments, the status comprises early-onset PH, late-onset PH, absence of PH determination, or presence of PH. In some embodiments, the at least one miRNA is an miRNA associated with pulmonary hypertension. In some embodiments, the miRNA associated with pulmonary hypertension is miR-22-3p. In some embodiments, the miRNA associated with pulmonary hypertension is miR-21-5p. In some embodiments, the miRNA associated with pulmonary hypertension is miR-451. In some embodiments, the biological sample is a serum sample. In some embodiments, the biological sample is a plasma sample.

In some embodiments, the disclosure provides a method comprising: a) annealing a circularized probe to an miRNA associated with pulmonary hypertension, wherein the circularized probe comprises a sequence that is at least partially complementary to the miRNA, thereby generating an annealed complex; and b) incubating the annealed complex with a polymerase to generate an amplified rolling circle amplification product. In some embodiments, the annealing and the incubating is performed concurrently. In some embodiments, the method further comprises sequencing the amplified rolling circle amplification product. In some embodiments, the incubating is performed at room temperature. In some embodiments, the incubating is performed for about 2 hours. In some embodiments, the circularized probe comprises 2 nucleotide modifications compared to an universal circularized probe that targets the miRNA associated with pulmonary hypertension. In some embodiments, the miRNA associated with pulmonary hypertension is miR-22-3p. In some embodiments, the miRNA associated with pulmonary hypertension is miR-21-5p. In some embodiments, the miRNA associated with pulmonary hypertension is miR-451. In some embodiments, the polymerase is phi29. In some embodiments, the pulmonary hypertension is pulmonary arterial hypertension.

1-63. (canceled)

64. A method comprising: obtaining an expression level of miR-22-3p in a biological sample from a subject, and determining whether the biological sample indicates pulmonary hypertension based on the expression level.

65. The method of claim 64, further comprising performing an assay on the biological sample from the subject to obtain the expression level of miR-22-3p in the biological sample.

66. The method of claim 65, wherein the assay is sequencing.

67. The method of claim 65, wherein the assay is PCR.

68. The method of claim 65, wherein the assay is in situ hybridization.

69. The method of claim 65, wherein the assay is microarray.

70. The method of claim 64, wherein the pulmonary hypertension is pulmonary arterial hypertension.

71. The method of claim 64, wherein the biological sample is a blood sample.

72. The method of claim 64, wherein the biological sample is a serum sample.

73. The method of claim 64, wherein the biological sample is a plasma sample.

74. The method of claim 64, further comprising obtaining an expression level of another miRNA in the biological sample, wherein the other miRNA is associated with pulmonary hypertension.

75. The method of claim 74, wherein the another miRNA is miR-21-5p.

76. The method of claim 74, wherein the another miRNA is miR-451.

77. The method of claim 64, further comprising providing a treatment to the subject determined to have pulmonary hypertension.

78. The method of claim 77, wherein the treatment for pulmonary hypertension is exercise training.

79. The method of claim 77, wherein the treatment for pulmonary hypertension is a therapeutic drug.

80. The method of claim 77, wherein the treatment for pulmonary hypertension is oxygen therapy with gas that is at least 21% oxygen.

81. The method of claim 77, wherein the treatment for pulmonary hypertension comprises use of a breathing stabilizer.

82. The method of claim 64, further comprising classifying the pulmonary hypertension of the subject based at least partially on the expression level.

83. The method of claim 82, wherein prior to the classifying the pulmonary hypertension, the expression level is compared to a reference.
